(19) United States
(12) Patent Application Publication
(10) Pub. No.: US 2006/0122138 A1
(43) Pub. Date: Jun. 8, 2006

(54) Modulators of EphA2 and EphrinA1 for the treatment of fibrosis-related disease

(75) Inventors: Michael S. Kinch, Laytonsville, MD (US); Kelly Carles-Kinch, Laytonsville, MD (US)

Correspondence Address: JOHNATHAN KLEIN-EVANS, ONE MEDIMMUNE WAY, GAITHERSBURG, MD, 20878, US

(73) Assignee: MEDIMMUNE, INC., Gaithersburg, MD (US)

(21) Appl. No.: 11/259,267

(22) Filed: Oct. 27, 2005

Related U.S. Application Data
(60) Provisional application No. 60/622,517, filed on Oct. 27, 2004.

(51) Int. Cl. A61K 48/00; A61K 39/395; A61K 39/00

(52) U.S. Cl. 514/44; 424/155.1; 424/185.1

(57) ABSTRACT

The present invention relates to methods and compositions designed for the treatment, management, prevention and/or amelioration of non-neoplastic hyperproliferative epithelial and/or endothelial cell disorders, including but not limited to disorders associated with increased deposition of extracellular matrix components (e.g., collagen, proteoglycans, tenascin and fibronectin) and/or aberrant angiogenesis. Non-limiting examples of such disorders include cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis. The methods of the invention comprise the administration of an effective amount of one or more agents that are modulators of EphA2 and/or its endogenous ligand, EphrinA1. The invention also provides pharmaceutical compositions comprising one or more EphA2/EphrinA1 Modulators of the invention either alone or in combination with one or more other agents useful for therapy for such non-neoplastic hyperproliferative epithelial and/or endothelial disorders. Diagnostic methods and methods for screening for EphA2/EphrinA1 Modulators are also provided.

This application claims priority to U.S. Provisional Patent Application Ser. No. 60/622,517, filed Oct. 27, 2004, which is incorporated by reference herein in its entirety.

### 1. FIELD OF THE INVENTION

The present invention relates to methods and compositions designed for the treatment, management, or prevention of non-neoplastic hyperproliferative epithelial and/or endothelial cell disorders, including but not limited to, disorders associated with increased deposition of extracellular matrix components (e.g., collagen, proteoglycans, tenascin and fibronectin) and/or aberrant (i.e., increased) angiogenesis. Nor-limiting examples of such disorders include cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis. The invention provides methods of preventing, treating or managing a non-neoplastic hyperproliferative epithelial and/or endothelial cell disorder, the methods comprising the administration of an effective amount of an agent that modulates the expression and/or activity(ies) of EphA2 and/or its endogenous ligand, EphrinA1. In accordance with the invention, one or more other therapies can be administered in combination with an agent that modulates the expression and/or activity(ies) of EphA2 and/or its endogenous ligand, EphrinA1, to treat, prevent or manage a non-neoplastic hyperproliferative epithelial and/or endothelial cell disorder.

The invention also provides pharmaceutical compositions comprising an agent that modulates the expression and/or activity of EphA2 and/or its endogenous ligand, EphrinA1. The pharmaceutical compositions of the invention can further comprise one or more other prophylactic or therapeutic agents for the prevention, treatment and/or management of a non-neoplastic hyperproliferative epithelial and/or endothelial cell disorder. Such pharmaceutical compositions are useful in the prevention, treatment and/or management of a non-neoplastic hyperproliferative epithelial and/or endothelial cell disorder. The invention further provides diagnostic methods and methods for screening for prophylactically and/or therapeutically useful agents.

### 2. BACKGROUND OF THE INVENTION

EphA2

EphA2 (epithelial cell kinase) is a 130 kDa member of the Eph family of receptor tyrosine kinases (Zantek N. et al, 1999, Cell Growth Differ. 10:629-38; Lindberg R. et al., 1990, Mol. Cell. Biol. 10:6316-24). The function of EphA2 is not known, but it has been suggested to regulate proliferation, differentiation, and barrier function of colonic epithelium (Rosenberg et al., 1997, Am. J. Physiol. 273:G824-32), vascular network assembly, endothelial migration, capillary morphogenesis, and angiogenesis (Stein et al., 1998, Genes Dev. 12:667-78), nervous system segmentation and axon pathfinding (Bovenkamp D. and Greer P., 2001, DNA Cell Biol. 20:203-13), tumor neovascularization (Ogawa K. et al., 2000, Oncogene 19:6043-52), and cancer metastasis (International Patent Publication Nos. WO 01/9411020, WO 96/36713, WO 01/12840, WO 01/12172).

The natural ligand of EphA2 is EphrinA1 (Eph Nomenclature Committee, 1997, Cell 90(3):403-4; Gale, et al., 1997, Cell Tissue Res. 290(2): 227-41). The EphA2 and EphrinA1 interaction is thought to help anchor cells on the surface of an organ and also down regulate epithelial and/or endothelial cell proliferation by decreasing EphA2 expression through EphA2 autophosphorylation (Lindberg et al., 1990, supra). Under natural conditions, the interaction helps maintain an epithelial cell barrier that protects the organ and helps regulate over proliferation and growth of epithelial cells. However, there are disease states that prevent epithelial cells from forming a protective barrier or cause the destruction and/or shedding of epithelial and/or endothelial cells and thus prevent proper healing from occurring.

Fibrosis

Progressive fibrosis of liver, kidney, lungs, and other viscera often results in organ failure leading to death or the need for transplantation. These diseases affect millions in the United States and worldwide. For example, hepatic fibrosis is the leading non-malignant gastrointestinal cause of death in the United States. Moreover, it has been increasingly recognized that progression of fibrosis is the single most important determinant of morbidity and mortality in patients with chronic liver disease (Poynard, T. P. et al., 1997, Lancet 349:825-832). Fibrosis is characterized by excessive deposition of matrix components. This leads to destruction of normal tissue architecture and compromised tissue function.

Pulmonary fibrosis can be caused by damaging agents and is associated with hypersensitivity pneumonitis and a strong inflammatory response. Idiopathic pulmonary fibrosis (IPF) is associated with desquamative interstitial pneumonitis (DIP), characterized by mononuclear cells in the alveoli and little cellular infiltrate in the interstitium. IPF is also associated with usual interstitial pneumonitis (UIP), characterized by patchy interstitial infiltrate and thickening of alveolar walls. The histology of pulmonary fibrosis includes alveolar wall thickening (which may include a “honeycombing” effect), metaplastic epithelium, and changes to fibroblasts including proliferation/ECM accumulation, myofibroblast differentiation, and fibroblastic foci.

Wound healing and fibrosis follow similar pathways. Both involve damage to the epithelium, followed by proliferation and differentiation of fibroblasts and ECM deposition. Both are mediated by cell signaling messengers such as TGFβ and PDGF. In wound healing, tissue regeneration ceases once the wound is healed; however, in fibrosis, cell growth does not stop, leading to continued ECM deposition and a lack of protease activity. Bleomycin induces lung epithelial cell death, followed by acute neutrophilic influx, subsequent chronic inflammation, and parenchymal fibrosis within 4 weeks of administration to susceptible strains of mice. Bleomycin-treated lung epithelial cells as a model for lung fibrosis replicates key pathologic features of human IPF, including fibroproliferation within the lung parenchyma and other pathologic conditions (Dunsmore and Shapiro, 2004, J. Clin. Invest. 113:180-182). Fibrosis induced by bleomycin can be prevented by addition of soluble Fas, which blocks Fas-mediated apoptosis (Kuwano, et al., 1999, J. Clin. Invest. 104:13-9). Fas-mediated apoptosis in the epithelium of IPF tissue is characterized by an increase in Fas and/or Fas ligand. Correspondingly, factors such as soluble Fas that cause a decrease in epithelial apoptosis also show protection against fibrosis.

Asbestosis (interstitial fibrosis) is defined as diffuse lung fibrosis due to the inhalation of asbestos fibers. C. A. Staples, 1992, Radiologic Clinics of North America, 30(6):1195. It is one of the major causes of occupationally related lung damage. Merck Index, 1999 (17^{th} ed.), 622. Asbestosis characteristically occurs following a latent period of 15-20 years, with a progression of disease even after exposure has ceased, but rarely occurs in the absence of pleural plaques. C. Peacock, 2000, Clinical Radiology, 55: 425. Fibrosis first arises in and around the respiratory bronchioles, predominating in the subpleural portions of the lung in the lower lobes, and then progresses centrally. C. A. Staples, Radiologic Clinics of North America, 30 (6):1195, 1992. Asbestosis may cause an insidious onset of progressive dyspnea in addition to a dry cough. The incidence of lung cancer is increased in smokers with asbestosis, and a dose-response relationship has been observed. Merck Index, 1999 (17^{th} ed.), 623.

Additional therapeutics are needed to diagnose and treat fibrotic diseases. For example, no treatments for fibrotic lung diseases such as asbestosis are known to be effective.

Angiogenesis and Fibrosis

Angiogenesis is the formation of new blood vessels from preexisting vasculature, and is a multi-step process involving a diverse array of molecular signals. Ligands for receptor tyrosine kinases (RTKs), including the EphA RTKs, have been implicated as critical mediators of angiogenesis (Cheng et al., 2002, Mol. Cancer Res. 1:2-11). EphA2 interaction with its endogenous ligand, EphrinA1, has been shown to be necessary for maximal induction of vascular endothelial growth factor (VEGF)-mediated endothelial cell migration, survival, sprouting and neovascularization, (Cheng et al., 2002, Mol. Cancer Res. 1:2-11).

Recent observations suggest a possible link between angiogenesis and the pathology of fibrosis, particularly pulmonary fibrosis (Noble, W., 2003, Amer. J. Respiratory Cell & Mol. Biol. 29:S27-S31). Studies have demonstrated a correlation between increased angiogenic activity in the lung tissue of patients with idiopathic pulmonary fibrosis (IPF) and in experimental fibrosis (Keane et al., 1997, J. Immunol. 159:1437-1443). This increased angiogenic activity is believed to be attributed to an imbalance of certain pro-angiogenic chemokines (e.g., interleukin-8 (IL-8)) and anti-angiogenic chemokines (e.g., inducible protein-10 (IP-10)). IP-10 has been shown to be induced by IFN-γ, which in human and animal studies inhibits progressive pulmonary fibrosis.

Currently, conventional therapy for IPF most commonly consists of corticosteroids alone, an approach that has been suggested to lack efficacy and have a high degree of adverse side effects (Wurfel and Raghu, http://www.chestnet.org/education/onine/pccu/vol16/lessons13_{—}14lesson13.php). Thus, novel therapies are needed to treat fibrosis and diseases associated with aberrant angiogenesis.

### 3. SUMMARY OF THE INVENTION

The present invention is based, in part, on the discovery that agents that disrupt or decrease EphA2 binding to its endogenous ligand can prevent, reduce or slow the progression of non-neoplastic hyperproliferative epithelial cell and/or endothelial cell disorders, including, but not limited to, disorders associated with increased deposition of extracellular matrix (ECM) components and disorders associated with aberrant (i.e., increased or decreased) angiogenesis. Non-limiting examples of such disorders include cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis. Without being bound to a particular theory or mechanism, the disruption or decrease in EphA2 binding to its endogenous ligand (e.g., EphrinA1) may increase the proliferation, growth, and/or survival of EphA2-expressing epithelial cells, decrease the deposition of ECM components and/or maintain the organization of the epithelial cell layers by disrupting or decreasing ligand-induced EphA2 signaling and thus increasing EphA2 protein accumulation and/or stability. Alternatively, or in addition, without being bound to a particular theory or mechanism, the disruption or decrease in EphA2 binding to its endogenous ligand may inhibit or decrease angiogenesis, in particular vascular endothelial growth factor (VEGF)-induced angiogenesis.

The present invention provides methods for the prevention, management, treatment and/or amelioration of a non-neoplastic hyperproliferative epithelial cell and/or endothelial cell disorder (including, but not limited to, a disorder associated with increased deposition of extracellular matrix (ECM) components and a disorder associated with aberrant angiogenesis) or a symptom thereof, the methods comprising administering to a subject in need thereof an effective amount of an EphA2/EphrinA1 Modulator. The present invention also provides methods for the prevention, management, treatment and/or amelioration of a non-neoplastic hyperproliferative epithelial cell and/or endothelial cell disorder (including, but not limited to, a disorder associated with increased deposition of extracellular matrix (ECM) components and a disorder associated with aberrant angiogenesis) or a symptom thereof, the methods comprising administering to a subject in need thereof an effective amount of an EphA2/EphrinA1 Modulator and an effective amount of a therapy other than an EphA2/EphrinA1 Modulator (e.g., an analgesic agent, an anesthetic agent, an antibiotic, or an immunomodulatory agent).

Non-limiting examples of non-neoplastic hyperproliferative epithelial and/or endothelial cell disorders include cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis.

The invention provides modulators of EphA2 and/or EphrinA1 (“EphA2/EphrinA1 Modulators”). Non-limiting examples of EphA2/EphrinA1 Modulators are agents that confer a biological effect by modulating (directly or indirectly): (i) the expression of EphA2 and/or an endogenous ligand(s) of EphA2 (preferably, EphrinA1), at, e.g., the transcriptional, post-transcriptional, translational or post-translation level; and/or (ii) an activity(ies) of EphrinA1.

Examples of EphA2/EphrinA1 Modulators include, but are not limited to, agents that inhibit or reduce the interaction between EphA2 and an endogenous ligand(s) of EphA2, preferably, EphrinA1 (hereinafter “EphA2/EphrinA1 Interaction Inhibitors”). Non-limiting examples of EphA2/EphrinA1 Interaction Inhibitors include: (i) agents that bind to EphA2, prevent or reduce the interaction between the EphA2 and EphrinA1, and induce EphA2 signal transduction (e.g., soluble forms of EphrinA1 (e.g., in monomeric or multimeric form), antibodies that bind EphA2, induce signaling and phosphorylation of EphA2 (i.e., an EphA2 agonistic antibody)); (ii) agents that bind to EphA2, prevent or reduce the interaction between the EphA2 and EphrinA1, and prevent or induce very low to negligible levels of EphA2 signal transduction (e.g., EphA2 antagonistic antibodies and dominant negative forms of EphrinA1); (iii) agents that bind to EphrinA1, prevent or reduce the interaction between an EphA2 and EphrinA1, and induce EphrinA1 signal transduction (e.g., soluble forms of EphA2 and antibodies that bind to EphrinA1 and induce EphrinA1 signal transduction); and (iv) agents that bind to EphrinA1, prevent or reduce the interaction between an EphA2 and EphrinA1, and prevent or induce very low to negligible levels of EphrinA1 signal transduction (e.g., dominant negative forms of EphA2 and anti-EphrinA1 antibodies).

In further embodiments, EphA2/EphrinA1 Modulators include, but are not limited to, agents that modulate the expression of EphA2. Such agents can decrease/downregulate EphA2 expression (e.g., EphA2 antisense molecules, RNAi and ribozymes) or increase/upregulate EphA2 expression such that the amount of EphA2 on the cell surface exceeds the amount of endogenous ligand (preferably, EphrinA1) available for binding, and thus, increases the amount of unbound EphA2 (e.g., nucleic acids encoding EphA2)).

In other embodiments, EphA2/EphrinA1 Modulators are agents that modulate the expression of EphrinA1. Such agents can decrease/downregulate EphrinA1 expression (e.g., EphrinA1 antisense molecules, RNAi and ribozymes) or increase/upregulate EphrinA1 expression (e.g., nucleic acids encoding EphrinA1)).

In yet other embodiments, EphA2/EphrinA1 Modulators of the invention include, but are not limited to, agents that modulate the protein stability or protein accumulation of EphA2 or EphrinA1. In a preferred embodiment, an EphA2 or EphrinA1 Modulator of the invention increases protein stability and/or accumulation of EphA2.

In further embodiments, EphA2/EphrinA1 Modulators of the invention are agents that modulate kinase activity (e.g., of EphA2, EphrinA1 or of a heterologous protein known to associate with EphA2 or EphrinA1 at the cell membrane).

In further embodiments, EphA2/EphrinA1 Modulators of the invention include, but are not limited to, agents that bind to EphA2 and prevent or reduce EphA2 signal transduction but do not inhibit or reduce the interaction between EphA2 and EphrinA1 (e.g., an EphA2 intrabody); and agents that bind to EphrinA1 and prevent or reduce EphrinA1 signal transduction but do not inhibit or reduce the interaction between EphrinA1 and Eph EphA2 (e.g., an EphrinA1 antibody). In a preferred embodiment, EphA2/EphrinA1 Modulators of the invention decrease EphA2 cytoplasmic tail phosphorylation.

In a preferred embodiment of the invention, EphA2/EphrinA1 Modulators increase survival and/or growth of EphA2-expressing cells.

In another preferred embodiment of the invention, EphA2/EphrinA1 Modulators of the invention include, but are not limited to, dominant negative forms of EphA2; soluble forms of EphA2 (e.g., EphA2-Fc); Ephrin A1 antisense molecules; anti-EphA2 antibodies that bind to EphA2, interfere with EphA2-ligand interaction, and do not induce EphA2 signal transduction; and anti-EphrinA1 antibodies. In other embodiments, the anti-EphA2 and/or anti-EphrinA1 antibodies can be linked to a cytotoxic agent.

In a specific embodiment, an EphA1/EphrinA1 Modulator is not an agent that decreases the expression of EphA2. In another embodiment, an EphA2/EphrinA1 Modulator is not an agent that modulates the protein stability or protein accumulation of EphA2. In another embodiment of the invention, an EphA2/EphrinA1 Modulator is not an agent that modulates kinase activity (e.g., of EphA2, EphrinA1 or of a heterologous protein known to associate with EphA2 or EphrinA1 at the cell membrane). In another embodiment, an EphA2/EphrinA1 Modulator is not an EphA2 agonistic antibody. In a further embodiment, an EphA2/EphrinA1 Modulator is not an EphA2 antisense molecule. In yet a further embodiment, an EphA2/EphrinA1 Modulator is not a soluble form of EphrinA1 or a fragment thereof.

In another specific embodiment, the invention provides methods for preventing, treating or managing cirrhosis or fibrosis (e.g., fibrosis of the liver, kidney, lungs, heart, retina and other viscera) in a subject in need thereof, said methods comprising administering to a subject an effective amount of one or more EphA2/EphrinA1 Modulators of the invention. In a further specific embodiment, the invention provides methods for preventing, treating or managing cirrhosis or fibrosis (e.g., fibrosis of the liver, kidney, lungs, heart, retina and other viscera) in a subject in need thereof, said methods comprising administering to a subject an effective amount of an EphA2/EphrinA1 Modulator and an effective amount of a therapy other than an EphA2/EphrinA1 Modulator.

In a specific embodiment, the invention provides a method of preventing, managing, treating or ameliorating asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis in a subject in need thereof comprising administering an effective amount of an EphA2/EphrinA1 Modulator. In another embodiment, the invention provides a method of preventing, managing, treating or ameliorating asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis comprising administering an effective amount of an EphA2/EphrinA1 Modulator and an effective amount of a therapy other than an EphA2/EphrinA1 Modulator.

The present invention provides methods for the screening and identification of EphA2/EphrinA1 Modulators that modulate (e.g., increase or decrease the expression and/or activity) EphA2 and/or EphrinA1, e.g., decrease EphA2-endogenous ligand binding, decrease EphrinA1 gene expression, upregulate EphA2 gene expression, increase EphA2 protein stability or protein accumulation, decrease EphA2 cytoplasmic tail phosphorylation, increase proliferation of EphA2 expressing cells, increase survival of EphA2-expressing cells (e.g., by preventing apoptosis), maintain/reconstitute the integrity of an epithelial and/or endothelial cell layer, and/or prevent or slow angiogenesis. In a specific embodiment, the invention provides methods for screening and identifying EphA2/EphrinA1 Modulators that prevent and/or slow the progression of non-neoplastic hyperproliferative epithelial and/or endothelial cell disorders such as cirrhosis, fibrosis (e.g., fibrosis of the liver, kidney, lungs, heart, retina and other viscera) by preventing or slowing the deposition of ECM components (e.g., collagen) in the epithelial and/or endothelial cell layers. In another embodiment, the invention provides methods for screening and identifying EphA2/EphrinA1 Modulators that prevent and/or slow the progression of non-neoplastic hyperproliferative epithelial and/or endothelial cell disorders, such as cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis by modulating angiogenesis.

In a specific embodiment, the invention provides methods of preventing, reducing or slowing down angiogenesis in a subject in need thereof comprising the administration of one or more EphA2/EphrinA1 Modulators of the invention alone or in combination with one or more other prophylactic or therapeutic agents that are not EphA2/EphrinA1 Modulator-based. In an alternative embodiment, the invention provides methods of increasing or upregulating angiogenesis in a subject in need thereof comprising the administration of one or more EphA2/EphrinA1 Modulators of the invention alone or in combination with one or more other prophylactic or therapeutic agents that are not EphA2/EphrinA1 Modulator-based.

The present invention provides pharmaceutical compositions and prophylactic and therapeutic regimens designed to treat, manage, or prevent non-neoplastic hyperproliferative epithelial and/or endothelial cell disorders such as cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis. In a specific embodiment, the present invention provides pharmaceutical compositions and prophylactic and therapeutic regimens that prevent or slow down the deposition of ECM components (e.g., collagen) in the epithelial and/or endothelial cell layers, and/or modulate angiogenesis, and the use of such compositions and regimens in the treatment, management or prevention of non-neoplastic hyperproliferative epithelial cell disorders, in particular fibrosis and/or fibrosis-related diseases. In a specific embodiment, the invention provides methods of preventing, reducing, or slowing down angiogenesis. In another specific embodiment, the invention provides methods of increasing or upregulating angiogenesis.

The invention further provides diagnostic methods using the EphA2/EphrinA1 Modulators of the invention to evaluate the efficacy of a therapy for a non-neoplastic hyperproliferative epithelial and/or endothelial cell disorder (e.g., cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis), wherein the therapy monitored can be either EphA2/EphrinA1 Modulator-based or not EphA2/EphrinA1 Modulator-based. In particular embodiments, the diagnostic methods of the invention provide methods of imaging areas of hyperproliferation. The diagnostic methods of the invention may also be used to prognose or predict non-neoplastic hyperproliferative epithelial and/or endothelial cell disorders (e.g., cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis). The EphA2/EphrinA1 Modulators of the invention may also be used for immunohistochemical analyses of frozen or fixed cells or tissue assays.

The invention also provides kits comprising the pharmaceutical compositions or diagnostic reagents of the invention.

3.1 Terminology

As used herein, the term “agent” refers to a molecule that has a desired biological effect. Agents include, but are not limited to, proteinaceous molecules, including, but not limited to, peptides, polypeptides, proteins, post-translationally modified proteins, antibodies etc.; small molecules (less than 1000 daltons), inorganic or organic compounds; and nucleic acid molecules including, but not limited to, double-stranded or single-stranded DNA, or double-stranded or single-stranded RNA (e.g., antisense, RNAi, etc.), aptamers, as well as triple helix nucleic acid molecules. Agents can be derived or obtained from any known organism (including, but not limited to, animals (e.g., mammals (human and non-human mammals)), plants, bacteria, fungi, and protista, or viruses) or from a library of synthetic molecules. Agents that are EphA2/EphrinA1 Modulators modulate (directly or indirectly): (i) the expression of EphA2 and/or an endogenous ligand(s) of EphA2, preferably, EphrinA1, at, e.g., the transcriptional, post-transcriptional, translational or post-translation level; and/or (ii) an activity(ies) of EphA2 and/or an endogenous ligand(s) of EphA2, preferably, EphrinA1.

As used herein, the term “analog” in the context of a proteinaceous agent (e.g., a peptide, polypeptide, protein or antibody) refers to a proteinaceous agent that possesses a similar or identical function as a second proteinaceous agent (e.g., an EphA2 polypeptide or an EphrinA1 polypeptide) but does not necessarily comprise a similar or identical amino acid sequence or structure of the second proteinaceous agent. A proteinaceous agent that has a similar amino acid sequence refers to a proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second proteinaceous agent; (b) a proteinaceous agent encoded by a nucleotide sequence that hybridizes under stringent conditions to a nucleotide sequence encoding a second proteinaceous agent of at least 20 amino acid residues, at least 30 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second proteinaceous agent. A proteinaceous agent with similar structure to a second proteinaceous agent refers to a proteinaceous agent that has a similar secondary, tertiary or quaternary structure of the second proteinaceous agent. The structure of a proteinaceous agent can be determined by methods known to those skilled in the art, including but not limited to, X-ray crystallography, nuclear magnetic resonance, and crystallographic electron microscopy. Preferably, the proteinaceous agent has EphA2 or EphrinA1 activity.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87: 2264-2268, modified as in Karlin and Altschul, 1993, Proc. Natl. Acad. Sci. U.S.A. 90: 5873-5877. Such an algorithm is incorporated into the NBLAST and XBLAST programs of Altschul et al., 1990, J. Mol. Biol. 215: 403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25: 3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4: 11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “analog” in the context of a non-proteinaceous analog refers to a second organic or inorganic molecule which possesses a similar or identical function as a first organic or inorganic molecule and is structurally similar to the first organic or inorganic molecule.

As used herein, the term “antibodies that immunospecifically bind to EphA2” and analogous terms refer to antibodies that specifically bind to an EphA2 polypeptide or a fragment of an EphA2 polypeptide, and do not specifically bind to non-EphA2 polypeptides. Preferably, antibodies that immunospecifically bind to an EphA2 polypeptide or a fragment thereof do not cross-react with other antigens. Antibodies that immunospecifically bind to an EphA2 polypeptide or a fragment thereof can be identified, for example, by immunoassays or other techniques known to those of skill in the art. Preferably, antibodies that immunospecifically bind to an EphA2 polypeptide or a fragment thereof only modulate an EphA2 activity(ies) and do not significantly affect other activities.

As used herein, the term “antibodies that immunospecifically bind to EphrinA1” and analogous terms refer to antibodies that specifically bind to an EphrinA1 polypeptide or a fragment of an EphrinA1 polypeptide, and do not specifically bind to non-EphrinA1 polypeptides. Preferably, antibodies that immunospecifically bind to an EphrinA1 polypeptide or a fragment thereof do not cross-react with other antigens. Antibodies that immunospecifically bind to an EphrinA1 polypeptide or a fragment thereof can be identified, for example, by immunoassays or other techniques known to those of skill in the art. Preferably, antibodies that immunospecifically bind to an EphrinA1 polypeptide or a fragment thereof only modulate an EphrinA1 activity(ies) and do not significantly affect other activities.

Antibodies of the invention include, but are not limited to, synthetic antibodies, monoclonal antibodies, recombinantly produced antibodies, multispecific antibodies (including bi-specific antibodies), human antibodies, humanized antibodies, chimeric antibodies, intrabodies, single-chain Fvs (scFv) (e.g., including monospecific and bi-specific, etc.), Fab fragments, F(ab′) fragments, disulfide-linked Fvs (sdFv), anti-idiotypic (anti-Id) antibodies, and epitope-binding fragments of any of the above. In particular, antibodies of the present invention include immunoglobulin molecules and immunologically active portions of immunoglobulin molecules, i.e., molecules that contain an antigen-binding site that immunospecifically binds to an EphA2 antigen or an EphrinA1 antigen (e.g., one or more complementarity determining regions (CDRs) of an anti-EphA2 antibody or of an anti-EphrinA1 antibody). The antibodies of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

As used herein, the term “cell proliferation stimulative” refers to the ability of proteinaceous molecules (including, but not limited to, peptides, polypeptides, proteins, post-translationally modified proteins, antibodies etc.), small molecules (less than 1000 daltons), inorganic or organic compounds, and nucleic acid molecules (including, but not limited to, double-stranded or single-stranded DNA, or double-stranded or single-stranded RNA (e.g., antisense, RNAi, etc.), aptamers, as well as triple helix nucleic acid molecules) to maintain, amplify, accelerate, or prolong cell proliferation, growth and/or survival in vivo or in vitro. Any method that detects cell proliferation, growth and/or survival, e.g., cell proliferation assays or epithelial barrier integrity assays, can be used to assay if an agent is a cell proliferation stimulative agent. Cell proliferation stimulative agents may also cause maintenance, regeneration, or reconstitution of epithelium when added to established colonies of hyperproliferative or damaged cells.

As used herein, the term “derivative” in the context of a proteinaceous agent (e.g., proteins, polypeptides, peptides, and antibodies) refers to a proteinaceous agent that comprises the amino acid sequence which has been altered by the introduction of amino acid residue substitutions, deletions, and/or additions. The term “derivative” as used herein also refers to a proteinaceous agent which has been modified, i.e., by the covalent attachment of a type of molecule to the proteinaceous agent. For example, but not by way of limitation, a derivative of a proteinaceous agent may be produced, e.g., by glycosylation, acetylation, pegylation, phosphorylation, amidation, derivatization by known protecting/blocking groups, proteolytic cleavage, linkage to a cellular ligand or other protein, etc. A derivative of a proteinaceous agent may also be produced by chemical modifications using techniques known to those of skill in the art, including, but not limited to specific chemical cleavage, acetylation, formylation, metabolic synthesis of tunicamycin, etc. Further, a derivative of a proteinaceous agent may contain one or more non-classical amino acids. A derivative of a proteinaceous agent possesses an identical function(s) as the proteinaceous agent from which it was derived. In a specific embodiment, a derivative of a proteinaceous agent is a derivative an EphA2 polypeptide, an EphrinA1 polypeptide, a fragment of an EphA2 polypeptide or EphrinA1 polypeptide, an antibody that immunospecifically binds to an EphA2 polypeptide or fragment thereof, or an antibody that immunospecifically binds to an EphrinA1 polypeptide or fragment thereof. In one embodiment, a derivative of an EphA2 polypeptide, an EphrinA1 polypeptide, a fragment of an EphA2 polypeptide or EphrinA1 polypeptide, an antibody that immunospecifically binds to an EphA2 polypeptide or fragment thereof, or an antibody that immunospecifically binds to an EphrinA1 polypeptide or fragment thereof possesses a similar or identical function as an EphA2 polypeptide, an EphrinA1 polypeptide, a fragment of an EphA2 polypeptide or EphrinA1 polypeptide, an antibody that immunospecifically binds to an EphA2 polypeptide or fragment thereof, or an antibody that immunospecifically binds to an EphrinA1 polypeptide or fragment thereof. In another embodiment, a derivative of an EphA2 polypeptide, an EphrinA1 polypeptide, a fragment of an EphA2 polypeptide or EphrinA1 polypeptide, an antibody that immunospecifically binds to an EphA2 polypeptide or fragment thereof, or an antibody that immunospecifically binds to an EphrinA1 polypeptide or fragment thereof has an altered activity when compared to an unaltered polypeptide. For example, a derivative antibody or fragment thereof can bind to its epitope more tightly or be more resistant to proteolysis.

As used herein, the term “derivative” in the context of a non-proteinaceous derivative refers to a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, carboxyl, nitryl, or amine group. An organic molecule may also, for example, be esterified, alkylated and/or phosphorylated.

As used herein, the term “effective amount” refers to the amount of a therapy (e.g., a prophylactic or therapeutic agent) which is sufficient to reduce and/or ameliorate the severity and/or duration of a non-neoplastic hyperproliferative epithelial and/or endothelial cell disorder or a symptom thereof, prevent the advancement of said disorder, cause regression of said disorder, prevent the recurrence, development, or onset of one or more symptoms associated with said disorder, or enhance or improve the prophylactic or therapeutic effect(s) of another therapy (e.g., prophylactic or therapeutic agent). Non-limiting examples of effective amounts of EphA2/EphrinA1 Modulators are provided in Section 4.7.3, infra.

As used herein, the term “endogenous ligand” or “natural ligand” refers to a molecule that normally binds a particular receptor in vivo. For example, EphrinA1 is an endogenous ligand of EphA2.

As used herein, the term “EphA2/EphrinA1 Modulator” refers to an agent(s) that confers a biological effect by modulating (directly or indirectly): (i) the expression of EphA2 and/or an endogenous ligand(s) of EphA2, preferably, EphrinA1, at, e.g., the transcriptional, post-transcriptional, translational or post-translation level; and/or (ii) an activity(ies) of EphA2 and/or an endogenous ligand(s) of EphA2, preferably, EphrinA1. Examples of EphA2/EphrinA1 Modulators include, but are not limited to, agents that inhibit or reduce the interaction between EphA2 and an endogenous ligand(s) of EphA2, preferably, EphrinA1 (hereinafter “EphA2/EphrinA1 Interaction Inhibitors”). Non-limiting examples of EphA2/EphrinA1 Interaction Inhibitors include: (i) agents that bind to EphA2, prevent or reduce the interaction between the EphA2 and EphrinA1, and induce EphA2 signal transduction (e.g., soluble forms of EphrinA1 (e.g., in monomeric or multimeric form), antibodies that bind EphA2, induce signaling and phosphorylation of EphA2 (i.e., an EphA2 agonistic antibody)); (ii) agents that bind to EphA2, prevent or reduce the interaction between the EphA2 and EphrinA1, and prevent or induce very low to negligible levels of EphA2 signal transduction (e.g., EphA2 antagonistic antibodies and dominant negative forms of EphrinA1); (iii) agents that bind to EphrinA1, prevent or reduce the interaction between an EphA2 and EphrinA1, and induce EphrinA1 signal transduction (e.g., soluble forms of EphA2 and antibodies that bind to EphrinA1 and induce EphrinA1 signal transduction); and (iv) agents that bind to EphrinA1, prevent or reduce the interaction between an EphA2 and EphrinA1, and prevent or induce very low to negligible levels of EphrinA1 signal transduction (e.g., dominant negative forms of EphA2 and anti-EphrinA1 antibodies).

In further embodiments, EphA2/EphrinA1 Modulators include, but are not limited to, agents that modulate the expression of EphA2. Such agents can decrease/downregulate EphA2 expression (e.g., EphA2 antisense molecules, RNAi and ribozymes) or increase/upregulate EphA2 expression such that the amount of EphA2 on the cell surface exceeds the amount of endogenous ligand (preferably, EphrinA1) available for binding, and thus, increases the amount of unbound EphA2 (e.g., nucleic acids encoding EphA2)).

In other embodiments, EphA2/EphrinA1 Modulators are agents that modulate the expression of EphrinA1. Such agents can decrease/downregulate EphrinA1 expression (e.g., EphrinA1 antisense molecules, RNAi and ribozymes) or increase/upregulate EphrinA1 expression (e.g., nucleic acids encoding EphrinA1)).

In yet other embodiments, EphA2/EphrinA1 Modulators of the invention include, but are not limited to, agents that modulate the protein stability or protein accumulation of EphA2 or EphrinA1. In a preferred embodiment, an EphA2 or Ephrin A1 Modulator of the invention increases protein stability and/or accumulation of EphA2.

In further embodiments, EphA2/EphrinA1 Modulators of the invention are agents that modulate kinase activity (e.g., of EphA2, EphrinA1 or of a heterologous protein known to associate with EphA2 or EphrinA1 at the cell membrane).

In further embodiments, EphA2/EphrinA1 Modulators of the invention include, but are not limited to, agents that bind to EphA2 and prevent or reduce EphA2 signal transduction but do not inhibit or reduce the interaction between EphA2 and EphrinA1 (e.g., an EphA2 intrabody); and agents that bind to EphrinA1 and prevent or reduce EphrinA1 signal transduction but do not inhibit or reduce the interaction between EphrinA1 and Eph EphA2 (e.g., an EphrinA1 antibody). In a preferred embodiment, EphA2/EphrinA1 Modulators of the invention decrease EphA2 cytoplasmic tail phosphorylation.

In a preferred embodiment of the invention, EphA2/EphrinA1 Modulators increase survival and/or growth of EphA2-expressing cells.

In another preferred embodiment of the invention, EphA2/EphrinA1 Modulators of the invention include, but are not limited to, dominant negative forms of EphA2; soluble forms of EphA2 (e.g., EphA2-Fc); Ephrin A1 antisense molecules; anti-EphA2 antibodies that bind to EphA2, interfere with EphA2-ligand interaction, and do not induce EphA2 signal transduction; and anti-EphrinA1 antibodies. In other embodiments, the anti-EphA2 and/or anti-EphrinA1 antibodies can be linked to a cytotoxic agent.

In a specific embodiment, an EphA2/EphrinA1 Modulator is not an agent that decreases the expression of EphA2. In another embodiment, an EphA2/EphrinA1 Modulator is not an agent that modulates the protein stability or protein accumulation of EphA2. In another embodiment, an EphA2/EphrinA1 Modulator is not an agent that modulates kinase activity (e.g., of EphA2, EphrinA1 or of a heterologous protein known to associate with EphA2 or EphrinA1 at the cell membrane). In another embodiment, an EphA2/EphrinA1 Modulator is not an EphA2 agonistic antibody. In a further embodiment, an EphA2/EphrinA1 Modulator is not an EphA2 antisense molecule. In yet a further embodiment, an EphA2/EphrinA1 Modulator is not a soluble form of EphrinA1 or a fragment thereof.

In a specific embodiment, an EphA2/EphrinA1 Modulator has one, two or all of the following cellular effects: (i) increases in the proliferation of EphA2-expressing cells; (ii) increases in the survival of EphA2 expressing cells (by, e.g., a preventing or reducing apoptosis and/or necrosis); and (iii) maintains and/or reconstitutes of the integrity of an epithelial and/or endothelial cell layer. In a particular embodiment, an EphA2/EphrinA1 Modulator prevents, reduces or slows the deposition of extracellular matrix (ECM) components (e.g., collagen, proteoglycans, tenascin and fibronectin). In another embodiment, an EphA2/EphrinA1 Modulator prevents, reduces or slows down angiogenesis. In another embodiment, an EphA2/EphrinA1 Modulator prevents, reduces or slows the deposition of extracellular matrix (ECM) components (e.g., collagen, proteoglycans, tenascin and fibronectin) and prevents, reduces or slows down angiogenesis.

As used herein, the term “EphA2 polypeptide” refers to EphA2, an analog, derivative or a fragment thereof, or a fusion protein comprising EphA2, an analog, derivative or a fragment thereof. The EphA2 polypeptide may be from any species. In certain embodiments, the term “EphA2 polypeptide” refers to the mature, processed form of EphA2. In other embodiments, the term “EphA2 polypeptide” refers to an immature form of EphA2. In accordance with this embodiment, the antibodies of the invention immunospecifically bind to the portion of the immature form of EphA2 that corresponds to the mature, processed form of EphA2.

The nucleotide and/or amino acid sequences of EphA2 polypeptides can be found in the literature or public databases, or the nucleotide and/or amino acid sequences can be determined using cloning and sequencing techniques known to one of skill in the art. For example, the nucleotide sequence of human EphA2 can be found in the GenBank database (see, e.g., Accession Nos. BC037166, M59371 and M36395). The amino acid sequence of human EphA2 can be found in the GenBank database (see, e.g., Accession Nos. AAH37166 and AAA53375). Additional non-limiting examples of amino acid sequences of EphA2 are listed in Table 1, infra.

TABLE 1

| Species | GenBank Accession No. |
|---|---|
| Mouse | NP_034269, AAH06954 |
| Rat | XP_345597 |

In a specific embodiment, a EphA2 polypeptide is EphA2 from any species. In a preferred embodiment, an EphA2 polypeptide is human EphA2.

As used herein, the term “EphrinA1 polypeptide” refers to EphrinA1, an analog, derivative or a fragment thereof, or a fusion protein comprising EphrinA1, an analog, derivative or a fragment thereof. The EphrinA1 polypeptide may be from any species. In certain embodiments, the term “EphrinA1 polypeptide” refers to the mature, processed form of EphrinA1. In other embodiments, the term “EphrinA1 polypeptide” refers to an immature form of EphrinA1. In accordance with this embodiment, the antibodies of the invention immunospecifically bind to the portion of the immature form of EphrinA1 that corresponds to the mature, processed form of EphrinA1.

The nucleotide and/or amino acid sequences of EphrinA1 polypeptides can be found in the literature or public databases, or the nucleotide and/or amino acid sequences can be determined using cloning and sequencing techniques known to one of skill in the art. For example, the nucleotide sequence of human EphrinA1 can be found in the GenBank database (see, e.g., Accession No. BC032698). The amino acid sequence of human EphrinA1 can be found in the GenBank database (see, e.g., Accession No. AAH32698). Additional non-limiting examples of amino acid sequences of EphrinA1 are listed in Table 2, infra.

TABLE 2

| Species | GenBank Accession No. |
|---|---|
| Mouse | NP_034237 |
| Rat | NP_446051 |

In a specific embodiment, a EphrinA1 polypeptide is EphrinA1 from any species. In a preferred embodiment, an EphrinA1 polypeptide is human EphrinA1.

As used herein, the term “epitope” refers to sites or fragments of a polypeptide or protein having antigenic or immunogenic activity in an animal, preferably in a mammal, and most preferably in a human. In specific embodiments, the term “epitope” refers to a portion of an EphA2 polypeptide or an EphrinA1 polypeptide having antigenic or immunogenic activity in an animal, preferably in a mammal, and most preferably in a human. An epitope having immunogenic activity is a site or fragment of a polypeptide or protein that elicits an antibody response in an animal. In specific embodiments, an epitope having immunogenic activity is a portion of an EphA2 polypeptide or an EphrinA1 polypeptide that elicits an antibody response in an animal. An epitope having antigenic activity is a site or fragment of a polypeptide or protein to which an antibody immunospecifically binds as determined by any method well-known to one of skill in the art, for example by immunoassays. In specific embodiments, an epitope having antigenic activity is a portion of an EphA2 polypeptide or an EphrinA1 polypeptide to which an antibody immunospecifically binds as determined by any method well known in the art, for example, by immunoassays. Antigenic epitopes need not necessarily be immunogenic.

As used herein, the term “fragment” in the context of a proteinaceous agent refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 30 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of another polypeptide or protein. In a specific embodiment, a fragment is a fragment of an EphA2 or EphrinA1 polypeptide, or an antibody that immunospecifically binds to an EphA2 or EphrinA1 polypeptide. In an embodiment, a fragment of a protein or polypeptide retains at least one function of the protein or polypeptide. In another embodiment, a fragment of a polypeptide or protein retains at least two, three, four, or five functions of the polypeptide or protein. Preferably, a fragment of an antibody that immunospecifically binds to an EphA2 polypeptide or fragment thereof, or an EphrinA1 polypeptide or fragment thereof retains the ability to immunospecifically bind to an EphA2 polypeptide or fragment thereof, or an EphrinA1 polypeptide or fragment thereof, respectively. Preferably, antibody fragments are epitope-binding fragments.

As used herein, the term “fusion protein” refers to a polypeptide or protein that comprises the amino acid sequence of a first polypeptide or protein or fragment, analog or derivative thereof, and the amino acid sequence of a heterologous polypeptide or protein (i.e., a second polypeptide or protein or fragment, analog or derivative thereof different than the first polypeptide or protein or fragment, analog or derivative thereof, or not normally part of the first polypeptide or protein or fragment, analog or derivative thereof). In one embodiment, a fusion protein comprises a prophylactic or therapeutic agent fused to a heterologous protein, polypeptide or peptide. In accordance with this embodiment, the heterologous protein, polypeptide or peptide may or may not be a different type of prophylactic or therapeutic agent. For example, two different proteins, polypeptides, or peptides with immunomodulatory activity may be fused together to form a fusion protein. In a preferred embodiment, fusion proteins retain or have improved activity relative to the activity of the original polypeptide or protein prior to being fused to a heterologous protein, polypeptide, or peptide.

As used herein, the term “humanized antibody” refers to forms of non-human (e.g., murine) antibodies, preferably chimeric antibodies, which contain minimal sequence derived from non-human immunoglobulin. For the most part, humanized antibodies are human immunoglobulins (recipient antibody) in which hypervariable region or complementarity determining (CDR) residues of the recipient are replaced by hypervariable region residues or CDR residues from an antibody from a non-human species (donor antibody) such as mouse, rat, rabbit or non-human primate having the desired specificity, affinity, and capacity. In some instances, one or more Framework Region (FR) residues of the human immunoglobulin are replaced by corresponding non-human residues or other residues based upon structural modeling, e.g., to improve affinity of the humanized antibody. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., 1986, Nature 321:522-525; Reichmann et al., 1988, Nature 332:323-329; Presta, 1992, Curr. Op. Struct. Biol. 2:593-596; and Queen et al., U.S. Pat. No. 5,585,089.

As used herein, the term “hybridizes under stringent conditions” describes conditions for hybridization and washing under which nucleotide sequences at least 30% (preferably, 35%, 40%, 45%, 50%, 55%, 60%, 65%, 70%, 75%, 80%, 85%, 90%, 95% or 98%) identical to each other typically remain hybridized to each other. Such stringent conditions are known to those skilled in the art and can be found in Current Protocols in Molecular Biology, John Wiley & Sons, N.Y. (1989), 6.3.1-6.3.6.

Generally, stringent conditions are selected to be about 5 to 10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (for example, 10 to 50 nucleotides) and at least about 60° C. for long probes (for example, greater than 50 nucleotides). Stringent conditions may also be achieved with the addition of destabilizing agents, for example, formamide. For selective or specific hybridization, a positive signal is at least two times background, preferably 10 times background hybridization.

In one, non-limiting example stringent hybridization conditions are hybridization at 6× sodium chloride/sodium citrate (SSC) at about 45° C., followed by one or more washes in 0.1×SSC, 0.2% SDS at about 68° C. In a preferred, non-limiting example stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

As used herein, the terms “hyperproliferative cell disorder” and “excessive cell accumulation disorder” refers to a disorder that is not neoplastic (i.e., non-neoplastic), in which cellular hyperproliferation or any form of excessive cell accumulation causes or contributes to the pathological state or symptoms of the disorder. In some embodiments, the hyperproliferative cell or excessive cell accumulation disorder is characterized by hyperproliferating epithelial cells. Hyperproliferative epithelial cell disorders include, but are not limited to, cirrhosis, fibrosis of the liver, kidney, lungs, heart, retina or other viscera, and fibrosis-related diseases. In other embodiments, the hyperproliferative cell or excessive cell accumulation disorder is characterized by hyperproliferating endothelial cells. In other embodiments, the hyperproliferative cell or excessive cell accumulation disorder is characterized by hyperproliferating fibroblasts. In yet other embodiments, the hyperproliferative cell or excessive cell accumulation disorder is characterized by aberrant angiogenesis. Disorders encompassed by the methods of the present invention that are associated with aberrant angiogenesis include, but are not limited to, asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis.

As used herein, the term “hypervariable region” refers to the amino acid residues of an antibody which are responsible for antigen binding. The hypervariable region comprises amino acid residues from a “Complementarity Determining Region” or “CDR” (i.e. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917). “Framework Region” or “FR” residues are those variable domain residues other than the hypervariable region residues as herein defined.

As used herein, the term “immunomodulatory agent” refers to an agent that modulates a subject's immune system. In particular, an immunomodulatory agent is an agent that alters the ability of a subject's immune system to respond to one or more foreign antigens. In a specific embodiment, an immunomodulatory agent is an agent that shifts one aspect of a subject's immune response. In a preferred embodiment of the invention, an immunomodulatory agent is an agent that inhibits or reduces a subject's immune response (i.e., an immunosuppressant agent). Preferably, an immunomodulatory agent that inhibits or reduces a subject's immune response inhibits or reduces the ability of a subject's immune system to respond to one or more foreign antigens. In certain embodiments, antibodies that immunospecifically bind IL-9 are immunomodulatory agents. In a specific embodiment, an immunomodulatory agent is an antibody that immunospecifically binds to CD2. Non-limiting examples of anti-CD2 antibodies include siplizumab (MedImmune, Inc., International Publication Nos. WO 02/098370 and WO 02/069904)). In another specific embodiment, an immunomodulatory agent is an agent that binds to αᵥβ₃ Non-limiting examples of antibodies that immunospecifically bind to integrin αᵥβ₃ include 11D2 (Searle), LM609 (Scripps), and VITAXIN™ (MedImmune, Inc.).

As used herein, the term “immunospecifically binds to EphA2” and analogous terms refers to peptides, polypeptides, proteins, fusion proteins, and antibodies or fragments thereof that specifically bind to an EphA2 receptor or one or more fragments thereof and do not specifically bind to other receptors or fragments thereof. The terms “immunospecifically binds to EphrinA1” and analogous terms refer to peptides, polypeptides, proteins, fusion proteins, and antibodies or fragments thereof that specifically bind to EphrinA1 or one or more fragments thereof and do not specifically bind to other ligands or fragments thereof. A peptide, polypeptide, protein, or antibody that immunospecifically binds to EphA2 or EphrinA1, or fragments thereof, may bind to other peptides, polypeptides, or proteins with lower affinity as determined by, e.g., immunoassays or other assays known in the art to detect binding affinity. Antibodies or fragments that immunospecifically bind to EphA2 or EphrinA1 may be cross-reactive with related antigens. Preferably, antibodies or fragments thereof that immunospecifically bind to EphA2 or EphrinA1 can be identified, for example, by immunoassays or other techniques known to those of skill in the art. An antibody or fragment thereof binds specifically to EphA2 or EphrinA1 when it binds to EphA2 or EphrinA1 with higher affinity than to any cross-reactive antigen as determined using experimental techniques, such as radioimmunoassays (RIAs) and enzyme-linked immunosorbent assays (ELISAs). See, e.g., Paul, ed., 1989, Fundamental Immunology, 2^{nd} ed., Raven Press, New York at pages 332-336 for a discussion regarding antibody specificity. In a preferred embodiment, an antibody that immunospecifically binds to EphA2 or EphrinA1 does not bind or cross-react with other antigens. In another embodiment, an antibody that binds to EphA2 or EphrinA1 that is a fusion protein specifically binds to the portion of the fusion protein that is EphA2 or EphrinA1.

As used herein, the term “in combination” refers to the use of more than one therapy. The use of the term “in combination” does not restrict the order in which therapies are administered to a subject with a non-neoplastic hyperproliferative epithelial and/or endothelial cell disorder. A first therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject which had, has, or is susceptible to a non-neoplastic hyperproliferative epithelial and/or endothelial cell disorder. Any additional therapy can be administered in any order with the other additional therapies. In certain embodiments, EphA2/EphrinA1 Modulators of the invention can be administered in combination with one or more therapies (e.g., non-EphA2/EphrinA1 Modulators currently administered to treat the disorder, analgesic agents, anesthetic agents, antibiotics, or immunomodulatory agents).

As used herein, the term “increased” with respect to the deposition of extracellular matrix (ECM) components (e.g., collagen, proteoglycans, tenascin and fibronectin) refers to an increase in the deposition of ECM components in an epithelial and/or endothelial cell layer of a subject with a non-neoplastic hyperproliferative epithelial and/or endothelial cell disorder associated with increased ECM components (e.g., cirrhosis and fibrosis (e.g., fibrosis of the liver, kidney, lungs, heart, retina and other viscera)) relative to the level of deposition of ECM components in an epithelial and/or endothelial cell layer of a normal, healthy subject and/or a population of normal, healthy cells. In a specific embodiment, the deposition of ECM components in an epithelial and/or endothelial cell layer in a subject with a non-neoplastic hyperproliferative epithelial and/or endothelial cell disorder is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% or at least 1.5 fold, at least 2 fold, at least 2.5 fold, at least 3 fold, at least 3.5 fold, at least 4 fold, at least 4.5, at least 5 fold, at least 7 fold or at least 10 fold relative to the level of deposition of ECM components in an epithelial and/or endothelial cell layer of a normal, healthy subject and/or a population of normal, healthy cells.

As used herein, the term “increased” with respect to angiogenesis refers to an increase in angiogenesis or angiogenic activity in a subject with a non-neoplastic hyperproliferative epithelial and/or endothelial cell disorder associated with aberrant angiogenesis or angiogenic activity (e.g., asthma, ischemia, atherosclerosis, diabetic retinopathy, macular degeneration, rheumatoid arthritis, osteoarthritis and psoriasis) relative to the level of angiogenesis or angiogenic activity in normal, healthy subject and/or a population of normal, healthy cells. In a specific embodiment, the level of angiogenesis or angiogenic activity in a subject with a non-neoplastic hyperproliferative epithelial and/or endothelial cell disorder is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% or at least 1.5 fold, at least 2 fold, at least 2.5 fold, at least 3 fold, at least 3.5 fold, at least 4 fold, at least 4.5, at least 5 fold, at least 7 fold or at least 10 fold relative to the level of angiogenesis or angiogenic activity in a normal, healthy subject and/or a population of normal, healthy cells.

As used herein, the term “isolated” in the context of an organic or inorganic molecule (whether it be a small or large molecule), other than a proteinaceous agent or a nucleic acid, refers to an organic or inorganic molecule substantially free of a different organic or inorganic molecule. Preferably, an organic or inorganic molecule is 60%, 65%, 70%, 75%, 80%, 85%, 90%, 95%, or 99% free of a second, different organic or inorganic molecule. In a preferred embodiment, an organic and/or inorganic molecule is isolated.

As used herein, the term “isolated” in the context of a proteinaceous agent (e.g., a peptide, polypeptide, fusion protein, or antibody) refers to a proteinaceous agent which is substantially free of cellular material or contaminating proteins from the cell or tissue source from which it is derived, or substantially free of chemical precursors or other chemicals when chemically synthesized. The language “substantially free of cellular material” includes preparations of a proteinaceous agent in which the proteinaceous agent is separated from cellular components of the cells from which it is isolated or recombinantly produced. Thus, a proteinaceous agent that is substantially free of cellular material includes preparations of a proteinaceous agent having less than about 30%, 20%, 10%, or 5% (by dry weight) of heterologous protein, polypeptide, peptide, or antibody (also referred to as a “contaminating protein”). When the proteinaceous agent is recombinantly produced, it is also preferably substantially free of culture medium, i.e., culture medium represents less than about 20%, 10%, or 5% of the volume of the proteinaceous agent preparation. When the proteinaceous agent is produced by chemical synthesis, it is preferably substantially free of chemical precursors or other chemicals, i.e., it is separated from chemical precursors or other chemicals which are involved in the synthesis of the proteinaceous agent. Accordingly, such preparations of a proteinaceous agent have less than about 30%, 20%, 10%, 5% (by dry weight) of chemical precursors or compounds other than the proteinaceous agent of interest. In a specific embodiment, proteinaceous agents disclosed herein are isolated. In a preferred embodiment, a proteinaceous EphA2/EphrinA1 Modulator of the invention is isolated.

As used herein, the term “isolated” in the context of nucleic acid molecules refers to a nucleic acid molecule which is separated from other nucleic acid molecules which are present in the natural source of the nucleic acid molecule. Moreover, an “isolated” nucleic acid molecule, such as a cDNA molecule, is preferably substantially free of other cellular material, or culture medium when produced by recombinant techniques, or substantially free of chemical precursors or other chemicals when chemically synthesized. In a specific embodiment, nucleic acid molecules are isolated. In a preferred embodiment, an EphA2/EphrinA1 Modulator that is a nucleic acid molecule is isolated.

As used herein, the term “low tolerance” refers to a state in which the patient suffers from side effects from treatment so that the patient does not benefit from and/or will not continue therapy because of the adverse effects and/or the harm from side effects outweighs the benefit of the treatment.

As used herein, the terms “manage”, “managing” and “management” refer to the beneficial effects that a subject derives from a therapy, which does not result in a cure of the disorder. In certain embodiments, a subject is administered one or more therapies to “manage” a disorder so as to prevent the progression or worsening of the disorder (i.e., hold disease progress).

As used herein, the term “neoplastic” refers to a disease involving cells that have the potential to metastasize to distal sites and exhibit phenotypic traits that differ from those of non-neoplastic cells, for example, formation of colonies in a three-dimensional substrate such as soft agar or the formation of tubular networks or weblike matrices in a three-dimensional basement membrane or extracellular matrix preparation, such as MATRIGEL™. Non-neoplastic cells do not form colonies in soft agar and form distinct sphere-like structures in three-dimensional basement membrane or extracellular matrix preparations. Neoplastic cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless replicative potential, and sustained angiogenesis. Thus, “non-neoplastic” means that the condition, disease, or disorder does not involve cancer cells.

As used herein, the term “pathology-causing cell phenotype” or “pathology-causing epithelial and/or endothelial cell phenotype” refers to a function that a non-neoplastic hyperproliferating epithelial and/or endothelial cell performs that causes or contributes to the pathological state of a non-neoplastic epithelial and/or endothelial hyperproliferative disorder. Pathology-causing epithelial cell phenotypes include secretion of mucin, differentiation into a mucin-secreting cell, secretion of inflammatory factors, and hyperproliferation. Pathology-causing endothelial cell phenotypes include increased cell migration (not including metastasis), increased cell volume, secretion of extracellular matrix molecules (e.g., collagen, tenascin fibronectin, proteoglycans, etc.) or matrix metalloproteinases (e.g., gelatinases, collagenases, and stromelysins), hyperproliferation, and/or aberrant angiogenesis. One or more of these pathology-causing cell phenotypes causes or contributes to symptoms in a patient suffering from a non-neoplastic hyperproliferative epithelial and/or endothelial cell disorder such as cirrhosis, fibrosis (e.g., fibrosis of the liver, kidney, lungs, heart, retina or other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the term “potentiate” refers to an improvement in the efficacy of a therapy at its common or approved dose.

As used herein, the terms “prevent,” “preventing,” and “prevention” refer to the inhibition of the development or onset of a non-neoplastic hyperproliferative epithelial and/or endothelial disorder or the prevention of the recurrence, onset, or development of one or more symptoms of a non-neoplastic hyperproliferative epithelial and/or endothelial disorder in a subject resulting from the administration of a therapy (e.g., a prophylactic or therapeutic agent), or the administration of a combination of therapies (e.g., a combination of prophylactic or therapeutic agents).

As used herein, the term “prophylactic agent” refers to any agent that can prevent the recurrence, spread or onset of a non-neoplastic hyperproliferative epithelial and/or endothelial cell disorder, such as cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis, or a symptom thereof. In certain embodiments, the term “prophylactic agent” refers to an EphA2/EphrinA1 Modulator. In certain other embodiments, the term “prophylactic agent” refers to an agent other than an EphA2/EphrinA1 Modulator. Preferably, a prophylactic agent is an agent which is known to be useful to or has been or is currently being used to the prevent or impede the onset, development, progression and/or severity of a non-neoplastic hyperproliferative epithelial and/or endothelial cell disorder or one or more symptoms thereof.

As used herein, a “prophylactically effective amount” refers to that amount of a therapy (e.g., a prophylactic agent) sufficient to result in the prevention of the recurrence, spread or onset of a non-neoplastic hyperproliferative epithelial and/or endothelial cell disorder (including, but not limited to cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis) or a symptom thereof. A prophylactically effective amount may refer to the amount of a therapy (e.g., a prophylactic agent) sufficient to prevent the occurrence, spread or recurrence of a non-neoplastic hyperproliferative epithelial and/or endothelial cell disorder in patients predisposed to a non-neoplastic hyperproliferative cell disorder, for example those genetically predisposed or those having previously suffered from such a disorder. A prophylactically effective amount may also refer to the amount of a therapy (e.g., a prophylactic agent) that provides a prophylactic benefit in the prevention of a non-neoplastic hyperproliferative epithelial and/or endothelial cell disorder such as cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis. Further, a prophylactically effective amount with respect to a therapy (e.g., a prophylactic agent of the invention) means that amount of the therapy (e.g., prophylactic agent) alone, or in combination with one or more other therapies (e.g., non-EphA2/EphrinA1 Modulators currently administered to prevent the disorder, analgesic agents, anesthetic agents, antibiotics, or immunomodulatory agents) that provides a prophylactic benefit in the prevention of a non-neoplastic hyperproliferative epithelial and/or endothelial cell disorder such as cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis. Used in connection with an amount of an EphA2/EphrinA1 Modulator of the invention, the term can encompass an amount that improves overall prophylaxis or enhances the prophylactic efficacy of or synergies with another therapy, (e.g., a prophylactic agent).

A used herein, a “protocol” includes dosing schedules and dosing regimens.

As used herein, the term “refractory” refers to a non-neoplastic hyperproliferative epithelial and/or endothelial cell disorder such as cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis, that is not responsive to one or more therapies (e.g., currently available therapies). In a certain embodiment, that a non-neoplastic hyperproliferative epithelial and/or endothelial cell disorder such as cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis is refractory to a therapy means that at least some significant portion of the symptoms associated with the disorder are not eliminated or lessened by that therapy. The determination of whether a non-neoplastic hyperproliferative epithelial and/or cell disorder such as cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis, is refractory can be made either in vivo or in vitro by any method known in the art for assaying the effectiveness of therapy for a non-neoplastic hyperproliferative epithelial and/or cell disorder such as cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis.

As used herein, the phrase “side effects” encompasses unwanted and adverse effects of a therapy (e.g., a prophylactic or therapeutic agent). Adverse effects are always unwanted, but unwanted effects are not necessarily adverse. An adverse effect from a therapy (e.g., a prophylactic or therapeutic agent) might be harmful or uncomfortable or risky. Examples of side effects include, but are not limited to, nausea, vomiting, anorexia, abdominal cramping, fever, pain, loss of body weight, dehydration, alopecia, dyspnea, insomnia, dizziness, mucositis, nerve and muscle effects, fatigue, dry mouth, and loss of appetite, rashes or swellings at the site of administration, flu-like symptoms such as fever, chills and fatigue, digestive tract problems and allergic reactions. Additional undesired effects experienced by patients are numerous and known in the art. Many are described in the Physicians' Desk Reference (58^{th} ed., 2004).

As used herein, the term “single-chain Fv” or “scFv” refers to antibody fragments comprising the V_{H} and V_{L} domains of antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv see Pluckthun in The Pharmacology of Monoclonal Antibodies, vol. 113, Rosenburg and Moore eds. Springer-Verlag, New York, pp. 269-315 (1994).

As used herein, the terms “subject” and “patient” are used interchangeably. As used herein, a subject is preferably a mammal such as a non-primate (e.g., cows, pigs, horses, cats, dogs, rats etc.) and a primate (e.g., monkey and human), most preferably a human. In one embodiment, the subject is a mammal, preferably a human, with a non-neoplastic hyperproliferative epithelial and/or endothelial cell disorder. In another embodiment, the subject is a farm animal (e.g., a horse, pig, or cow), a pet (e.g., a guinea pig, dog or cat), or a laboratory animal (e.g., an animal model) with a non-neoplastic hyperproliferative epithelial and/or endothelial cell disorder. In another embodiment, the subject is a mammal, preferably a human, at risk of developing a non-neoplastic hyperproliferative epithelial and/or endothelial cell disorder (e.g., an immunocompromised or immunosuppressed mammal, or a genetically predisposed mammal). In another embodiment, the subject is not an immunocompromised or immunosuppressed mammal, preferably a human. In another embodiment, the subject is a mammal, preferably a human, with a lymphocyte count that is not under approximately 500 cells/mm³.

As used herein, the term “synergistic” refers to a combination of therapies (e.g., prophylactic or therapeutic agents) which is more effective than the additive effects of any two or more single therapies (e.g., one or more prophylactic or therapeutic agents). A synergistic effect of a combination of therapies (e.g., a combination of prophylactic or therapeutic agents) permits the use of lower dosages of one or more of therapies (e.g., one or more prophylactic or therapeutic agents) and/or less frequent administration of said therapies to a subject with a non-neoplastic hyperproliferative epithelial and/or endothelial cell disorder. The ability to utilize lower dosages of therapies (e.g., prophylactic or therapeutic agents) and/or to administer said therapies less frequently reduces the toxicity associated with the administration of said therapies to a subject without reducing the efficacy of said therapies in the prevention or treatment of a non-neoplastic hyperproliferative epithelial and/or endothelial cell disorder. In addition, a synergistic effect can result in improved efficacy of therapies (e.g., prophylactic or therapeutic agents) in the prevention or treatment of a non-neoplastic hyperproliferative epithelial and/or endothelial cell disorder. Finally, synergistic effect of a combination of therapies (e.g., prophylactic or therapeutic agents) may avoid or reduce adverse or unwanted side effects associated with the use of any single therapy.

As used herein, the term “therapeutic agent” refers to any agent that can be used in the treatment, management, prevention, or symptom reduction of a non-neoplastic hyperproliferative epithelial and/or endothelial cell disorder such as cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis. In certain embodiments, the term “therapeutic agent” refers to an EphA2/EphrinA1 Modulator. In certain other embodiments, the term “therapeutic agent” refers an agent other than an EphA2/EphrinA1 Modulator. Preferably, a therapeutic agent is an agent which is known to be useful for, or has been or is currently being used for the prevention, treatment, management, or amelioration of a non-neoplastic epithelial and/or endothelial cell disorder or one or more symptoms thereof.

As used herein, a “therapeutically effective amount” refers to that amount of a therapy (e.g., a therapeutic agent) sufficient to treat, manage, or ameliorate a non-neoplastic hyperproliferative epithelial and/or endothelial cell disorder (such as cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis), ameliorate one or more symptoms of a d non-neoplastic hyperproliferative epithelial and/or endothelial cell disorder, delay or minimize the onset or severity of the non-neoplastic hyperproliferative epithelial and/or endothelial cell disorder, prevent the advancement of a non-neoplastic hyperproliferative epithelial and/or endothelial cell disorder, cause regression of a non-neoplastic hyperproliferative epithelial and/or endothelial cell disorder, enhance or improve the therapeutic effect(s) of another therapy, or provide a therapeutic benefit in the treatment or management of a non-neoplastic hyperproliferative epithelial and/or endothelial cell disorder. Preferably, “a therapeutically effective amount” is an amount sufficient to eliminate, modify, or control symptoms associated with a non-neoplastic hyperproliferative epithelial and/or endothelial cell disorder. Used in connection with an amount of an EphA2/EphrinA1 Modulator of the invention, the term can encompass an amount that improves overall therapeutic effect, reduces or avoids unwanted effects, or enhances the therapeutic efficacy of or synergies with another therapy.

As used herein, the term “therapy” refers to any protocol, method and/or agent that can be used in the prevention, treatment or management of a non-neoplastic hyperproliferative epithelial and/or endothelial cell disorder such as cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis. In certain embodiments, the terms “therapies” and “therapy” refer to a biological therapy, supportive therapy, and/or other therapies useful the in treatment, management, prevention, or amelioration of a non-neoplastic hyperproliferative epithelial and/or endothelial cell disorder or one or more symptoms thereof known to one of skill in the art such as medical personnel.

As used herein, the terms “treat”, “treating” and “treatment” refer to the eradication, reduction or amelioration of symptoms of a disorder, particularly, the eradication, removal, modification, or control of a non-neoplastic hyperproliferative epithelial and/or endothelial cell disorder such as cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis that results from the administration of one or more therapies (e.g., prophylactic or therapeutic agents). In certain embodiments, such terms refer to the minimizing or delay of the spread of the non-neoplastic hyperproliferative epithelial and/or endothelial cell disorder such as cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis resulting from the administration of one or more therapies (e.g., prophylactic or therapeutic agents) to a subject with such a disorder.

### 4. DETAILED DESCRIPTION OF THE INVENTION

The present invention provides methods for the prevention, management, treatment and/or amelioration of a non-neoplastic hyperproliferative epithelial cell and/or endothelial cell disorder (including, but not limited to, a disorder associated with increased deposition of extracellular matrix (ECM) components and a disorder associated with aberrant angiogenesis) or a symptom thereof, the methods comprising administering to a subject in need thereof an effective amount of an EphA2/EphrinA1 Modulator. The present invention also provides methods for the prevention, management, treatment and/or amelioration of a non-neoplastic hyperproliferative epithelial cell and/or endothelial cell disorder (including, but not limited to, a disorder associated with increased deposition of extracellular matrix (ECM) components and a disorder associated with increased or aberrant angiogenesis) or a symptom thereof, the methods comprising administering to a subject in need thereof an effective amount of an EphA2/EphrinA1 Modulator and an effective amount of a therapy other than an EphA2/EphrinA1 Modulator (e.g., an analgesic agent, an anesthetic agent, an antibiotic, or an immunomodulatory agent). Non-limiting examples of EphA2/EphrinA1 Modulators include, but are not limited to, agents that inhibit or reduce the interaction between EphA2 and an endogenous ligand(s) of EphA2, preferably, EphrinA1 (hereinafter “EphA2/EphrinA1 Interaction Inhibitors”). Non-limiting examples of EphA2/EphrinA1 Interaction Inhibitors include: (i) agents that bind to EphA2, prevent or reduce the interaction between the EphA2 and EphrinA1, and induce EphA2 signal transduction (e.g., soluble forms of EphrinA1 (e.g., in monomeric or multimeric form), antibodies that bind EphA2, induce signaling and phosphorylation of EphA2 (i.e., an EphA2 agonistic antibody)); (ii) agents that bind to EphA2, prevent or reduce the interaction between the EphA2 and EphrinA1, and prevent or induce very low to negligible levels of EphA2 signal transduction (e.g., EphA2 antagonistic antibodies and dominant negative forms of EphrinA1); (iii) agents that bind to EphrinA1, prevent or reduce the interaction between an EphA2 and EphrinA1, and induce EphrinA1 signal transduction (e.g., soluble forms of EphA2 and antibodies that bind to EphrinA1 and induce EphrinA1 signal transduction); and (iv) agents that bind to EphrinA1, prevent or reduce the interaction between an EphA2 and EphrinA1, and prevent or induce very low to negligible levels of EphrinA1 signal transduction (e.g., dominant negative forms of EphA2 and anti-EphrinA1 antibodies).

In further embodiments, EphA2/EphrinA1 Modulators include, but are not limited to, agents that modulate the expression of EphA2. Such agents can decrease/downregulate EphA2 expression (e.g., EphA2 antisense molecules, RNAi and ribozymes) or increase/upregulate EphA2 expression such that the amount of EphA2 on the cell surface exceeds the amount of endogenous ligand (preferably, EphrinA1) available for binding, and thus, increases the amount of unbound EphA2 (e.g., nucleic acids encoding EphA2)).

In other embodiments, EphA2/EphrinA1 Modulators are agents that modulate the expression of EphrinA1. Such agents can decrease/downregulate EphrinA1 expression (e.g., EphrinA1 antisense molecules, RNAi and ribozymes) or increase/upregulate EphrinA1 expression (e.g., nucleic acids encoding EphrinA1)).

In yet other embodiments, EphA2/EphrinA1 Modulators of the invention include, but are not limited to, agents that modulate the protein stability or protein accumulation of EphA2 or EphrinA1. In a preferred embodiment, an EphA2 or Ephrin A1 Modulator of the invention increases protein stability and/or accumulation of EphA2.

In further embodiments, EphA2/EphrinA1 Modulators of the invention are agents that modulate kinase activity (e.g., of EphA2, EphrinA1 or of a heterologous protein known to associate with EphA2 or EphrinA1 at the cell membrane).

In further embodiments, EphA2/EphrinA1 Modulators of the invention include, but are not limited to, agents that bind to EphA2 and prevent or reduce EphA2 signal transduction but do not inhibit or reduce the interaction between EphA2 and EphrinA1 (e.g., an EphA2 intrabody); and agents that bind to EphrinA1 and prevent or reduce EphrinA1 signal transduction but do not inhibit or reduce the interaction between EphrinA1 and Eph EphA2 (e.g., an EphrinA1 antibody). In a preferred embodiment, EphA2/EphrinA1 Modulators of the invention decrease EphA2 cytoplasmic tail phosphorylation.

In a preferred embodiment of the invention, EphA2/EphrinA1 Modulators increase survival and/or growth of EphA2-expressing cells.

In another preferred embodiment of the invention, EphA2/EphrinA1 Modulators of the invention include, but are not limited to, dominant negative forms of EphA2; soluble forms of EphA2 (e.g., EphA2-Fc); Ephrin A1 antisense molecules; anti-EphA2 monoclonal antibodies that bind to EphA2, interfere with EphA2-ligand interaction, and do not induce EphA2 signal transduction; and anti-EphrinA1 monoclonal antibodies. In other embodiments, the anti-EphrinA1 monoclonal antibodies can be linked to a cytotoxic agent.

In a specific embodiment, an EphA2/EphrinA1 Modulator is not an agent that decreases the expression of EphA2. In another embodiment of the invention, an EphA2/EphrinA1 Modulator is not an agent that modulates kinase activity (e.g., of EphA2, EphrinA1 or of a heterologous protein known to associate with EphA2 or EphrinA1 at the cell membrane). In another embodiment, an EphA2/EphrinA1 Modulator is not an agent that modulates protein stability or protein accumulation of EphA2. In another embodiment, an EphA2/EphrinA1 Modulator is not an EphA2 agonistic antibody. In a further embodiment, an EphA2/EphrinA1 Modulator is not an EphA2 antisense molecule. In yet a further embodiment, an EphA2/EphrinA1 Modulator is not a soluble form of EphrinA1 or a fragment thereof.

The present invention provides methods for the screening and identification of EphA2/EphrinA1 Modulators that modulate (e.g., increase or decrease the expression and/or activity) EphA2 and/or EphrinA1, e.g., decrease EphA2-endogenous ligand binding, decrease EphrinA1 gene expression, upregulate EphA2 gene expression, increase EphA2 protein stability or protein accumulation, decrease EphA2 cytoplasmic tail phosphorylation, increase proliferation of EphA2 expressing cells, increase survival of EphA2 expressing cells (e.g., by preventing apoptosis), maintain/reconstitute the integrity of an epithelial and/or endothelial cell layer, and/or prevent or slow angiogenesis. In a specific embodiment, the invention provides methods for screening and identifying EphA2/EphrinA1 Modulators that prevent and/or slow the progression of non-neoplastic hyperproliferative epithelial and/or endothelial cell disorders such as cirrhosis, fibrosis (e.g., fibrosis of the liver, kidney, lungs, heart, retina and other viscera) by preventing or slowing the deposition of ECM components (e.g., collagen) in the epithelial and/or endothelial cell layers.

In one embodiment, the invention provides methods for screening and identifying EphA2/EphrinA1 Modulators that prevent and/or slow the progression of non-neoplastic hyperproliferative epithelial and/or endothelial cell disorders by preventing, reducing or slowing down angiogenesis. In an alternative embodiment, the invention provides methods for screening and identifying EphA2/EphrinA1 Modulators that prevent and/or slow the progression of non-neoplastic hyperproliferative epithelial and/or endothelial cell disorders by increasing angiogenisis.

The present invention provides pharmaceutical compositions and prophylactic and therapeutic regimens designed to treat, manage, or prevent non-neoplastic hyperproliferative epithelial and/or endothelial cell disorders such as cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis. In a specific embodiment, the present invention provides pharmaceutical compositions and prophylactic and therapeutic regimens that prevent or slow down the deposition of ECM components (e.g., collagen) in the epithelial and/or endothelial cell layers, and/or modulate angiogenesis, and the use of such compositions and regimens in the treatment, management or prevention of non-neoplastic hyperproliferative epithelial cell disorders, in particular fibrosis and/or fibrosis-related diseases.

In a specific embodiment, the present invention provides pharmaceutical compositions and prophylactic and therapeutic regimens designed to decrease angiogenesis. In another embodiment, the invention provides pharmaceutical compositions and prophylactic and therapeutic regimens designed to increase angiogenesis.

The invention further provides diagnostic methods using the EphA2/EphrinA1 Modulators of the invention to evaluate the efficacy of a therapy for a non-neoplastic hyperproliferative epithelial and/or endothelial cell disorder (e.g., cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis), wherein the therapy monitored can be either EphA2/EphrinA1-based or not EphA2/EphrinA1-based. In particular embodiments, the diagnostic methods of the invention provide methods of imaging areas of hyperproliferation. The diagnostic methods of the invention may also be used to prognose or predict non-neoplastic hyperproliferative epithelial and/or endothelial cell disorders (e.g., cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis). The EphA2/EphrinA1 Modulators of the invention may also be used for immunohistochemical analyses of frozen or fixed cells or tissue assays.

The invention also provides kits comprising the pharmaceutical compositions or diagnostic reagents of the invention.

4.1 EphA2/EphrinA1 Modulators

The invention provides modulators of EphA2 and/or EphrinA1 (“EphA2/EphrinA1 Modulators”). Non-limiting examples of EphA2/EphrinA1 Modulators are agents that confer a biological effect by modulating (directly or indirectly): (i) the expression of EphA2 and/or an endogenous ligand(s) of EphA2 (preferably, EphrinA1), at, e.g., the transcriptional, post-transcriptional, translational or post-translation level; and/or (ii) an activity(ies) of EphrinA1.

Examples of EphA2/EphrinA1 Modulators include, but are not limited to, agents that inhibit or reduce the interaction between EphA2 and an endogenous ligand(s) of EphA2, preferably, EphrinA1 (hereinafter “EphA2/EphrinA1 Interaction Inhibitors”). Non-limiting examples of EphA2/EphrinA1 Interaction Inhibitors include: (i) agents that bind to EphA2, prevent or reduce the interaction between the EphA2 and EphrinA1, and induce EphA2 signal transduction (e.g., soluble forms of EphrinA1 (e.g., in monomeric or multimeric form), antibodies that bind EphA2, induce signaling and phosphorylation of EphA2 (i.e., an EphA2 agonistic antibody)); (ii) agents that bind to EphA2, prevent or reduce the interaction between the EphA2 and EphrinA1, and prevent or induce very low to negligible levels of EphA2 signal transduction (e.g., EphA2 antagonistic antibodies and dominant negative forms of EphrinA1); (iii) agents that bind to EphrinA1, prevent or reduce the interaction between an EphA2 and EphrinA1, and induce EphrinA1 signal transduction (e.g., soluble forms of EphA2 and antibodies that bind to EphrinA1 and induce EphrinA1 signal transduction); and (iv) agents that bind to EphrinA1, prevent or reduce the interaction between an EphA2 and EphrinA1, and prevent or induce very low to negligible levels of EphrinA1 signal transduction (e.g., dominant negative forms of EphA2 and anti-EphrinA1 antibodies).

In further embodiments, EphA2/EphrinA1 Modulators include, but are not limited to, agents that modulate the expression of EphA2. Such agents can decrease/downregulate EphA2 expression (e.g., EphA2 antisense molecules, RNAi and ribozymes) or increase/upregulate EphA2 expression such that the amount of EphA2 on the cell surface exceeds the amount of endogenous ligand (preferably, EphrinA1) available for binding, and thus, increases the amount of unbound EphA2 (e.g.; nucleic acids encoding EphA2)).

In other embodiments, EphA2/EphrinA1 Modulators are agents that modulate the expression of EphrinA1. Such agents can decrease/downregulate EphrinA1 expression (e.g., EphrinA1 antisense molecules, RNAi and ribozymes) or increase/upregulate EphrinA1 expression (e.g., nucleic acids encoding EphrinA1)).

In yet other embodiments, EphA2/EphrinA1 Modulators of the invention include, but are not limited to, agents that modulate the protein stability or protein accumulation of EphA2 or EphrinA1. In a preferred embodiment, an EphA2 or Ephrin A1 Modulator of the invention increases protein stability and/or accumulation of EphA2.

In further embodiments, EphA2/EphrinA1 Modulators of the invention are agents that modulate kinase activity (e.g., of EphA2, EphrinA1 or of a heterologous protein known to associate with EphA2 or EphrinA1 at the cell membrane).

In further embodiments, EphA2/EphrinA1 Modulators of the invention include, but are not limited to, agents that bind to EphA2 and prevent or reduce EphA2 signal transduction but do not inhibit or reduce the interaction between EphA2 and EphrinA1 (e.g., an EphA2 intrabody); and agents that bind to EphrinA1 and prevent or reduce EphrinA1 signal transduction but do not inhibit or reduce the interaction between EphrinA1 and Eph EphA2 (e.g., an EphrinA1 antibody). In a preferred embodiment, EphA2/EphrinA1 Modulators of the invention decrease EphA2 cytoplasmic tail phosphorylation.

In a preferred embodiment of the invention, EphA2/EphrinA1 Modulators increase survival and/or growth of EphA2-expressing cells.

In another preferred embodiment of the invention, EphA2/EphrinA1 Modulators of the invention include, but are not limited to, dominant negative forms of EphA2; soluble forms of EphA2 (e.g., EphA2-Fc); Ephrin A1 antisense molecules; anti-EphA2 monoclonal antibodies that bind to EphA2, interfere with EphA2-ligand interaction, and do not induce EphA2 signal transduction; and anti-EphrinA1 monoclonal antibodies. In other embodiments, the anti-EphrinA1 monoclonal antibodies can be linked to a cytotoxic agent.

In a specific embodiment, an EphA2/EphrinA1 Modulator is not an agent that decreases the expression of EphA2. In another embodiment, an EphA2/EphrinA1 Modulator is not an agent that modulates the protein stability or protein accumulation of EphA2. In another embodiment of the invention, an EphA2/EphrinA1 Modulator is not an agent that modulates kinase activity (e.g., of EphA2, EphrinA1 or of a heterologous protein known to associate with EphA2 or EphrinA1 at the cell membrane). In another embodiment, an EphA2/EphrinA1 Modulator is not an EphA2 agonistic antibody. In a further embodiment, an EphA2/EphrinA1 Modulator is not an EphA2 antisense molecule. In yet a further embodiment, an EphA2/EphrinA1 Modulator is not a soluble form of EphrinA1 or a fragment thereof.

In a specific embodiment, an EphA2/EphrinA1 Modulator is an agent that decreases or downregulates EphA2 expression (e.g., EphA2 antisense molecules, RNAi and ribozymes). In a particular embodiment, the EphA2/EphrinA1 Modulator decreases or downregulates EphA2 expression by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline) in an assay described herein or known in the art (e.g., RT-PCR, a Northern blot or an immunoassay such as an ELISA). In alternative embodiment, an EphA2/EphrinA1 Modulator is an agent that increases or upregulates the expression of EphA2 such that the amount of EphA2 on the cell surface exceeds the amount of endogenous ligand (preferably, EphrinA1) available for binding, and thus, increases the amount of unbound EphA2 (e.g., nucleic acids encoding EphA2)). In a particular embodiment, the EphA2/EphrinA1 Modulator increases or upregulates EphA2 expression by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline) in an assay described herein or known in the art (e.g., RT-PCR, a Northern blot or an immunoassay such as an ELISA).

In a specific embodiment, an EphA2/EphrinA1 Modulator is an agent that reduces the protein stability and/or protein accumulation of EphA2. In another embodiment, the EphA2/EphrinA1 Modulator reduces the protein stability and/or protein accumulation of EphA2 expression by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline) in an assay described herein or known in the art (e.g., an immunoassay). In an alternative embodiment, an EphA2/EphrinA1 Modulator is an agent that increases the protein stability and/or protein accumulation of EphA2. In a further embodiment, the EphA2/EphrinA1 Modulator increases the protein stability and/or protein accumulation of EphA2 expression by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline) in an assay described herein or known in the art (e.g., an immunoassay).

In a specific embodiment, an EphA2/EphrinA1 Modulator is an agent that inhibits or decreases the expression of EphrinA1 (e.g., EphrinA1 antisense molecules, RNAi and ribozymes). In a particular embodiment, the EphA2/EphrinA1 Modulator decreases the expression of EphrinA1 by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline) in an assay described herein or known in the art (e.g., RT-PCR, a Northern blot or an immunoassay such as an ELISA).

In another embodiment, an EphA2/EphrinA1 Modulator is an agent that binds to EphA2 and prevents or reduces EphA2 signal transduction but does not inhibit or reduce the interaction between EphA2 and an endogenous ligand(s) of EphA2, preferably, EphrinA1 (e.g., an EphA2 intrabody). In a particular embodiment, the EphA2/EphrinA1 Modulator reduces EphA2 signal transduction by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline) in an assay described herein or known in the art (e.g., an immunoassay). In accordance with this embodiment, the EphA2/EphrinA1 Modulator does not reduce or only reduces the interaction between EphA2 and an endogenous ligand(s) of EphA2 (preferably, EphrinA1) by 5% or less, 10% or less, 15% or less, 20% or less, 25% or less, 30% or less, 35% or less, 40% or less relative to a control (e.g., phosphate buffered saline) in an assay described herein or known in the art.

In another embodiment, an EphA2/EphrinA1 Modulator is an agent that binds to EphrinA1 and prevents or reduces EphrinA1 signal transduction but does not inhibit or reduce the interaction between EphrinA1 and EphA2. In a particular embodiment, the EphA2/EphrinA1 Modulator reduces EphrinA1 signal transduction by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline) in an assay described herein or known in the art (e.g., an immunoassay). In accordance with this embodiment, the EphA2/EphrinA1 Modulator does not reduce or only reduces the interaction between EphA2 and an endogenous ligand(s) of EphA2 (preferably, EphrinA1) by 5% or less, 10% or less, 15% or less, 20% or less, 25% or less, 30% or less, 35% or less, 40% or less, or 2 fold or less, 1.5 fold or less or 1 fold or less relative to a control (e.g., phosphate buffered saline) in an assay described herein or known in the art.

In a specific embodiment, an EphA2/EphrinA1 Modulator is an EphA2/EphrinA1 Interaction Inhibitor. In one embodiment, an EphA2/EphrinA1 Interaction Inhibitor is an agent that binds to EphA2, prevents or reduces the interaction between EphA2 and an endogenous ligand of EphA2, preferably, EphrinA1, and induces EphA2 signal transduction (e.g., soluble forms of EphrinA1 and antibodies that bind to EphA2, induce signaling and phosphorylation of EphA2 (i.e., an agonistic antibody)). In a particular embodiment, such an EphA2/EphrinA1 Interaction Inhibitor reduces the interaction between EphA2 and an endogenous ligand of EphA2 (preferably, EphrinA1) by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline) in an assay described herein or known in the art. In accordance with this embodiment, the EphA2/EphrinA1 Interaction Inhibitor induces EphA2 signal transduction by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline) in an assay described herein or known in the art (e.g., an immunoassay).

In another embodiment, an EphA2/EphrinA1 Interaction Inhibitor is an agent that binds to EphA2, prevents or reduces the interaction between EphA2 and an endogenous ligand of EphA2, preferably, EphrinA1, and prevents or induces very low to negligible levels of EphA2 signal transduction (e.g., antibodies). In a particular embodiment, such an EphA2/EphrinA1 Interaction Inhibitor reduces the interaction between EphA2 and an endogenous ligand of EphA2 (preferably, EphrinA1) by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline) in an assay described herein or known in the art. In accordance with this embodiment, the EphA2/EphrinA1 Interaction Inhibitor induces EphA2 signal transduction by 40% or less, 35% or less, 30% or less, 25% or less, 20% or less, 15% or less, 10% or less, 5% or less, or 2 fold or less, 1.5 fold or less, or 1 fold or less relative to a control (e.g., phosphate buffered saline) in an assay described herein or known in the art (e.g., an immunoassay).

In another embodiment, an EphA2/EphrinA1 Interaction Inhibitor is an agent that binds to EphrinA1, prevents or reduces the interaction between EphA2 and EphrinA1 and induces EphrinA1 signal transduction (e.g., soluble forms of EphA2, dominant negative forms of EphA2, and antibodies that bind to EphrinA1 and induce EphrinA1 signal transduction). In a particular embodiment, such an EphA2/EphrinA1 Interaction Inhibitor reduces the interaction between EphA2 and EphrinA1 by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline) in an assay described herein or known in the art. In accordance with this embodiment, the EphA2/EphrinA1 Interaction Inhibitor induces EphrinA1 signal transduction by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline) in an assay described herein or known in the art (e.g., an immunoassay).

In another embodiment, an EphA2/EphrinA1 Interaction Inhibitor is an agent that binds to EphrinA1, prevents or reduces the interaction between EphA2 and EphrinA1, and prevents or induces very low to negligible levels of EphrinA1 signal transduction (e.g., antibodies). In a particular embodiment, such an EphA2/EphrinA1 Interaction Inhibitor reduces the interaction between EphA2 and EphrinA1 by at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or at least 1.5 fold, at least 2 fold, at least 2.5 fold, at least 3 fold, at least 3.5 fold, at least 4 fold, at least 4.5, at least 5 fold, at least 7 fold or at least 10 fold relative to a control (e.g., phosphate buffered saline) in an assay described herein or known in the art. In accordance with this embodiment, the EphA2/EphrinA1 Interaction Inhibitor induces EphrinA1 signal transduction by 40% or less, 35% or less, 30% or less, 25% or less, 20% or less, 15% or less, 10% or less, 5% or less, or 2 fold or less, 1.5 fold or less or 1 fold or less relative to a control (e.g., phosphate buffered saline) in an assay described herein or known in the art (e.g., an immunoassay).

In a specific embodiment, an EphA2/EphrinA1 Modulator is not an agent that inhibits or reduces EphA2 gene expression (e.g., EphA2 antisense, RNAi or ribozyme). In another embodiment, an EphA2/EphrinA1 Modulator is not an EphA2/EphrinA1 Inhibitor that is an agent that binds to EphA2, prevents or reduces the interaction between EphA2 and an endogenous ligand of EphA2, preferably, EphrinA1, and induces EphA2 signal transduction. In another embodiment, an antibody that immunospecifically binds to EphA2 and induces signaling and phosphorylation of EphA2 (i.e., an agonistic antibody).

In a specific embodiment, an EphA2/EphrinA1 Modulator has one, two or all of the following cellular effects: (i) increases in the proliferation of EphA2-expressing cells; (ii) increases in the survival of EphA2 expressing cells (by, e.g., a preventing or reducing apoptosis and/or necrosis); and (iii) maintains and/or reconstitutes of the integrity of an epithelial and/or endothelial cell layer. In a particular embodiment, an EphA2/EphrinA1 Modulator prevents, reduces or slows the deposition of extracellular matrix (ECM) components (e.g., collagen, proteoglycans, tenascin and fibronectin). In a specific embodiment, an EphA2/EphrinA1 Modulator of the invention modulates angiogenesis. In a particular embodiment of the invention, an EphA2/EphrinA1 Modulator prevents, reduces or slows down angiogenesis. In an alternative embodiment, an EphA2/EphrinA1 Modulator of the invention increases angiogenesis.

EphA2/EphrinA1 Modulators of the invention include, but are not limited to, proteinaceous molecules (including, but not limited to, peptides, polypeptides, proteins, post-translationally modified proteins, antibodies, Listeria-based and non-Listeria-based vaccines, etc.), small molecules (less than 1000 daltons), inorganic or organic compounds, nucleic acid molecules (including, but not limited to, double-stranded, single-stranded DNA, double-stranded or single-stranded RNA (e.g., antisense, mediates RNAi, etc.), and triple helix nucleic acid molecules), aptamers, and derivatives of any of the above.

4.2 Polypeptides As EphA2/EphrinA1 Modulators

Methods of the present invention encompass EphA2/EphrinA1 Modulators that are polypeptides. In specific embodiment, a polypeptide EphA2/EphrinA1 Modulator prevents, reduces or slows the deposition of ECM components (e.g., collagen) in an epithelial and/or endothelial cell layer. In another specific embodiment, a polypeptide EphA2/EphrinA1 Modulator modulates angiogenesis. In a particular embodiment, a polypeptide EphA2/EphrinA1 Modulator prevents, reduces or slows down angiogenesis. In another embodiment, a polypeptide EphA2/EphrinA1 Modulator increases angiogenesis.

In one embodiment, a polypeptide EphA2/EphrinA1 Modulator is an antibody. In a preferred embodiment, the EphA2/EphrinA1 Modulator antibody is a monoclonal antibody, and more preferably, is human or humanized. In another embodiment, a polypeptide EphA2/EphrinA1 Modulator is a soluble form of EphA2 or EphrinA1. In another embodiment, a polypeptide EphA2/EphrinA1 Modulator is a dominant negative form of EphA2 or EphrinA1.

In one embodiment, a polypeptide EphA2/EphrinA1 Modulator is an EphA2/EphrinA1 Interaction Inhibitor. In a specific embodiment, an EphA2/EphrinA1 Modulator is an EphA2 antibody that immunospecifically binds EphA2, prevents or reduces the interaction between EphA2 and an endogenous ligand of EphA2, preferably, EphrinA1, and induces EphA2 signal transduction (including, but not limited to, EphA2 autophosphorylation). In another embodiment, an EphA2/EphrinA1 Modulator is an EphA2 antibody that immunospecifically binds to EphA2, prevents or reduces the interaction between EphA2 and an endogenous ligand of EphA2, preferably, EphrinA1, and prevents or induces very low to negligible levels of EphA2 signal transduction (including, but not limited to, autophosphorylation of EphA2). In certain embodiments, a polypeptide EphA2/EphrinA1 Modulator is not an EphA2 antibody that immunospecifically binds to EphA2, prevents or reduces the interaction between EphA2 and EphrinA1, and induces EphA2 signal transduction.

In a specific embodiment, a polypeptide EphA2/EphrinA1 Modulator is an EphrinA1 antibody that immunospecifically binds to EphrinA1, prevents or reduces the interaction between EphA1 and EphrinA1, and induces EphrinA1 signal transduction. In another embodiment, an EphA2/EphrinA1 Modulator is an EphrinA1 antibody that immunospecifically binds EphrinA1, prevents or reduces the interaction between EphA2 and EphrinA1, and prevents or induces very low to negligible levels of EphrinA1 signal transduction.

In a specific embodiment, an EphA2/EphrinA1 Modulator is a soluble form of EphrinA1 or a fragment of EphrinA1 that binds EphA2, prevents or reduces the interaction between EphA2 and EphrinA1, and induces EphA2 signal transduction (including, but not limited to, autophosphorylation). In another embodiment, an EphA2/EphrinA1 Modulator is a soluble form of EphrinA1 or a fragment of EphrinA1 that binds to EphA2, prevents or reduces the interaction between EphA2 and EphrinA1, and prevents or induces very low to negligible levels of EphA2 signal transduction (including, but not limited to, autophosphorylation of EphA2).

In a specific embodiment, an EphA2/EphrinA1 Modulator is a soluble form of EphA2 or a fragment of EphA2 that binds to an endogenous ligand of EphA2 (preferably, EphrinA1), prevents or reduces the interaction between EphA2 and an endogenous ligand of EphA2 (preferably, EphrinA1), and induces EphrinA1 signal transduction. In another embodiment, an EphA2/EphrinA1 Modulator is a soluble form of EphA2 or a fragment of EphA2 that binds to an endogenous ligand of EphA2 (preferably, EphrinA1), prevents or reduces the interaction between EphA2 and an endogenous ligand of EphA2 (preferably, EphrinA1), and prevents or induces very low to negligible levels of EphrinA1 signal transduction.

In a specific embodiment, an EphA2/EphrinA1 Modulator is a dominant negative form of EphA2 that binds to an endogenous ligand of EphA2 (preferably, EphrinA1), prevents or reduces the interaction between EphA2 and an endogenous ligand of EphA2 (preferably, EphrinA1), and induces EphrinA1 signal transduction. In another embodiment, an EphA2/EphrinA1 Modulator is a dominant negative form of EphA2 that binds to an endogenous ligand of EphA2 (preferably, EphrinA1), prevents or reduces the interaction between EphA2 and an endogenous ligand of EphA2 (preferably, EphrinA1), and prevents or induces very low to negligible levels of EphrinA1 signal transduction.

The present invention encompass the proteinaceous EphA2/EphrinA1 Modulators (e.g., antibody and polypeptide EphA2/EphrinA1 Modulators) that have half-lives (e.g., serum half-lives) in a mammal, preferably a human, of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the proteinaceous EphA2/EphrinA1 Modulators in mammals, preferably humans, results in a higher concentration of said proteinaceous EphA2/EphrinA1 Modulators in the mammals, and thus, reduces the frequency of the administration of said polypeptide EphA2/EphrinA1 Modulators and/or reduces the amount of said proteinaceous EphA2/EphrinA1 Modulators to be administered. Proteinaceous EphA2/EphrinA1 Modulators having increased in vivo half-lives can be generated by techniques known to those of skill in the art. For example, proteinaceous EphA2/EphrinA1 Modulators with increased in vivo half-lives can be generated by modifying (e.g., substituting, deleting or adding) amino acid residues. In one embodiment, when the proteinaceous EphA2/EphrinA1 Modulator is an antibody, such amino acid residues to be modified can be those residues involved in the interaction between the Fc domain and the FcRn receptor (see, e.g., International Patent Publication No. WO 97/34631 and U.S. patent application Ser. No. 10/020,354 filed Dec. 12, 2001 entitled “Molecules With Extended Half-Lives, Compositions and Uses Thereof,” which are incorporated herein by reference in their entireties). Proteinaceous EphA2/EphrinA1 Modulators with increased in vivo half-lives can also be generated by attaching to said polypeptides polymer molecules such as high molecular weight polyethylene glycol (PEG). PEG can be attached to said proteinaceous EphA2/EphrinA1 Modulators with or without a multifunctional linker either through site-specific conjugation of the PEG to the N- or C-terminus of said polypeptide or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the proteinaceous EphA2/EphrinA1 Modulators. Unreacted PEG can be separated from proteinaceous EphA2/EphrinA1 Modulator-PEG conjugates by, e.g., size exclusion or ion-exchange chromatography.

4.2.1 Antibodies as EphA2/EphrinA1 Modulators

In one embodiment, an EphA2/EphrinA1 Modulator is an antibody, preferably a monoclonal antibody. In another preferred embodiment, the antibody is human or humanized. Antibody EphA2/EphrinA1 Modulators of the invention immunospecifically bind EphA2 or EphrinA1 and modulate the activity and/or expression of EphA2 and/or EphrinA1. In a specific embodiment, the antibody prevents, reduces or slows the deposition of ECM components (e.g., collagen) in an epithelial and/or endothelial cell layer. In another specific embodiment, the antibody modulates angiogenesis. In a particular embodiment, the antibody prevents, reduces or slows down angiogenesis. In an alternative embodiment, the antibody increases angiogenesis.

In a specific embodiment, an antibody of the invention immunospecifically binds to the extracellular domain of EphA2 (e.g., at an epitope either within or outside of the EphA2 ligand binding site) and decreases EphA2 cytoplasmic tail phosphorylation without causing EphA2 degradation. In another specific embodiment, the antibody binds to the extracellular domain of EphA2 (e.g., at an epitope either within or outside of the EphA2 ligand binding site) and inhibits or reduces the extent of EphA2-ligand interaction. In another specific embodiment, an antibody of the invention immunospecifically binds to the extracellular domain of EphA2 (e.g., at an epitope either within or outside of the EphA2 ligand binding site) and decreases EphA2 signal transduction (including, but not limited to, EphA2 autophosphorylation). In yet another embodiment, an antibody of the invention immunospecifically binds to the extracellular domain of EphA2 (e.g., at an epitope either within or outside of the EphA2 ligand binding site), decreases EphA2 signal transduction (including, but not limited to, EphA2 autophosphorylation) and inhibits or reduces the extent of EphA2-ligand interaction. In a specific embodiment, an antibody of the invention immunospecifically binds to the ligand binding domain of human EphA2 (e.g., at amino acid residues 28 to 201) as disclosed in the GenBank database (GenBank accession no. NP_{—}004422.2).

In one embodiment, an antibody of the invention immunospecifically binds to EphrinA1 (e.g., at an epitope either within or outside of the EphA2 binding site) and prevents or reduces the binding to EphA2. In another embodiment, the EphrinA1 antibody of the invention immunospecifically binds to EphrinA1 (e.g., at an epitope either within or outside of the EphA2 binding site) and modulates (induces or inhibits) EphrinA1 signaling in an EphrinA1 expressing cell. In another specific embodiment, an antibody of the invention immunospecifically binds to EphrinA1 (e.g., at an epitope either within or outside of the EphA2 binding site), decreases EphrinA1 signal transduction and inhibits or reduces the extent of EphA2-EphrinA1 interaction. In another specific embodiment, an antibody of the invention immunospecifically binds to EphrinA1 (e.g., at an epitope either within or outside of the EphA2 binding site), induces EphrinA1 signal transduction and inhibits or reduces the extent of EphA2-EphrinA1 interaction. In a further embodiment, an antibody of the invention immunospecifically binds to EphrinA1 (e.g., at an epitope involved in EphrinA1 clustering), inhibits or reduces EphrinA1 interaction with other molecules such as the Src family kinases (e.g., Fyn), and inhibits or reduces EphrinA1 signal transduction.

Antibodies of the invention include, but are not limited to, synthetic antibodies, monoclonal antibodies, recombinantly produced antibodies, multispecific antibodies (including bi-specific antibodies), human antibodies, humanized antibodies, chimeric antibodies, intrabodies, single-chain Fvs (scFv) (e.g., including monospecific and bi-specific, etc.), Fab fragments, F(ab′) fragments, disulfide-linked Fvs (sdFv), anti-idiotypic (anti-Id) antibodies, and epitope-binding fragments of any of the above. In particular, antibodies of the present invention include immunoglobulin molecules and immunologically active portions of immunoglobulin molecules, i.e., molecules that contain an antigen-binding site that immunospecifically binds to an EphA2 antigen or an EphrinA1 antigen (e.g., one or more complementarity determining regions (CDRs) of an anti-EphA2 antibody or of an anti-EphrinA1 antibody). The antibodies of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

The present invention encompasses agonistic antibodies that immunospecifically bind to EphA2 and agonize EphA2, i.e., elicit EphA2 signaling and decrease EphA2 expression. Agonistic EphA2 antibodies may induce EphA2 autophosphorylation, thereby causing subsequent EphA2 degradation to down-regulate EphA2 expression and inhibit EphA2 interaction with its endogenous ligand (e.g., EphrinA1). Such antibodies are disclosed in U.S. Patent Pub. Nos. US 2004/0091486 A1 (May 13, 2004), and US 2004/0028685 A1 (Feb. 12, 2004), which are incorporated by reference herein in there entireties.

The present invention also encompasses single domain antibodies, including camelized single domain antibodies (see, e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Patent Publication Nos. WO 94/04678 and WO 94/25591; U.S. Pat. No. 6,005,079; which are incorporated herein by reference in their entireties). In one embodiment, the present invention provides single domain antibodies comprising two V_{H} domains having the amino acid sequence of a V_{H} domain(s) of any EphA2 or EphrinA1 antibody(ies) with modifications such that single domain antibodies are formed. In another embodiment, the present invention also provides single domain antibodies comprising two V_{H} domains comprising one or more of the V_{H} CDRs of any EphA2 or EphrinA1 antibody(ies).

Antibodies of the invention include EphA2 or EphrinA1 intrabodies (see Section 4.2.1.1). Antibody EphA2/EphrinA1 Modulators of the invention that are intrabodies immunospecifically bind EphA2 or EphrinA1 and modulate (increase or decrease) the expression and/or activity of EphA2 or EphrinA1. In a specific embodiment, an intrabody of the invention immunospecifically binds to the intracellular domain of EphA2 and decreases EphA2 cytoplasmic tail phosphorylation without causing EphA2 degradation. In another embodiment, an intrabody of the invention immunospecifically binds to EphA2 and prevents or reduces EphA2 signal transduction (including, but not limited to EphA2 autophosphorylation) but does not inhibit or reduce the interaction between EphA2 and an endogenous ligand(s) of EphA2, preferably, EphrinA1.

The antibodies used in the methods of the invention may be from any animal origin including birds and mammals (e.g., human, murine, donkey, sheep, rabbit, goat, guinea pig, camel, horse, or chicken). In a most preferred embodiment, the antibody is human or has been humanized. As used herein, “human” antibodies include antibodies having the amino acid sequence of a human immunoglobulin and include antibodies isolated from human immunoglobulin libraries or from mice that express antibodies from human genes.

The antibodies used in the methods of the present invention may be monospecific, bispecific, trispecific or of greater multispecificity. Multispecific antibodies may immunospecifically bind to different epitopes of an EphA2 polypeptide or an EphrinA1 polypeptide or may immunospecifically bind to both an EphA2 polypeptide or an EphrinA1 polypeptide as well a heterologous epitope, such as a heterologous polypeptide or solid support material. See, e.g., International Patent Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553.

4.2.1.1 Intrabodies

In certain embodiments, the antibody to be used with the invention binds to an intracellular epitope, i.e., is an intrabody. In a specific embodiment, an intrabody of the invention binds to the cytoplasmic domain of EphA2 and prevents EphA2 signaling (e.g., autophosphorylation). An intrabody comprises at least a portion of an antibody that is capable of immunospecifically binding an antigen and preferably does not contain sequences coding for its secretion. Such antibodies will bind antigen intracellularly. In one embodiment, the intrabody comprises a single-chain Fv (“scFv”). scFvs are antibody fragments comprising the V_{H} and V_{L} domains of antibody, wherein these domains are present in a single polypeptide chain. Generally, the scFv polypeptide further comprises a polypeptide linker between the V_{H} and V_{L} domains which enables the scFv to form the desired structure for antigen binding. For a review of scFvs see Pluckthun in The Pharmacology of Monoclonal Antibodies, vol. 113, Rosenburg and Moore eds. Springer-Verlag, New York, pp. 269-315 (1994). In a further embodiment, the intrabody preferably does not encode an operable secretory sequence and thus remains within the cell (see generally Marasco, Wash., 1998, “Intrabodies: Basic Research and Clinical Gene Therapy Applications” Springer:New York).

Generation of intrabodies is well-known to the skilled artisan and is described, for example, in U.S. Pat. Nos. 6,004,940; 6,072,036; 5,965,371, which are incorporated by reference in their entireties herein. Further, the construction of intrabodies is discussed in Ohage and Steipe, 1999, J. Mol. Biol. 291:1119-1128; Ohage et al., 1999, J. Mol. Biol. 291:1129-1134; and Wirtz and Steipe, 1999, Protein Science 8:2245-2250, which references are incorporated herein by reference in their entireties. Recombinant molecular biological techniques such as those described for recombinant production of antibodies may also be used in the generation of intrabodies.

In one embodiment, intrabodies of the invention retain at least about 75% of the binding effectiveness of the complete antibody (i.e., having the entire constant domain as well as the variable regions) to the antigen. More preferably, the intrabody retains at least 85% of the binding effectiveness of the complete antibody. Still more preferably, the intrabody retains at least 90% of the binding effectiveness of the complete antibody. Even more preferably, the intrabody retains at least 95% of the binding effectiveness of the complete antibody.

In producing intrabodies, polynucleotides encoding variable region for both the V_{H} and V_{L} chains of interest can be cloned by using, for example, hybridoma mRNA or splenic mRNA as a template for PCR amplification of such domains (Huse et al., 1989, Science 246:1276). In one preferred embodiment, the polynucleotides encoding the V_{H} and V_{L} domains are joined by a polynucleotide sequence encoding a linker to make a single chain antibody (scFv). The scFv typically comprises a single peptide with the sequence V_{H}-linker-V_{L} or V_{L}-linker-V_{H}. The linker is chosen to permit the heavy chain and light chain to bind together in their proper conformational orientation (see for example, Huston et al., 1991, Methods in Enzym. 203:46-121, which is incorporated herein by reference). In a further embodiment, the linker can span the distance between its points of fusion to each of the variable domains (e.g., 3.5 nm) to minimize distortion of the native Fv conformation. In such an embodiment, the linker is a polypeptide of at least 5 amino acid residues, at least 10 amino acid residues, at least 15 amino acid residues, or greater. In a further embodiment, the linker should not cause a steric interference with the V_{H} and V_{L} domains of the combining site. In such an embodiment, the linker is 35 amino acids or less, 30 amino acids or less, or 25 amino acids or less. Thus, in a most preferred embodiment, the linker is between 15-25 amino acid residues in length. In a further embodiment, the linker is hydrophilic and sufficiently flexible such that the V_{H} and V_{L} domains can adopt the conformation necessary to detect antigen. Intrabodies can be generated with different linker sequences inserted between identical V_{H} and V_{L} domains. A linker with the appropriate properties for a particular pair of V_{H} and V_{L} domains can be determined empirically by assessing the degree of antigen binding for each. Examples of linkers include, but are not limited to, those sequences disclosed in Table 3.

TABLE 3

| | |
|---|---|
| Sequence | SEQ ID NO. |

| | |
|---|---|
| (Gly Gly Gly Gly Ser)₃ | SEQ ID NO:1 |
| Glu Ser Gly Arg Ser Gly Gly Gly Gly Ser | SEQ ID NO:2 |
| Gly Gly Gly Gly Ser | |
| Glu Gly Lys Ser Ser Gly Ser Gly Ser Glu | SEQ ID NO:3 |
| Ser Lys Ser Thr | |
| Glu Gly Lys Ser Ser Gly Ser Gly Ser Glu | SEQ ID NO:4 |
| Ser Lys Ser Thr Gln | |
| Glu Gly Lys Ser Ser Gly Ser Gly Ser Glu | SEQ ID NO:5 |
| Ser Lys Val Asp | |
| Gly Ser Thr Ser Gly Ser Gly Lys Ser Ser | SEQ ID NO:6 |
| Glu Gly Lys Gly | |
| Lys Glu Ser Gly Ser Val Ser Ser Glu Glu | SEQ ID NO:7 |
| Leu Ala Gln Phe Arg Ser Leu Asp | |
| Glu Ser Gly Ser Val Ser Ser Glu Glu Leu | SEQ ID NO:8 |
| Ala Phe Arg Ser Leu Asp | |

In one embodiment, intrabodies are expressed in the cytoplasm. In other embodiments, the intrabodies are localized to various intracellular locations. In such embodiments, specific localization sequences can be attached to the intrabody polypeptide to direct the intrabody to a specific location. Intrabodies can be localized, for example, to the following intracellular locations: endoplasmic reticulum (Munro et al., 1987, Cell 48:899-907; Hangejorden et al., 1991, J. Biol. Chem. 266:6015); nucleus (Lanford et al., 1986, Cell 46:575; Stanton et al., 1986, PNAS 83:1772; Harlow et al., 1985, Mol. Cell Biol. 5:1605; Pap et al., 2002, Exp. Cell Res. 265:288-93); nucleolar region (Seomi et al., 1990, J. Virology 64:1803; Kubota et al., 1989, Biochem. Biophys. Res. Comm. 162:963; Siomi et al., 1998, Cell 55:197); endosomal compartment (Bakke et al., 1990, Cell 63:707-716); mitochondrial matrix (Pugsley, A. P., 1989, “Protein Targeting”, Academic Press, Inc.); Golgi apparatus (Tang et al., 1992, J. Bio. Chem. 267:10122-6); liposomes (Letoumeur et al., 1992, Cell 69:1183); peroxisome (Pap et al., 2002, Exp. Cell Res. 265:288-93); trans Golgi network (Pap et al., 2002, Exp. Cell Res. 265:288-93); and plasma membrane (Marchildon et al., 1984, PNAS 81:7679-82; Henderson et al., 1987, PNAS 89:339-43; Rhee et al., 1987, J. Virol. 61:1045-53; Schultz et al., 1984, J. Virol. 133:431-7; Ootsuyama et al., 1985, Jpn. J. Can. Res. 76:1132-5; Ratner et al., 1985, Nature 313:277-84). Examples of localization signals include, but are not limited to, those sequences disclosed in Table 4.

TABLE 4

| | | |
|---|---|---|
| Localization | Sequence | SEQ ID NO. |

| | | |
|---|---|---|
| endoplasmic reticulum | Lys Asp Glu Leu | SEQ ID NO:9 |
| endoplasmic reticulum | Asp Asp Glu Leu | SEQ ID NO:10 |
| endoplasmic reticulum | Asp Glu Glu Leu | SEQ ID NO:11 |
| endoplasmic reticulum | Gln Glu Asp Leu | SEQ ID NO:12 |
| endoplasmic reticulum | Arg Asp Glu Leu | SEQ ID NO:13 |
| Nucleus | Pro Lys Lys Lys Arg Lys Val | SEQ ID NO:14 |
| Nucleus | Pro Gln Lys Lys Ile Lys Ser | SEQ ID NO:15 |
| Nucleus | Gln Pro Lys Lys Pro | SEQ ID NO:16 |
| Nucleus | Arg Lys Lys Arg | SEQ ID NO:17 |
| Nucleus | Lys Lys Lys Arg Lys | SEQ ID NO:18 |
| nucleolar region | Arg Lys Lys Arg Arg Gln Arg Arg Arg Ala | SEQ ID NO:19 |
| | His Gln | |
| nucleolar region | Arg Gln Ala Arg Arg Asn Arg Arg Arg Arg | SEQ ID NO:20 |
| | Trp Arg Glu Arg Gln Arg | |
| nucleolar region | Met Pro Leu Thr Arg Arg Arg Pro Ala Ala | SEQ ID NO:21 |
| | Ser Gln Ala Leu Ala Pro Pro Thr Pro | |
| endosomal compartment | Met Asp Asp Gln Arg Asp Leu Ile Ser Asn | SEQ ID NO:22 |
| | Asn Glu Gln Leu Pro | |
| mitochondrial matrix | Met Leu Phe Asn Leu Arg Xaa Xaa Leu Asn | SEQ ID NO:23 |
| | Asn Ala Ala Phe Arg His Gly His Asn Phe | |
| | Met Val Arg Asn Phe Arg Cys Gly Gln Pro | |
| | Leu Xaa | |
| Peroxisome | Ala Lys Leu | SEQ ID NO:24 |
| trans Golgi network | Ser Asp Tyr Gln Arg Leu | SEQ ID NO:25 |
| plasma membrane | Gly Cys Val Cys Ser Ser Asn Pro | SEQ ID NO:26 |
| plasma membrane | Gly Gln Thr Val Thr Thr Pro Leu | SEQ ID NO:27 |
| plasma membrane | Gly Gln Glu Leu Ser Gln His Glu | SEQ ID NO:28 |
| plasma membrane | Gly Asn Ser Pro Ser Tyr Asn Pro | SEQ ID NO:29 |
| plasma membrane | Gly Val Ser Gly Ser Lys Gly Gln | SEQ ID NO:30 |
| plasma membrane | Gly Gln Thr Ile Thr Thr Pro Leu | SEQ ID NO:31 |
| plasma membrane | Gly Gln Thr Leu Thr Thr Pro Leu | SEQ ID NO:32 |
| plasma membrane | Gly Gln Ile Phe Ser Arg Ser Ala | SEQ ID NO:33 |
| plasma membrane | Gly Gln Ile His Gly Leu Ser Pro | SEQ ID NO:34 |
| plasma membrane | Gly Ala Arg Ala Ser Val Leu Ser | SEQ ID NO:35 |
| plasma membrane | Gly Cys Thr Leu Ser Ala Glu Glu | SEQ ID NO:36 |

V_{H} and V_{L} domains are made up of the immunoglobulin domains that generally have a conserved structural disulfide bond. In embodiments where the intrabodies are expressed in a reducing environment (e.g., the cytoplasm), such a structural feature cannot exist. Mutations can be made to the intrabody polypeptide sequence to compensate for the decreased stability of the immunoglobulin structure resulting from the absence of disulfide bond formation. In one embodiment, the V_{H} and/or V_{L} domains of the intrabodies contain one or more point mutations such that their expression is stabilized in reducing environments (see Steipe et al., 1994, J. Mol. Biol. 240:188-92; Wirtz and Steipe, 1999, Protein Science 8:2245-50; Ohage and Steipe, 1999, J. Mol. Biol. 291:1119-28; Ohage et al., 1999, J. Mol. Biol. 291:1129-34).

Intrabody Proteins as Therapeutics

In one embodiment, the recombinantly expressed intrabody protein is administered to a patient. Such an intrabody polypeptide must be intracellular to mediate a prophylactic or therapeutic effect. In this embodiment of the invention, the intrabody polypeptide is associated with a “membrane permeable sequence”. Membrane permeable sequences are polypeptides capable of penetrating through the cell membrane from outside of the cell to the interior of the cell. When linked to another polypeptide, membrane permeable sequences can also direct the translocation of that polypeptide across the cell membrane as well.

In one embodiment, the membrane permeable sequence is the hydrophobic region of a signal peptide (see, e.g., Hawiger, 1999, Curr. Opin. Chem. Biol. 3:89-94; Hawiger, 1997, Curr. Opin. Immunol. 9:189-94; U.S. Pat. Nos. 5,807,746 and 6,043,339, which are incorporated herein by reference in their entireties). The sequence of a membrane permeable sequence can be based on the hydrophobic region of any signal peptide. The signal peptides can be selected, e.g., from the SIGPEP database (see e.g., von Heijne, 1987, Prot. Seq. Data Anal. 1:41-2; von Heijne and Abrahmsen, 1989, FEBS Lett. 224:439-46). When a specific cell type is to be targeted for insertion of an intrabody polypeptide, the membrane permeable sequence is preferably based on a signal peptide endogenous to that cell type. In another embodiment, the membrane permeable sequence is a viral protein (e.g., Herpes Virus Protein VP22) or fragment thereof (see e.g., Phelan et al., 1998, Nat. Biotechnol. 16:440-3). A membrane permeable sequence with the appropriate properties for a particular intrabody and/or a particular target cell type can be determined empirically by assessing the ability of each membrane permeable sequence to direct the translocation of the intrabody across the cell membrane. Examples of membrane permeable sequences include, but are not limited to, those sequences disclosed in Table 5, infra.

TABLE 5

| | |
|---|---|
| Sequence | SEQ ID NO. |

| | |
|---|---|
| Ala Ala Val Ala Leu Leu Pro Ala Val | SEQ ID NO:37 |
| Leu Leu Ala Leu Leu Ala Pro | |
| Ala Ala Val Leu Leu Pro Val Leu Leu | SEQ ID NO:38 |
| Ala Ala Pro | |
| Val Thr Val Leu Ala Leu Gly Ala Leu | SEQ ID NO:39 |
| Ala Gly Val Gly Val Gly | |

In another embodiment, the membrane permeable sequence can be a derivative. In this embodiment, the amino acid sequence of a membrane permeable sequence has been altered by the introduction of amino acid residue substitutions, deletions, additions, and/or modifications. For example, but not by way of limitation, a polypeptide may be modified, e.g., by glycosylation, acetylation, pegylation, phosphorylation, amidation, derivatization by known protecting/blocking groups, proteolytic cleavage, linkage to a cellular ligand or other protein, etc. A derivative of a membrane permeable sequence polypeptide may be modified by chemical modifications using techniques known to those of skill in the art, including, but not limited to specific chemical cleavage, acetylation, formylation, metabolic synthesis of tunicamycin, etc. Further, a derivative of a membrane permeable sequence polypeptide may contain one or more non-classical amino acids. In one embodiment, a polypeptide derivative possesses a similar or identical function as an unaltered polypeptide. In another embodiment, a derivative of a membrane permeable sequence polypeptide has an altered activity when compared to an unaltered polypeptide. For example, a derivative membrane permeable sequence polypeptide can translocate through the cell membrane more efficiently or be more resistant to proteolysis.

The membrane permeable sequence can be attached to the intrabody in a number of ways. In one embodiment, the membrane permeable sequence and the intrabody are expressed as a fusion protein. In this embodiment, the nucleic acid encoding the membrane permeable sequence is attached to the nucleic acid encoding the intrabody using standard recombinant DNA techniques (see e.g., Rojas et al., 1998, Nat. Biotechnol. 16:370-5). In a further embodiment, there is a nucleic acid sequence encoding a spacer peptide placed in between the nucleic acids encoding the membrane permeable sequence and the intrabody. In another embodiment, the membrane permeable sequence polypeptide is attached to the intrabody polypeptide after each is separately expressed recombinantly (see e.g., Zhang et al., 1998, PNAS 95:9184-9). In this embodiment, the polypeptides can be linked by a peptide bond or a non-peptide bond (e.g. with a crosslinking reagent such as glutaraldehyde or a thiazolidino linkage see e.g., Hawiger, 1999, Curr. Opin. Chem. Biol. 3:89-94) by methods standard in the art.

The administration of the membrane permeable sequence-intrabody polypeptide can be by parenteral administration, e.g., by intravenous injection including regional perfusion through a blood vessel supplying the tissues(s) or organ(s) having the target cell(s), or by inhalation of an aerosol, subcutaneous or intramuscular injection, topical administration such as to skin wounds and lesions, direct transfection into, e.g., bone marrow cells prepared for transplantation and subsequent transplantation into the subject, and direct transfection into an organ that is subsequently transplanted into the subject. Further administration methods include oral administration, particularly when the complex is encapsulated, or rectal administration, particularly when the complex is in suppository form. A pharmaceutically acceptable carrier includes any material that is not biologically or otherwise undesirable, i.e., the material may be administered to an individual along with the selected complex without causing any undesirable biological effects or interacting in a deleterious manner with any of the other components of the pharmaceutical composition in which it is contained.

Conditions for the administration of the membrane permeable sequence-intrabody polypeptide can be readily be determined, given the teachings in the art (see e.g., Remington's Pharmaceutical Sciences, 18^{th} Ed., E. W. Martin (ed.), Mack Publishing Co., Easton, Pa. (1990)). If a particular cell type in vivo is to be targeted, for example, by regional perfusion of an organ or tumor, cells from the target tissue can be biopsied and optimal dosages for import of the complex into that tissue can be determined in vitro to optimize the in vivo dosage, including concentration and time length. Alternatively, culture cells of the same cell type can also be used to optimize the dosage for the target cells in vivo.

Intrabody Gene Therapy as Therapeutic

In another embodiment, a polynucleotide encoding an intrabody is administered to a patient (e.g., as in gene therapy). In this embodiment, methods as described in Section 4.8.1 can be used to administer the polynucleotide of the invention.

4.2.1.2 Antibody Conjugates

The present invention encompasses the use of EphA2/EphrinA1 Modulators (e.g., EphA2 and/or EphrinA1 antibodies or fragments thereof that immunospecifically bind to EphA2 and/or EphrinA1) that are recombinantly fused or chemically conjugated (including both covalent and non-covalent conjugations) to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. For example, antibodies may be used to target heterologous polypeptides to particular cell types, either in vitro or in vivo, by fusing or conjugating the antibodies to antibodies specific for particular cell surface receptors. Antibodies fused or conjugated to heterologous polypeptides may also be used in in vitro immunoassays and purification methods using methods known in the art. See e.g., International Publication WO 93/21232; EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452, which are incorporated by reference in their entireties. In specific embodiments, the disorder to be detected, treated, managed, or monitored is a non-neoplastic hyperproliferative epithelial and/or endothelial cell disorder, including but not limited to disorders associated with increased deposition of extracellular matrix components (e.g., collagen, proteoglycans, tenascin and fibronectin) and/or aberrant angiogenesis. Non-limiting examples of such disorders include cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis.

The present invention further includes compositions comprising heterologous polypeptides fused or conjugated to antibody fragments. For example, the heterologous polypeptides may be fused or conjugated to a Fab fragment, Fd fragment, Fv fragment, F(ab)₂ fragment, or portion thereof. Methods for fusing or conjugating proteins, polypeptides, or peptides to an antibody or an antibody fragment are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341 (said references are incorporated herein by reference in their entireties).

Additional fusion proteins, e.g., of any of the EphA2 or EphrinA1 Modulators of the invention, may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16:76; Hansson, et al., 1999, J. Mol. Biol. 287:265; and Lorenzo and Blasco, 1998, BioTechniques 24:308 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment, which portions immunospecifically bind to EphA2 or EphrinA1 may be recombined with one or more components, motifs, sections, parts, domains, fragments, etc. of one or more heterologous molecules.

Moreover, the antibodies or fragments thereof can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, PNAS 86:821, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In other embodiments, antibodies of the present invention or fragments or variants thereof are conjugated to a diagnostic or detectable agent. Such antibodies can be useful for monitoring or prognosing the development or progression of a cancer as part of a clinical testing procedure, such as determining the efficacy of a particular therapy. Additionally, such antibodies can be useful for monitoring or prognosing the development or progression of a non-neoplastic hyperproliferative epithelial and/or endothelial cell disorder including but not limited to a disorder associated with increased deposition of extracellular matrix components (e.g., collagen, proteoglycans, tenascin and fibronectin) and/or aberrant angiogenesis. Non-limiting examples of such disorders include cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis. In one embodiment, an EphA2 antibody or an EphrinA1 antibody of the invention is conjugated to a diagnostic or detectable agent. In a more specific embodiment, the antibody is an EphA2 antibody or an EphrinA1 antibody.

Such diagnosis and detection can accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Ph), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

The present invention further encompasses uses of antibodies or fragments thereof conjugated to a prophylactic or therapeutic agent. An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Therapeutic moieties include, but are not limited to, antimetabolites (e.g., methotrexate, 6-mercaptopurine, 6-thioguanine, cytarabine, 5-fluorouracil decarbazine); alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP), and cisplatin); anthracyclines (e.g., daunorubicin (formerly daunomycin) and doxorubicin); antibiotics (e.g., dactinomycin (formerly actinomycin), bleomycin, mithramycin, and anthramycin (AMC)); Auristatin molecules (e.g., auristatin PHE, bryostatin 1, and solastatin 10; see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999), all of which are incorporated herein by reference); hormones (e.g., glucocorticoids, progestins, androgens, and estrogens), DNA-repair enzyme inhibitors (e.g., etoposide or topotecan), kinase inhibitors (e.g., compound ST1571, imatinib mesylate (Kantarjian et al., Clin Cancer Res. 8(7):2167-76 (2002)); cytotoxic agents (e.g., paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucorticoids, procaine, tetracaine, lidocaine, propranolol, and puromycin and analogs or homologs thereof and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459); farnesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305); topoisomerase inhibitors (e.g., camptothecin; irinotecan; SN-38; topotecan; 9-aminocamptothecin; GG-211 (GI 147211); DX-895 If; IST-622; rubitecan; pyrazoloacridine; XR-5000; saintopin; UCE6; UCE1022; TAN-1518A; TAN-1518B; KT6006; KT6528; ED-110; NB-506; ED-110; NB-506; and rebeccamycin); bulgarein; DNA minor groove binders such as Hoescht dye 33342 and Hoechst dye 33258; nitidine; fagaronine; epiberberine; coralyne; beta-lapachone; BC-4-1; bisphosphonates (e.g., alendronate, cimadronte, clodronate, tiludronate, etidronate, ibandronate, neridronate, olpandronate, risedronate, piridronate, pamidronate, zolendronate) HMG-CoA reductase inhibitors, (e.g., lovastatin, simvastatin, atorvastatin, pravastatin, fluvastatin, statin, cerivastatin, lescol, lupitor, rosuvastatin and atorvastatin); antisense oligonucleotides (e.g., those disclosed in the U.S. Pat. Nos. 6,277,832, 5,998,596, 5,885,834, 5,734,033, and 5,618,709); adenosine deaminase inhibitors (e.g., Fludarabine phosphate and 2-Chlorodeoxyadenosine); ibritumomab tiuxetan (Zevalin®); tositumomab (Bexxar®)) and pharmaceutically acceptable salts, solvates, clathrates, and prodrugs thereof. In a specific embodiment, the prophylactic or therapeutic agent to be conjugated to an EphA2/EphrinA1 Modulator of the invention is not cytotoxic to a target cell (e.g., an EphA2- or EphrinA1-expressing cell).

Moreover, an antibody can be conjugated to therapeutic moieties such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the antibody via a linker molecule. Such linker molecules are commonly known in the art and described in Denardo et al., 1998, Clin Cancer Res. 4:2483-90; Peterson et al., 1999, Bioconjug. Chem. 10:553; and Zimmerman et al., 1999, Nucl. Med. Biol. 26:943-50 each incorporated by reference in their entireties.

Further, an antibody or fragment thereof may be conjugated to a prophylactic or therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567-1574), and VEGF (see, International Publication No. WO 99/23105), an anti-angiogenic agent, e.g., angiostatin, endostatin or a component of the coagulation pathway (e.g., tissue factor); or, a biological response modifier such as, for example, a lymphokine (e.g., interferon gamma (“IFN-γ”), interleukin-1 (“IL-1”), interleukin-2 (“IL-2”), interleukin-4 (“IL-4”), interleukin-5 (“IL-5”), interleukin-6 (“IL-6”), interleuking-7 (“IL-7”), interleukin-10 (“IL-10”), interleukin-12 (“IL-12”), interleukin-15 (“IL-15”), interleukin-23 (“IL-23”), granulocyte macrophage colony stimulating factor (“GM-CSF”), and granulocyte colony stimulating factor (“G-CSF”)), or a growth factor (e.g., growth hormone (“GH”)), or a coagulation agent (e.g., calcium, vitamin K, tissue factors, such as but not limited to, Hageman factor (factor XII), high-molecular-weight kininogen (HMWK), prekallikrein (PK), coagulation proteins-factors II (prothrombin), factor V, XIIa, VIII, XIIIa, XI, XIa, IX, IXa, X, phospholipid fibrinopeptides A and B from the α and β chains of fibrinogen, fibrin monomer). In a specific embodiment, an antibody that immunospecifically binds to an IL-9 polypeptide is conjugated with a leukotriene antagonist (e.g., montelukast, zafirlukast, pranlukast, and zyleuton).

Moreover, an antibody can be conjugated to prophylactic or therapeutic moieties such as a radioactive metal ion, such as alpha-emitters such as ²¹³Bi or macrocyclic chelators useful for conjugating radiometal ions, including but not limited to, ¹³¹In, ¹³¹L, ¹³¹Y, ¹³¹Ho, ¹³¹Sm, to polypeptides or any of those listed supra. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

In another embodiment, antibodies can be fused or conjugated to liposomes, wherein the liposomes are used to encapsulate prophylactic or therapeutic agents (see e.g., Park et al., 1997, Can. Lett. 118:153-160; Lopes de Menezes et al., 1998, Can. Res. 58:3320-30; Tseng et al., 1999, Int. J. Can. 80:723-30; Crosasso et al., 1997, J. Pharm. Sci. 86:832-9). In a preferred embodiment, the pharmokinetics and clearance of liposomes are improved by incorporating lipid derivatives of PEG into liposome formulations (see, e.g., Allen et al., 1991, Biochem Biophys Acta 1068:133-41; Huwyler et al., 1997, J. Pharmacol. Exp. Ther. 282:1541-6).

Techniques for conjugating prophylactic or therapeutic moieties to antibodies are well known. Moieties can be conjugated to antibodies by any method known in the art, including, but not limited to aldehyde/Schiff linkage, sulphydryl linkage, acid-labile linkage, cis-aconityl linkage, hydrazone linkage, enzymatically degradable linkage (see generally Garnett, 2002, Adv. Drug Deliv. Rev. 53:171-216). Additional techniques for conjugating prophylactic or therapeutic moieties to antibodies are well known, see, e.g., Arnon et al., “Monoclonal Antibodies For Immunotargeting Of Drugs In Cancer Therapy,” in Monoclonal Antibodies And Cancer Therapy, Reisfeld et al. (eds.), pp. 243-56 (Alan R. Liss, Inc. 1985); Hellstrom et al., “Antibodies For Drug Delivery,” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review,” in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy,” in Monoclonal Antibodies For Cancer Detection And Therapy, Baldwin et al. (eds.), pp. 303-16 (Academic Press 1985), and Thorpe et al., 1982, Immunol. Rev. 62:119-58. Methods for fusing or conjugating antibodies to polypeptide moieties are known in the art. See, e.g., U.S. Pat. Nos. 5,336,603, 5,622,929, 5,359,046, 5,349,053, 5,447,851, and 5,112,946; EP 307,434; EP 367,166; International Publication Nos. WO 96/04388 and WO 91/06570; Ashkenazi et al., 1991, PNAS 88: 10535-10539; Zheng et al., 1995, J. Immunol. 154:5590-5600; and Vil et al., 1992, PNAS 89:11337-11341. The fusion of an antibody to a moiety does not necessarily need to be direct, but may occur through linker sequences. Such linker molecules are commonly known in the art and described in Denardo et al., 1998, Clin Cancer Res. 4:2483-90; Peterson et al., 1999, Bioconjug. Chem. 10:553; Zimmerman et al., 1999, Nucl. Med. Biol. 26:943-50; Garnett, 2002, Adv. Drug Deliv. Rev. 53:171-216, each of which is incorporated herein by reference in its entirety.

Alternatively, an antibody can be conjugated to a second antibody to form an antibody heteroconjugate as described by Segal in U.S. Pat. No. 4,676,980, which is incorporated herein by reference in its entirety.

A conjugated agent's relative efficacy in comparison to the free agent can depend on a number of factors. For example, rate of uptake of the antibody-agent into the cell (e.g., by endocytosis), rate/efficiency of release of the agent from the antibody, rate of export of the agent from the cell, etc. can all effect the action of the agent. Antibodies used for targeted delivery of agents can be assayed for the ability to be endocytosed by the relevant cell type (i.e., the cell type associated with the disorder to be treated) by any method known in the art. Additionally, the type of linkage used to conjugate an agent to an antibody should be assayed by any method known in the art such that the agent action within the target cell is not impeded.

The prophylactic or therapeutic moiety or drug conjugated to an EphA2/EphrinA1 Modulator of the invention (e.g., an EphA2 or EphrinA1 antibody that immunospecifically binds to an EphA2 or EphrinA1 polypeptide or fragment thereof, respectively) should be chosen to achieve the desired prophylactic or therapeutic effect(s) for the treatment, management or prevention of a non-neoplastic hyperproliferative epithelial and/or endothelial cell disorder, including but not limited to a disorder associated with increased deposition of extracellular matrix components (e.g., collagen, proteoglycans, tenascin and fibronectin) and/or aberrant angiogenesis. Non-limiting examples of such disorders include cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis. A clinician or other medical personnel should consider the following when deciding on which therapeutic moiety or drug to conjugate to an antibody that immunospecifically binds to an EphA2 or EphrinA1 polypeptide or fragment thereof: the nature of the disease, the severity of the disease, and the condition of the subject.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Alternatively, any of the methods described above may be used to generate EphA2/EphrinA1 Modulators that are EphA2 or EphrinA1 fusion proteins (see Section 4.2.2, infra).

4.2.1.3 Methods Of Producing Antibodies

The antibodies that immunospecifically bind to an antigen can be produced by any method known in the art for the synthesis of antibodies, in particular, by chemical synthesis or preferably, by recombinant expression techniques.

Polyclonal antibodies immunospecific for an antigen can be produced by various procedures well-known in the art. For example, a human antigen can be administered to various host animals including, but not limited to, rabbits, mice, rats, etc. to induce the production of sera containing polyclonal antibodies specific for the human antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T Cell Hybridomas 563 681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a non-murine antigen and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

The present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with a non-murine antigen with myeloma cells and then screening the hybridomas resulting from the fusion for hybridoma clones that secrete an antibody able to bind to the antigen.

Antibody fragments which recognize specific particular epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; International application No. PCT/GB91/O1 134; International publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. Preferably, the vectors for expressing the VH or VL domains comprise an EF-1α promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use humanized antibodies or chimeric antibodies. Completely human antibodies and humanized antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International publication Nos. WO 98/46645, WO 98/50433, WO 98/24893, WO98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, 1995, Int. Rev. Immunol. 13:65 93. For a detailed discussion of this technology for producing human antibodies and human monoclonal antibodies and protocols for producing such antibodies, see, e.g., International publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,311,415, which are incorporated herein by reference in their entireties.

Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions (see, e.g., U.S. Pat. No. 5,585,089; and Riechmann et al., 1988, Nature 332:323, which are incorporated herein by reference in their entireties).

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immuoglobulin. A humanized antibody comprises substantially all of at least one, and typically two, variable domains (Fab, Fab′, F(ab′)₂, Fabc, Fv) in which all or substantially all of the CDR regions correspond to those of a non human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibit cytotoxic activity, and the class is typically IgG1. Where such cytotoxic activity is not desirable, the constant domain may be of the IgG2 class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework and CDR sequences, more often 90%, and most preferably greater than 95%. A humanized antibody can be produced using variety of techniques known in the art, including but not limited to, CDR grafting (see e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see e.g., European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973, each of which is incorporated herein by its entirety by reference), chain shuffling (see e.g., U.S. Pat. No. 5,565,332, which is incorporated herein in its entirety by reference), and techniques disclosed in, e.g., U.S. Pat. No. 6,407,213, U.S. Pat. No. 5,766,886, International Publication No. WO 9317105, Tan et al., J. Immunol. 169:1119 25 (2002), Caldas et al., Protein Eng. 13(5):353 60 (2000), Morea et al., Methods 20(3):267 79 (2000), Baca et al., J. Biol. Chem. 272(16):10678 84 (1997), Roguska et al., Protein Eng. 9(10):895 904 (1996), Couto et al., Cancer Res. 55 (23 Supp):5973s-5977s (1995), Couto et al., Cancer Res. 55(8):1717 22 (1995), Sandhu J S, Gene 150(2):409 10 (1994), and Pedersen et al., J. Mol. Biol. 235(3):959 73 (1994), each of which is incorporated herein in its entirety by reference. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323, which are incorporated herein by reference in their entireties.)

Further, the antibodies that immunospecifically bind to EphA2 or EphrinA1 or fragments thereof can, in turn, be utilized to generate anti-idiotype antibodies that “mimic” an antigen using techniques well known to those skilled in the art. (See, e.g., Greenspan & Bona, 1989, FASEB J 7(5):437-444; and Nissinoff, 1991, J. Immunol. 147(8):2429-2438).

4.2.2 EphA2 and EphrinA1 Fragments and EphrinA1 Fragments as EphA2/EphrinA1 Modulators

In one embodiment, an EphA2/EphrinA1 Modulator of the invention is an EphA2 polypeptide. In a specific embodiment, an EphA2/Ephrin Modulator is a fragment of EphA2 (“EphA2 Fragments”). In accordance with this embodiment, the EphA2 Fragment preferably retains the ability to bind to EphrinA1. In a preferred embodiment, the EphA2 Fragment retains the ability to bind to EphrinA1 and inhibits or reduces binding of endogenous EphA2 to an endogenous ligand of EphA2, preferably EphrinA1. In a specific embodiment, an EphA2/Ephrin Modulator is an EphA2 Fragment that specifically binds to EphrinA1 or fragments thereof and does not bind to other Ephrin molecules or fragments thereof.

Non-limiting examples of EphA2 Fragments include, but are not limited to, EphA2 Fragments comprising the ligand binding domain of human EphA2 (amino acid residues 28 to 201) and any one or more of the following domains: the first fibronectin Type III domain (amino acid residues 332 to 424); the second fibronectin Type III domain (amino acid residues 439 to 519); the tyrosine kinase catalytic domain (amino acid residues 607 to 874); and/or the sterile alpha motif “SAM” domain (amino acid residues 902 to 968), the sequences of which may be found in the GenBank database (e.g., GenBank Accession No. NP_{—}004422.2 for human EphA2) In a specific embodiment, an EphA2 Fragment is soluble (i.e., not membrane-bound). In another specific embodiment, an EphA2 Fragment of the invention lacks the transmembrane domain of EphA2 (e.g., from amino acid residues 520 to 606) and is not membrane-bound. In further embodiments, an EphA2 Fragment of the invention comprises the extracellular domain or a fragment thereof and lacks the transmembrane domain or a fragment thereof such that the EphA2 is not membrane-bound. In yet further embodiments, an EphA2 Fragment of the invention comprises the cytoplasmic domain or a fragment of the cytoplasmic domain of EphA2 and lacks the transmembrane domain or a fragment thereof such that the EphA2 is not membrane-bound. In a specific embodiment, an EphA2 Fragment comprises only the extracellular domain of EphA2 or a fragment thereof. In another specific embodiment, an EphA2 Fragment comprises only the ligand binding domain (e.g., amino acid residues 28 to 201 of human EphA2 as disclosed in GenBank Accession No. NP_{—}004422.2). In specific embodiments, an EphA2 Fragment of the invention comprises specific fragments of the extracellular domain of human of EphA2 (e.g., amino acid residues 1 to 25, 1 to 50, 1 to 75, 1 to 100, 1 to 125, 1 to 150, 1 to 175, 1 to 200, 1 to 225, 1 to 250, 1 to 275, 1 to 300, 1 to 325, 1 to 350, 1 to 375, 1 to 400, 1 to 425, 1 to 450, 1 to 475, 1 to 500, or 1 to 525). In another specific embodiment, an EphA2 Fragment of the invention lacks the transmembrane domain of EphA2 such that the EphA2 is not membrane-bound.

The EphA2 Fragments include polypeptides that are 100%, 98%, 95%, 90%, 85%, 80%, 75%, 70%, 65%, 60%, 55%, 50%, 45%, 40% identical to endogenous EphA2 sequences. The determination of percent identity of two amino acid sequences can be determined by any method known to one skilled in the art, including BLAST protein searches. In specific embodiments, EphA2 Fragments of the invention can be analogs or derivatives of EphA2. For example, EphA2 Fragments of the invention include derivatives that are modified, i.e., by covalent attachment of any type of molecule to the polypeptide. For example, but not by way of limitation, the polypeptide derivatives (e.g., EphA2 polypeptide derivatives) include polypeptides that have been modified, e.g., by glycosylation, acetylation, pegylation, phosphorylation, amidation, derivatization by known protecting/blocking groups, proteolytic cleavage, linkage to a cellular ligand, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids. See also Section 4.2.3, infra.

In a specific embodiment, an EphA2/EphrinA1 Modulator of the invention is a dominant negative form of EphA2 which lacks the cytoplasmic domain or a portion thereof required for signaling. In a specific embodiment, the dominant negative form of EphA2 retains the ability to bind EphrinA1 but is incapable of signaling, induces low to negligible signaling or does not induce all the signal transduction pathways activated upon ligand-receptor interaction. In specific embodiments, low to negligible signaling in the context of EphA2 refers to a decrease in any aspect of EphA2 signaling upon ligand binding by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control in an in vivo and/or an in vitro assay described herein or well known to one of skill in the art. In certain aspects of the invention, EphA2 signaling encompasses any one or more of the signaling pathways that are activated upon EphA2 binding to its endogenous ligand (e.g., EphrinA1). Non-limiting examples of such signaling pathways include but are not limited to, the mitogen-activated protein kinase (MAPK)/ERK pathway, the Ras pathway, and pathways involving the Src family of kinases (for other Eph receptor pathways, see, Cheng et al., 2002, Cytokine & Growth Factor Rev. 13:75-85; Kullander and Klein, 2002, Nature Rev. 3:475-486; Holder and Klein, 1999, Development 126:2033-2044; Zhou, 1998, Pharmacol. Ther. 77:151-181; and Nakamoto and Bergemann, 2002, Microscopy Res. & Technique 59:58-67, which are all incorporated by reference herein in their entireties).

Various assays known to one of skill in the art may be performed to measure EphA2 signaling. For example, EphA2 phosphorylation may be measured to determine whether EphA2 signaling is activated upon ligand binding by measuring the amount of phosphorylated EphA2 present in EphrinA1-treated cells relative to control cells that are not treated with EphrinA1. EphA2 may be isolated using any protein immunoprecipitation method known to one of skill in the art and an EphA2 antibody of the invention. Phosphorylated EphA2 may then be measured using anti-phosphotyrosine antibodies (Upstate Tiotechnology, Inc., Lake Placid, N.Y.) using any standard immunoblotting method known to one of skill in the art. See, e.g., Cheng et al., 2002, Cytokine & Growth Factor Rev. 13:75-85. In another embodiment, MAPK phosphorylation may be measured to determine whether EphA2 signaling is activated upon ligand binding by measuring the amount of phosphorylated MAPK present in EphrinA1-treated cells relative to control cells that are not treated with EphrinA1 using standard immunoprecipitation and immunoblotting assays known to one of skill in the art (see, e.g., Miao et al., 2003, J. Cell Biol. 7:1281-1292, which is incorporated by reference herein in its entirety).

In one embodiment, an EphA2/EphrinA1 Modulator is an EphrinA1 polypeptide. In a specific embodiment, an EphA2/EphrinA1 Modulator of the invention is a fragment of EphrinA1 (“EphrinA1 Fragment”). In accordance with this embodiment, the EphrinA1 Fragment preferably retains the ability to bind to EphA2. In a preferred embodiment, the EphrinA1 Fragment retains the ability to bind to EphA2 and inhibits or reduces binding of endogenous EphrinA1 to endogenous EphA2.

Non-limiting examples of EphrinA1 Fragments include, but are not limited to, any fragment of human EphrinA1 as disclosed in the GenBank database (e.g., GenBank Accession Nos. NP_{—}004419 (variant 1) and NP_{—}872626 (variant 2)). In a specific embodiment, an EphrinA1 Fragment is soluble (i.e., not membrane-bound). In a specific embodiment, an EphrinA1 Fragment of the invention comprises the extracellular domain of human EphrinA1 or a fragment thereof. In further embodiments, an EphrinA1 Fragment of the invention comprises the extracellular domain of human EphrinA1 or a fragment thereof and is not membrane-bound. In specific embodiments, an EphrinA1 Fragment of the invention comprises specific fragments of the extracellular domain of human EphrinA1 variant 1 or a fragment thereof and is not membrane bound. In other specific embodiments, an EphrinA1 Fragment of the invention comprises specific fragments of the extracellular domain of human EphrinA1 variant 2 or a fragment thereof and is not membrane-bound.

The EphrinA1 Fragments include polypeptides that are 100%, 98%, 95%, 90%, 85%, 80%, 75%, 70%, 65%, 60%, 55%, 50%, 45%, 40% identical to endogenous EphrinA1 sequences. The determination of percent identity of two amino acid sequences can be determined by any method known to one skilled in the art, including BLAST protein searches. In specific embodiments, EphrinA1 Fragments of the invention can be analogs or derivatives of EphrinA1. For example, EphrinA1 Fragments of the invention include derivatives that are modified, i.e., by covalent attachment of any type of molecule to the polypeptide. For example, but not by way of limitation, the polypeptide derivatives (e.g., EphrinA1 polypeptide derivatives) include polypeptides that have been modified, e.g., by glycosylation, acetylation, pegylation, phosphorylation, amidation, derivatization by known protecting/blocking groups, proteolytic cleavage, linkage to a cellular ligand, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids. See also Section 4.2.3, infra.

In a specific embodiment, an EphA2/EphrinA1 Modulator is an EphA2 or EphrinA1 fusion protein. EphA2/EphrinA1 Modulators that are fusion proteins are discussed in further detail, for example, in Section 4.2.1.1, supra. In a preferred embodiment, an EphA2 or EphrinA1 fusion protein is soluble. Non-limiting examples of EphA2 fusion proteins include soluble forms of EphA2 such as EphA2-Fc (see, e.g., Cheng et al., 2002, Mol. Cancer Res. 1:2-11, which is incorporated by reference herein in its entirety). In a specific embodiment, an EphA2 fusion protein comprises EphA2 fused to the Fc portion of human immunoglobulin IgG1. In another embodiment, an EphA2 fusion protein comprises an EphA2 Fragment which retains its ability to bind EphrinA1 (e.g., the extracellular domain of EphA2) fused to the Fc portion of human immunoglobulin IgG1 (see, e.g., Carles-Kinch et al., 2002, Cancer Res. 62:2840-2847; and Cheng et al., 2002, Mol. Cancer Res. 1:2-11, which are incorporated by reference herein in their entireties). In yet a further embodiment, an EphA2 fusion protein comprises an EphA2 Fragment which retains its ability to bind EphrinA1 fused to a heterologous protein (e.g., human serum albumin).

Non-limiting examples of EphrinA1 fusion proteins include soluble forms of EphrinA1 such as EphrinA1-Fc (see, e.g., Duxbury et al., 2004, Biochem. & Biophys. Res. Comm. 320:1096-1102, which is incorporated by reference herein in its entirety). In a specific embodiment, an EphrinA1 fusion protein comprises EphrinA1 fused to an the Fc domain of human immunoglobulin IgG. In another embodiment, an EphrinA1 fusion protein comprises an EphrinA1 Fragment which retains its ability to bind EphA2 fused to the Fc domain of human immunoglobulin IgG. In yet a further embodiment, an EphrinA1 fusion protein comprises an EphrinA1 Fragment which retains its ability to bind EphA2 fused to a heterologous protein (e.g., human serum albumin).

Fragments of EphA2 or EphrinA1 can be made and assayed for the ability to bind EphrinA1 or EphA2, respectively, using biochemical, biophysical, genetic, and/or computational techniques for studying protein-protein interactions that are described herein or by any method known in the art. Non-limiting examples of methods for detecting protein binding (e.g., for detecting EphA2 binding to EphrinA1), qualitatively or quantitatively, in vitro or in vivo, include GST-affinity binding assays, far-Western Blot analysis, surface plasmon resonance (SRP), fluorescence resonance energy transfer (FRET), fluorescence polarization (FP), isothermal titration calorimetry (ITC), circular dichroism (CD), protein fragment complementation assays (PCA), various two-hybrid systems, and proteomics and bioinformatics-based approaches, such as the Scansite program for computational analysis (see, e.g., Fu, H., 2004, Protein-Protein Interactions: Methods and Applications (Humana Press, Totowa, N.J.); and Protein-Protein Interactions: A Molecular Cloning Manual, 2002, Golemis, ed. (Cold Spring Harbor Laboratory Press, Cold Spring Harbor, N.Y.) which are incorporated by reference herein in their entireties).

4.2.3.1 Polynucleotides Encoding Polypeptide EphA2/EphrinA1 Modulators

The EphA2/EphrinA1 Modulators of the invention include polypeptides produced from polynucleotides that hybridize to polynucleotides which encode polypeptides disclosed in sections 4.2.1 and 4.2.2 above. In one embodiment, antibodies of the invention include EphA2 or EphrinA1 monoclonal antibodies produced from polynucleotides that hybridize to polynucleotides encoding monoclonal antibodies that modulate the expression and/or activity EphA2 and/or EphrinA1 in one or more of the assays described in Section 4.6. In another embodiment, EphA2 Fragments or EphrinA1 Fragments used in the methods of the invention include polypeptides produced from polynucleotides that hybridize to polynucleotides encoding a fragments of EphA2 or EphrinA1. Conditions for hybridization include, but are not limited to, stringent hybridization conditions such as hybridization to filter-bound DNA in 6× sodium chloride/sodium citrate (SSC) at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 60° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3).

The EphA2/EphrinA1 Modulators of the invention include polynucleotides encoding polypeptides described herein. The polynucleotides encoding the polypeptides described herein (e.g., the antibodies of the invention or the EphA2 Fragments and EphrinA1 Fragments) may be obtained and sequenced by any method known in the art. For example, a polynucleotide encoding a polypeptide EphA2/EphrinA1 Modulator used in the methods of the invention may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the polypeptide, annealing and ligating of those oligonucleotides, and then amplification of the ligated oligonucleotides by PCR.

Alternatively, a polynucleotide encoding polypeptide EphA2/EphrinA1 Modulator used in the methods of the invention may be generated from nucleic acid from a suitable source. If a clone containing a nucleic acid encoding a particular polypeptide is not available, but the sequence of the polypeptide is known, a nucleic acid encoding the polypeptide may be chemically synthesized or obtained from a suitable source (e.g., an antibody cDNA library, or a cDNA library generated from, or nucleic acid, preferably poly A+ RNA, isolated from, any tissue or cells expressing the desired polypeptide, such as hybridoma cells selected to express an antibody of the invention or epithelial and/or endothelial cells that express EphA2 or EphrinA1) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the polypeptide EphA2/EphrinA1 Modulator. Amplified nucleic acids generated by PCR may then be cloned into replicable cloning vectors using any method well known in the art.

Once the nucleotide sequence of the polypeptide EphA2/EphrinA1 Modulator used in the methods of the invention is determined, the nucleotide sequence may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate polypeptides having a different amino acid sequence, for example to create amino acid substitutions, deletions, and/or insertions.

Standard techniques known to those skilled in the art can be used to introduce mutations in the nucleotide sequence encoding a polypeptide EphA2/EphrinA1 Modulator including, e.g., site-directed mutagenesis and PCR-mediated mutagenesis, which results in amino acid substitutions. Preferably, the derivatives include less than 15 amino acid substitutions, less than 10 amino acid substitutions, less than 5 amino acid substitutions, less than 4 amino acid substitutions, less than 3 amino acid substitutions, or less than 2 amino acid substitutions relative to the original EphA2/EphrinA1 Modulator. In a preferred embodiment, the derivatives have conservative amino acid substitutions made at one or more predicted non-essential amino acid residues.

The present invention also encompasses the use of antibodies or antibody fragments comprising the amino acid sequence of any EphA2 or EphrinA1 antibodies with mutations (e.g., one or more amino acid substitutions) in the framework or variable regions. Preferably, mutations in these antibodies maintain or enhance the avidity and/or affinity of the antibodies for the particular antigen(s) to which they immunospecifically bind. Standard techniques known to those skilled in the art (e.g., immunoassays or ELISA assays) can be used to assay the degree of binding between a polypeptide EphA2/EphrinA1 Modulator and its binding partner. In a specific embodiment, when a polypeptide EphA2/EphrinA1 Modulator is an antibody, an EphA2 Fragment, an EphrinA1 Fragment, an EphA2 fusion protein, an EphrinA1 fusion protein or a dominant negative form of EphA2, binding to EphA2 or EphrinA1, as appropriate, can be assessed.

4.2.3.2 Recombinant Production of Polypeptide EphA2/EphrinA1 Modulators

Recombinant expression of a polypeptide EphA2/EphrinA1 Modulator (including, but not limited to derivatives, analogs or fragments thereof) requires construction of an expression vector containing a polynucleotide that encodes the polypeptide. Once a polynucleotide encoding a polypeptide EphA2/EphrinA1 Modulator has been obtained, a vector for the production of the polypeptide EphA2/EphrinA1 Modulator may be produced by recombinant DNA technology using techniques well known in the art. Methods which are well known to those skilled in the art can be used to construct expression vectors containing polypeptide coding sequences and appropriate transcriptional and translational control signals. Thus, methods for preparing a protein by expressing a polynucleotide containing are described herein. These methods include, for example, in vitro recombinant DNA techniques, synthetic techniques, and in vivo genetic recombination. The invention, thus, provides replicable vectors comprising a nucleotide sequence encoding an polypeptide EphA2/EphrinA1 Modulator.

The expression vector is transferred to a host cell by conventional techniques and the transfected cells are then cultured by conventional techniques to produce a polypeptide EphA2/EphrinA1 Modulator. Thus, the invention includes host cells containing a polynucleotide encoding a polypeptide EphA2/EphrinA1 Modulator operably linked to a heterologous promoter.

A variety of host-expression vector systems may be utilized to express polypeptide EphA2/EphrinA1 Modulator (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express a polypeptide EphA2/EphrinA1 Modulator of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing polypeptide EphA2/EphrinA1 Modulator coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant polypeptide EphA2/EphrinA1 Modulator, are used for the expression of a polypeptide EphA2/EphrinA1 Modulator. For example, mammalian cells such as Chinese hamster ovary cells (CHO), in conjunction with a vector such as the major intermediate early gene promoter element from human cytomegalovirus is an effective expression system for polypeptide EphA2/EphrinA1 Modulators, especially antibody polypeptide EphA2/EphrinA1 Modulators (Foecking et al., 1986, Gene 45:101; and Cockett et al., 1990, BioTechnology 8:2). In a specific embodiment, the expression of nucleotide sequences encoding a polypeptide EphA2/EphrinA1 Modulator is regulated by a constitutive promoter, inducible promoter or tissue specific promoter.

In bacterial systems, a number of expression vectors may be advantageously selected depending upon the use intended for the polypeptide being expressed. For example, when a large quantity of such a protein is to be produced, for the generation of pharmaceutical compositions, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the polypeptide coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the polypeptide EphA2/EphrinA1 Modulator in infected hosts (e.g., see Logan & Shenk, 1984, PNAS 8 1:355-359). Specific initiation signals may also be required for efficient translation of inserted polypeptide coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 3T3, W138, BT483, Hs578T, HTB2, BT20 and T47D, NSO (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the polypeptide EphA2/EphrinA1 Modulator. Such engineered cell lines may be particularly useful in screening and evaluation of compositions that interact directly or indirectly with the polypeptide EphA2/EphrinA1 Modulator.

A number of selection systems may be used, including but not limited to, the herpes simplex virus thymidine kinase (Wigler et al., 1977, Cell 11:223), glutamine synthetase, hypoxanthine guanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, gs-, hgprt- or aprt-cells, respectively. Also, antimetabolite resistance can be used as the basis of selection for the following genes: dhfr, which confers resistance to methotrexate (Wigler et al., 1980, PNAS 77:357; O'Hare et al., 1981, PNAS 78:1527); gpt, which confers resistance to mycophenolic acid (Mulligan & Berg, 1981, PNAS 78:2072); neo, which confers resistance to the aminoglycoside G-418 (Wu and Wu, 1991, Biotherapy 3:87; Tolstoshev, 1993, Ann. Rev. Pharmacol. Toxicol. 32:573; Mulligan, 1993, Science 260:926; and Morgan and Anderson, 1993, Ann. Rev. Biochem. 62: 191; May, 1993, TIB TECH 11:155-); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150: 1, which are incorporated by reference herein in their entireties.

The expression levels of a polypeptide EphA2/EphrinA1 Modulator can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing polypeptide EphA2/EphrinA1 Modulator is amplifiable, increase in the level of inhibitor present in culture of host cell will increase the number of copies of the marker gene. Since the amplified region is associated with the polypeptide EphA2/EphrinA1 Modulator gene, production of the polypeptide EphA2/EphrinA1 Modulator will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, PNAS 77:2197). The coding sequences for the heavy and light chains may comprise cDNA or genomic DNA.

Once a polypeptide EphA2/EphrinA1 Modulator of the invention has been produced by recombinant expression, it may be purified by any method known in the art for purification of a polypeptide, for example, by chromatography (e.g., ion exchange, affinity, and sizing column chromatography), centrifugation, differential solubility, or by any other standard technique for the purification of proteins. Further, the polypeptide EphA2/EphrinA1 Modulators may be fused to heterologous polypeptide sequences described herein or otherwise known in the art to facilitate purification.

Polypeptide EphA2/EphrinA1 Modulators of the invention that are antibodies may be expressed using vectors which already include the nucleotide sequence encoding the constant region of the antibody molecule (see, e.g., U.S. Pat. Nos. 5,919,900; 5,747,296; 5,789,178; 5,591,639; 5,658,759; 5,849,522; 5,122,464; 5,770,359; 5,827,739; International Patent Publication Nos. WO 89/01036; WO 89/10404; Bebbington et al., 1992, BioTechnology 10:169). The variable domain of the antibody may be cloned into such a vector for expression of the entire heavy, the entire light chain, or both the entire heavy and light chains. In preferred embodiments for the expression of double-chained antibodies, vectors encoding both the heavy and light chains may be co-expressed in the host cell for expression of the entire immunoglobulin molecule.

In a specific embodiment, the expression of a polypeptide EphA2/EphrinA1 Modulator of the invention (e.g., an EphA2 or EphrinA1 peptide, polypeptide, protein or a fusion protein) is regulated by a constitutive promoter. In another embodiment, the expression of a polypeptide EphA2/EphrinA1 Modulator of the invention (e.g., an EphA2 or EphrinA1 peptide, polypeptide, protein or a fusion protein) is regulated by an inducible promoter. In another embodiment, the expression of a polypeptide EphA2/EphrinA1 Modulator of the invention (e.g., an EphA2 or EphrinA1 peptide, polypeptide, protein or a fusion protein) is regulated by a tissue-specific promoter. For example, EphA2 is regulated by Hoxal And Hoxb1 Homeobox transcription factors (see, e.g., Chen et al., 1998, J. Biol. Chem. 273:24670-24675, which is incorporated by reference herein in its entirety, and EphrinA1 is regulated by the Homeobox transcription factor HoxB3 (see, e.g., Myers et al., 2000, J. Cell Biol. 148:343-351, which is incorporated by reference herein in its entirety).

In one embodiment, the method of the invention comprises administration of a composition comprising nucleic acids encoding IL-9 antagonists or another prophylactic or therapeutic agent of the invention, said nucleic acids being part of an expression vector that expresses the IL-9 antagonist, another prophylactic or therapeutic agent of the invention, or fragments or chimeric proteins or heavy or light chains thereof in a suitable hostIn particular, such nucleic acids have promoters, preferably heterologous promoters, operably linked to the antibody coding region, said promoter being inducible or constitutive, and, optionally, tissue-specific.

4.3 Polynucleotide EphA2/EphrinA1 Modulators

In addition to the polypeptide EphA2/EphrinA1 Modulators of the invention, nucleic acid molecules can be used in methods of the invention. In one embodiment, a nucleic acid molecule EphA2/EphrinA1 Modulator can encode all or a portion of EphA2 to increase EphA2 expression or availability for ligand (preferably, EphrinA1) binding. In another embodiment, a nucleic acid molecule EphA2/EphrinA1 Modulator can encode all or a portion of EphrinA1 to increase the amount of EphrinA1 available for binding to EphA2. Any method known in the art can be used to increase expression of EphA2 or EphrinA1 using nucleic acid molecules. In a further embodiment, a nucleic acid EphA2/EphrinA1 Modulator reduces the amount of endogenous EphA2 available for ligand binding to EphrinA1. In yet a further embodiment, a nucleic acid molecule EphA2/EphrinA1 Modulator reduces the amount of EphrinA1 available for binding to EphA2. Any method known in the art to decrease expression of EphA2 or EphrinA1 can be used in the methods of the invention including, but not limited to, antisense and RNA interference technology. Thus, EphA2/EphrinA1 Modulators encompasses those agents that serve to increase or decrease EphrinA1 expression or availability for EphA2-binding, and those agents that serve to increase or decrease EphA2 expression or availability for binding to an endogenous EphA2 ligand (preferably, EphrinA1).

4.3.1 Antisense

The present invention encompasses EphA2 and EphrinA1 antisense nucleic acid molecules, i.e., molecules which are complementary to all or part of a sense nucleic acid encoding EphA2 or EphrinA1, molecules which are complementary to the coding strand of a double-stranded EphA2 or EphrinA1 cDNA molecule or molecules complementary to an EphA2 or EphrinA1 mRNA sequence. EphA2 and EphrinA1 antisense nucleic acid molecules can be produced by any method known to those skilled in the art, using the human EphA2 and EphrinA1 mRNA sequences disclosed, for example, in the GenBank database.

In a specific embodiment, an EphA2 antisense nucleic acid molecule may be produced using the human EphA2 mRNA sequence disclosed in GenBank Accession No. NM_{—}004431.2. Examples of EphA2 antisense nucleic acid molecules are also disclosed, e.g., in Cheng et al., 2002, Mol. Cancer Res. 1:2-11 and in Carles-Kinch et al., 2002, Cancer Res. 62:2840-2847, which are both incorporated by reference herein in their entireties. In a specific embodiment, an EphA2 antisense nucleic acid molecule can be complementary to any of the following regions (or a portion thereof) of human EphA2 as encoded by the coding strand or sense strand of human EphA2: the ligand binding domain, the transmembrane domain, the first fibronectin type III domain, the second fibronectin type III domain, the tyrosine kinase domain, or the SAM domain.

In a specific embodiment, an EphA2 antisense nucleic acid molecule is not 5′-CCAGCAGTACCACTTCCTTGCCCTGCGCCG-3′ (SEQ ID NO:40) and/or 5′-GCCGCGTCCCGTTCCTTCACCATGACGACC-3′ (SEQ ID NO:41). In another specific embodiment, an EphA2 antisense nucleic acid moleucle is not 5′-CCAGCAGTACCGCTTCCTTGCCCTGCGGCCG-3′ (SEQ ID NO:42) and/or 5′-GCCGCGTCCCGTTCCTTCACCATGACGACC-3′ (SEQ ID NO:43). In certain embodiments, an EphA2/EphrinA1 Modulator of the invention is not an EphA2 antisense nucleic acid molecule.

In a preferred embodiment, an antisense EphA2/EphrinA1 Modulator of the invention is a human EphrinA1 antisense nucleic acid molecule. In a specific embodiment, a human EphrinA1 antisense nucleic acid molecule may be produced using the human EphrinA1 mRNA sequence disclosed in Genbank Accession No. BC032698. Examples of EphrinA1 antisense nucleic acid molecules are disclosed, e.g., in Potla et al., 2002, Cancer Lett. 175(2):187-95, which is incorporated by reference herein in its entirety. In a specific embodiment, an EphrinA1 antisense nucleic acid molecule of the invention is not the EphrinA1 antisense nucleic acid molecule(s) disclosed in Potla et al., 2002, Cancer Lett. 175(2):187-95. In certain embodiments, the EphA2/EphrinA1 Modulator of the invention is not an EphrinA1 antisense nucleic acid molecule.

An antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides (e.g., phosphorothioate-modified) designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, i.e., EphrinA1).

The antisense nucleic acid molecules of the invention are typically administered to a subject or generated in situ such that they hybridize with or bind to cellular mRNA and/or genomic DNA encoding a selected polypeptide of the invention to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al., 1987, Nucleic Acids Res. 15:6625). The antisense nucleic acid molecule can also comprise a 2′-o-methylribonucleotide (Inoue et al., 1987, Nucleic Acids Res. 15:6131) or a chimeric RNA-DNA analogue (Inoue et al., 1987, FEBS Lett. 215:327).

4.3.2 RNA Interference

In certain embodiments, an RNA interference (RNAi) molecule is used to decrease EphrinA1 expression. In other embodiments, an RNAi molecule is used to decrease EphA2 expression. RNAi is defined as the ability of double-stranded RNA (dsRNA) to suppress the expression of a gene corresponding to its own sequence. RNAi is also called post-transcriptional gene silencing or PTGS. Since the only RNA molecules normally found in the cytoplasm of a cell are molecules of single-stranded mRNA, the cell has enzymes that recognize and cut dsRNA into fragments containing 21-25 base pairs (approximately two turns of a double helix). The antisense strand of the fragment separates enough from the sense strand so that it hybridizes with the complementary sense sequence on a molecule of endogenous cellular mRNA (e.g., human EphrinA1 mRNA sequence at Genbank Accession No. BC032698). This hybridization triggers cutting of the mRNA in the double-stranded region, thus destroying its ability to be translated into a polypeptide. Introducing dsRNA corresponding to a particular gene thus knocks out the cell's own expression of that gene in particular tissues and/or at a chosen time.

Double-stranded (ds) RNA can be used to interfere with gene expression in mammals (Wianny & Zernicka-Goetz, 2000, Nature Cell Biology 2: 70-75; incorporated herein by reference in its entirety). dsRNA is used as inhibitory RNA or RNAi of the function of EphrinA1 to produce a phenotype that is the same as that of a null mutant of EphrinA1 (Wianny & Zernicka-Goetz, 2000, Nature Cell Biology 2: 70-75). In certain embodiments, dsDNA encoding dsRNA (e.g., as hairpin structures) is used to express RNAi-mediating dsDNA in the cell.

4.3.2 Aptamers as EphA2/EphrinA1 Modulators

In specific embodiments, the invention provides aptamers of EphA2 and EphrinA1. As is known in the art, aptamers are macromolecules composed of nucleic acid (e.g., RNA, DNA) that bind tightly to a specific molecular target (e.g., EphA2 or EphrinA1 proteins, EphA2 or EphrinA1 polypeptides and/or EphA2 or EphrinA1 epitopes as described herein). A particular aptamer may be described by a linear nucleotide sequence and is typically about 15-60 nucleotides in length. The chain of nucleotides in an aptamer form intramolecular interactions that fold the molecule into a complex three-dimensional shape, and this three-dimensional shape allows the aptamer to bind tightly to the surface of its target molecule. Given the extraordinary diversity of molecular shapes that exist within the universe of all possible nucleotide sequences, aptamers may be obtained for a wide array of molecular targets, including proteins and small molecules. In addition to high specificity, aptamers have very high affinities for their targets (e.g., affinities in the picomolar to low nanomolar range for proteins). Aptamers are chemically stable and can be boiled or frozen without loss of activity. Because they are synthetic molecules, they are amenable to a variety of modifications, which can optimize their function for particular applications. For in vivo applications, aptamers can be modified to dramatically reduce their sensitivity to degradation by enzymes in the blood. In addition, modification of aptamers can also be used to alter their biodistribution or plasma residence time.

Selection of aptamers that can bind to EphA2 or EphrinA1 or a fragment there of can be achieved through methods known in the art. For example, aptamers can be selected using the SELEX (Systematic Evolution of Ligands by Exponential Enrichment) method (Tuerk and Gold, 1990, Science 249:505-510, which is incorporated by reference herein in its entirety). In the SELEX method, a large library of nucleic acid molecules (e.g., 10¹⁵ different molecules) is produced and/or screened with the target molecule (e.g., EphA2 or EphrinA1 proteins, EphA2 or EphrinA1 polypeptides and/or EphA2 or EphrinA1 epitopes or fragments thereof as described herein). The target molecule is allowed to incubate with the library of nucleotide sequences for a period of time. Several methods can then be used to physically isolate the aptamer target molecules from the unbound molecules in the mixture and the unbound molecules can be discarded. The aptamers with the highest affinity for the target molecule can then be purified away from the target molecule and amplified enzymatically to produce a new library of molecules that is substantially enriched for aptamers that can bind the target molecule. The enriched library can then be used to initiate a new cycle of selection, partitioning, and amplification. After 5-15 cycles of this selection, partitioning and amplification process, the library is reduced to a small number of aptamers that bind tightly to the target molecule. Individual molecules in the mixture can then be isolated, their nucleotide sequences determined, and their properties with respect to binding affinity and specificity measured and compared. Isolated aptamers can then be further refined to eliminate any nucleotides that do not contribute to target binding and/or aptamer structure (i.e., aptamers truncated to their core binding domain). See, e.g., Jayasena, 1999, Clin. Chem. 45:1628-1650 for review of aptamer technology, the entire teachings of which are incorporated herein by reference).

In particular embodiments, the aptamers of the invention have the binding specificity and/or functional activity described herein for the antibodies of the invention. Thus, for example, in certain embodiments, the present invention is drawn to aptamers that have the same or similar binding specificity as described herein for the antibodies of the invention (e.g., binding specificity for EphA2 or EphrinA1 polypeptide, fragments of vertebrate EphA2 or EphrinA1 polypeptides, epitopic regions of vertebrate EphA2 or EphrinA1 polypeptides (e.g., epitopic regions of EphA2 or EphrinA1 that are bound by the antibodies of the invention). In particular embodiments, the aptamers of the invention can bind to an EphA2 or EphrinA1 polypeptide and inhibit one or more activities of the EphA2 or EphrinA1 polypeptide.

4.4 Vaccines as EphA2/EphrinA1 Modulators

In a specific embodiment, an EphA2/EphrinA1 Modulator is an EphA2 and/or an EphrinA1 vaccine. As used herein, the term “EphA2 vaccine” can be any reagent that elicits or mediates an immune response against EphA2-expressing cells. In certain embodiments, an EphA2 vaccine is an EphA2 antigenic peptide of the invention, an expression vehicle (e.g., a naked nucleic acid or a viral or bacterial vector or a cell) for an EphA2 antigenic peptide (e.g., which delivers the EphA2 antigenic peptide), or T cells or antigen presenting cells (e.g., dendritic cells or macrophages) that have been primed with the EphA2 antigenic peptide of the invention. As used herein, the terms “EphA2 antigenic peptide” and “EphA2 antigenic polypeptide” refer to an EphA2 polypeptide, or a fragment, analog, or derivative thereof comprising one or more B cell epitopes or T cell epitopes of EphA2. The EphA2 polypeptide may be from any species. In certain embodiments, an EphA2 polypeptide refers to the mature, processed form of EphA2. In other embodiments, an EphA2 polypeptide refers to an immature form of EphA2. For a description of EphA2 vaccines, see, e.g., U.S. Provisional Application Ser. No. 60/556,601, entitled “EphA2 Vaccines,” filed Mar. 26, 2004; U.S. Provisional Application Ser. No. ______ filed Aug. 18, 2004, entitled “EphA2 Vaccines” (Attorney Docket No. 10271-136-888); U.S. Provisional Application Ser. No. ______ filed Oct. 1, 2004, entitled “EphA2 Vaccines” (Attorney Docket No. 10271-143-888); and U.S. Provisional Application Ser. No. ______ filed Oct. 7, 2004, entitled “EphA2 Vaccines” (Attorney Docket No. 10271-148-888), each of which is incorporated by reference herein in its entirety.

In a specific embodiment, an EphA2/EphrinA1 Modulator is an EphrinA1 vaccine. As used herein, the term “EphrinA1 vaccine” can be any reagent that elicits or mediates an immune response against EphrinA1 on EprhinA1-expressing cells. In certain embodiments, an EphrinA1 vaccine is an EphrinA1 antigenic peptide of the invention, an expression vehicle (e.g., a naked nucleic acid or a viral or bacterial vector or a cell) for an EphrinA1 antigenic peptide (e.g., which delivers the EphrinA1 antigenic peptide), or T cells or antigen presenting cells (e.g., dendritic cells or macrophages) that have been primed with the EphrinA1 antigenic peptide of the invention. As used herein, the terms “EphrinA1 antigenic peptide” and “EphrinA1 antigenic polypeptide” refer to an EphrinA1 polypeptide, or a fragment, analog, or derivative thereof comprising one or more B cell epitopes or T cell epitopes of EphrinA1. The EphrinA1 polypeptide may be from any species. In certain embodiments, an EphrinA1 polypeptide refers to the mature, processed form of EphrinA1. In other embodiments, an EphA2 polypeptide refers to an immature form of EphrinA1.

The present invention thus provides EphA2/EphrinA1 Modulator-based agents that are EphA2- and/or EphrinA1 antigenic peptide expression vehicles expressing an EphA2 or an EphrinA1 antigenic peptide that can elicit or mediate a cellular immune response, a humoral response, or both, against cells that overexpress EphA2 or EphrinA1. Where the immune response is a cellular immune response, it can be a Tc, Th1 or a Th2 immune response. In a preferred embodiment, the immune response is a Th2 cellular immune response. In another preferred embodiment, an EphA2 or an EphrinA1 antigenic peptide expressed by an EphA2-/EphrinA1-antigenic peptide expression vehicle is an EphA2 or EphrinA1 antigenic peptide that is capable of eliciting an immune response against EphA2- and/or EphrinA1-expressing cells involved in a disease or disorder associated with increased deposition of extracellular matrix components (e.g., collagen, proteoglycans, tenascin and fibronectin) and/or or aberrant (e.g., increased) angiogenesis. Non-limiting examples of such disorders include cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis.

In a specific embodiment, the EphA2- and/or EphrinA1 antigenic expression vehicle is a microorganism expressing an EphA2 and/or an EphrinA1 antigenic peptide. In a specific embodiment, the EphA2- and/or EphrinA1 antigenic expression vehicle is an attenuated bacteria. Non-limiting examples of bacteria include Listeria monocytogenes, include but are not limited to Borrelia burgdorferi, Brucella melitensis, Escherichia coli, enteroinvasive Escherichia coli, Legionella pneumophila, Salmonella typhi, Salmonella typhimurium, Shigella spp., Streptococcus spp., Treponema pallidum, Yersinia enterocohtica, Listeria monocytogenes, Mycobacterium avium, Mycobacterium bovis, Mycobacterium tuberculosis, BCG, Mycoplasma hominis, Rickettsiae quintana, Cryptococcus neoformans, Histoplasma capsulatum, Pneumocystis carnii, Eimeria acervulina, Neospora caninum, Plasmodium falciparum, Sarcocystis suihominis, Toxoplasma gondii, Leishmania amazonensis, Leishmania major, Leishmania mexacana, Leptomonas karyophilus, Phytomonas spp., Trypanasoma cruzi, Encephahtozoon cuniculi, Nosema helminthorum, Unikaryon legeri. In a specific embodiment, an EphA2/EphrinA1 Modulator vaccine is Listeria-based. As used herein, a Listeria-based vaccine expresses an EphA2 and/or an EphrinA1 antigenic peptide. In a further embodiment, the Listeria-based vaccine expressing an EphA2- and/or an EphrinA1 antigenic peptide is attenuated. In a specific embodiment, an EphA2/EphrinA1 Modulator vaccine is not Listeria-based or is not EphA2-based.

In another embodiment, the EphA2- and/or EphrinA1 antigenic peptide expression vehicle is a virus expressing an EphA2- and/or an EphrinA1 antigenic peptide. Non-limiting examples of viruses include RNA viruses (e.g., single stranded RNA viruses and double stranded RNA viruses), DNA viruses (e.g., double stranded DNA viruses), enveloped viruses, and non-enveloped viruses. Other non-limiting examples of viruses useful as EphA2- and/or EphrinA1 antigenic peptide expression vehicles include retroviruses (including but not limited to lentiviruses), adenoviruses, adeno-associated viruses, or herpes simplex viruses. Preferred viruses for administration to human subjects are attenuated viruses. A virus can be attenuated, for example, by exposing the virus to mutagens, such as ultraviolet irradiation or chemical mutagens, by multiple passages and/or passage in non-permissive hosts, and/or genetically altering the virus to reduce the virulence and pathogenicity of the virus.

Microorganisms can be produced by a number of techniques well known in the art. For example, antibiotic-sensitive strains of microorganisms can be selected, microorganisms can be mutated, and mutants that lack virulence factors can be selected, and new strains of microorganisms with altered cell wall lipopolysaccharides can be constructed. In certain embodiments, the microorganisms, can be attenuated by the deletion or disruption of DNA sequences which encode for virulence factors which insure survival of the microorganisms in the host cell, especially macrophages and neutrophils, by, for example, homologous recombination techniques and chemical or transposon mutagenesis. Many, but not all, of these studied virulence factors are associated with survival in macrophages such that these factors are specifically expressed within macrophages due to stress, for example, acidification, or are used to induced specific host cell responses, for example, macropinocytosis, Fields et al., 1986, Proc. Natl. Acad. Sci. USA 83:5189-5193. Bacterial virulence factors include, for example: cytolysin; defensin resistance loci; DNA K; fimbriae; GroEL; inv loci; lipoprotein; LPS; lysosomal fusion inhibition; macrophage survival loci; oxidative stress response loci; pho loci (e.g., PhoP and PhoQ); pho activated genes (pag; e.g., pagB and pagc); phoP and phoQ regulated genes (prg); porins; serum resistance peptide; virulence plasmids (such as spvB, traT and ty2).

Yet another method for the attenuation of the microorganisms is to modify substituents of the microorganism which are responsible for the toxicity of that microorganism. For example, lipopolysaccharide (LPS) or endotoxin is primarily responsible for the pathological effects of bacterial sepsis. The component of LPS which results in this response is lipid A (LA). Elimination or mitigation of the toxic effects of LA results in an attenuated bacteria since 1) the risk of septic shock in the patient would be reduced and 2) higher levels of the bacterial EphA2 or EphrinA1 antigenic peptide expression vehicle could be tolerated.

Rhodobacter (Rhodopseudomonas) sphaeroides and Rhodobacter capsulatus each possess a monophosphoryl lipid A (MLA) which does not elicit a septic shock response in experimental animals and, further, is an endotoxin antagonist. Loppnow et al., 1990, Infect. Immun. 58:3743-3750; Takayma et al., 1989, Infect. Immun. 57:1336-1338. Gram negative bacteria other than Rhodobacter can be genetically altered to produce MLA, thereby reducing its potential of inducing septic shock.

Yet another example for altering the LPS of bacteria involves the introduction of mutations in the LPS biosynthetic pathway. Several enzymatic steps in LPS biosynthesis and the genetic loci controlling them in a number of bacteria have been identified, and several mutant bacterial strains have been isolated with genetic and enzymatic lesions in the LPS pathway. In certain embodiments, the LPS pathway mutant is a firA mutant. firA is the gene that encodes the enzyme UDP-3-O(R-30 hydroxymyristoyl)-glycocyamine N-acyltransferase, which regulates the third step in endotoxin biosynthesis (Kelley et al., 1993, J. Biol. Chem. 268:19866-19874).

As a method of insuring the attenuated phenotype and to avoid reversion to the non-attenuated phenotype, the bacteria may be engineered such that it is attenuated in more than one manner, e.g., a mutation in the pathway for lipid A production and one or more mutations to auxotrophy for one or more nutrients or metabolites, such as uracil biosynthesis, purine biosynthesis, and arginine biosynthesis.

The EphA2 or EphrinA1 antigenic peptides are preferably expressed in a microorganism, such as bacteria, using a heterologous gene expression cassette. A heterologous gene expression cassette is typically comprised of the following ordered elements: (1) prokaryotic promoter; (2) Shine-Dalgarno sequence; (3) secretion signal (signal peptide); and, (4) heterologous gene. Optionally, the heterologous gene expression cassette may also contain a transcription termination sequence, in constructs for stable integration within the bacterial chromosome. While not required, inclusion of a transcription termination sequence as the final ordered element in a heterologous gene expression cassette may prevent polar effects on the regulation of expression of adjacent genes, due to read-through transcription.

The expression vectors introduced into the microorganism EphA2 or EphrinA1 vaccines are preferably designed such that microorganism-produced EphA2 or EphrinA1 peptides and, optionally, prodrug converting enzymes, are secreted by microorganism. A number of bacterial secretion signals are well known in the art and may be used in the compositions and methods of the present invention. In certain embodiments of the present invention, the bacterial EphA2 antigenic peptide expression vehicles are engineered to be more susceptible to an antibiotic and/or to undergo cell death upon administration of a compound. In other embodiments of the present invention, the bacterial EphA2 or EphrinA1 antigenic peptide expression vehicles are engineered to deliver suicide genes to the target EphA2- or EphrinA1-expressing cells. These suicide genes include pro-drug converting enzymes, such as Herpes simplex thymidine kinase (TK) and bacterial cytosine deaminase (CD). TK phosphorylates the non-toxic substrates acyclovir and ganciclovir, rendering them toxic via their incorporation into genomic DNA. CD converts the non-toxic 5-fluorocytosine (5-FC) into 5-fluorouracil (5-FU), which is toxic via its incorporation into RNA. Additional examples of pro-drug converting enzymes encompassed by the present invention include cytochrome p450 NADPH oxidoreductase which acts upon mitomycin C and porfiromycin (Murray et al., 1994, J. Pharmacol. Exp. Therapeut. 270:645-649). Other exemplary pro-drug converting enzymes that may be used include: carboxypeptidase; beta-glucuronidase; penicillin-V-amidase; penicillin-G-amidase; beta-lactamase; beta.-glucosidase; nitroreductase; and carboxypeptidase A.

Exemplary secretion signals that can be used with gram-positive microorganisms include SecA (Sadaie et al., 1991, Gene 98:101-105), SecY (Suh et al., 1990, Mol. Microbiol. 4:305-314), SecE (Jeong et al., 1993, Mol. Microbiol. 10:133-142), FtsY and FfH (PCT/NL 96/00278), and PrsA (International Publication No. WO 94/19471). Exemplary secretion signals that may be used with gram-negative microorganisms include those of soluble cytoplasmic proteins such as SecB and heat shock proteins; that of the peripheral membrane-associated protein SecA; and those of the integral membrane proteins SecY, SecE, SecD and SecF.

The promoters driving the expression of the EphA2 or EphrinA1 antigenic peptides and, optionally, pro-drug converting enzymes, may be either constitutive, in which the peptides or enzymes are continually expressed, inducible, in which the peptides or enzymes are expressed only upon the presence of an inducer molecule(s), or cell-type specific control, in which the peptides or enzymes are expressed only in certain cell types. For example, a suitable inducible promoter can be a promoter responsible for the bacterial “SOS” response (Friedberg et al., In: DNA Repair and Mutagenesis, pp. 407-455, Am. Soc. Microbiol. Press, 1995). Such a promoter is inducible by numerous agents including chemotherapeutic alkylating agents such as mitomycin (Oda et al., 1985, Mutation Research 147:219-229; Nakamura et al., 1987, Mutation Res. 192:239-246; Shimda et al., 1994, Carcinogenesis 15:2523-2529) which is approved for use in humans. Promoter elements which belong to this group include umuC, sulA and others (Shinagawa et al., 1983, Gene 23:167-174; Schnarr et al., 1991, Biochemie 73:423-431). The sulA promoter includes the ATG of the sulA gene and the following 27 nucleotides as well as 70 nucleotides upstream of the ATG (Cole, 1983, Mol. Gen. Genet. 189:400-404). Therefore, it is useful both in expressing foreign genes and in creating gene fusions for sequences lacking initiating codons.

In certain embodiments, an EphA2/EphrinA1 Modulator vaccine does not comprise a bacteria as an EphA2 and/or EphrinA1 antigenic peptide expression vehicle. In other embodiments, an EphA2/EphrinA1 Modulator is not an EphA2 vaccine and/or an EphrinA1 vaccine. In yet other embodiments, an EphA2/EphrinA1 Modulator is not an EphA2 and/or EphrinA1 antigenic peptide alone (i.e., without an expression vehicle).

4.5 Prophylactic/Therapeutic Methods

The present invention provides methods for treating, managing, or preventing a non-neoplastic hyperproliferative epithelial and/or endothelial cell disorder, including but not limited to disorders associated with increased deposition of ECM components (e.g., collagen, proteoglycans and fibronectin) and/or aberrant angiogenesis in a subject comprising administering one or more EphA2/EphrinA1 Modulators or cell proliferation stimulative agents of the invention. Non-limiting examples of such disorders include cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis. The present invention also provides methods for treating, managing or preventing a non-neoplastic hyperproliferative epithelial and/or endothelial cell disorder comprising administering, one or more EphA2/EphrinA1 Modulators and one or more other therapies (see Section 4.5.2, infra for examples of such therapies). Preferably, such other therapies are useful in the treatment, management, or prevention of non-neoplastic hyperproliferative epithelial and/or endothelial cell disorders including, but not limited to, disorders associated with increased deposition of ECM components and disorders associated with aberrant angiogenesis. In a specific embodiment, therapies other than EphA2/EphrinA1 Modulators that are useful in the treatment, prevention or management of cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis are used in combination with EphA2 or EphrinA1 Modulators in accordance with the invention.

The dosage amounts and frequencies of administration provided herein are encompassed by the terms effective amount, therapeutically effective and prophylactically effective. The dosage and frequency further will typically vary according to factors specific for each patient depending on the specific therapeutic or prophylactic agents administered, the severity and type of non-neoplastic hyperproliferative epithelial and/or endothelial cell disorder, the route of administration, as well as age, body weight, response, and the past medical history of the patient. Suitable regimens can be selected by one skilled in the art by considering such factors and by following, for example, dosages reported in the literature and recommended in the Physician's Desk Reference (58^{th} ed., 2004). See Section 4.7.3 for specific dosage amounts and frequencies of administration of the prophylactic and therapeutic agents provided by the invention.

4.5.1 Patient Population

The present invention encompasses methods for treating, managing, or preventing a non-neoplastic hyperproliferative epithelial and/or endothelial cell disorder, symptom thereof, in a subject comprising administering one or more EphA2/EphrinA1 Modulators of the invention. The subject is preferably a mammal such as non-primate (e.g., cows, pigs, horses, cats, dogs, rats, etc.) and a primate (e.g., monkey, such as a cynomolgous monkey and a human). In a specific embodiment, the subject is a non-human animal. In a preferred embodiment, the subject is a human.

The methods of the invention comprise the administration of one or more EphA2/EphrinA1 Modulators of the invention to patients suffering from or expected to suffer from (e.g., patients with a genetic predisposition for or patients that have previously suffered from) a non-neoplastic hyperproliferative epithelial and/or cell disorder. Such patients may have been previously treated or are currently being treated for the non-neoplastic hyperproliferative epithelial and/or endothelial cell disorder, e.g., with a non-EphA2/EphrinA1 Modulator therapy. In accordance with the invention, an EphA2/EphrinA1 Modulator may be used as any line of therapy, including, but not limited to, a first, second, third and fourth line of therapy. Further, in accordance with the invention, an EphA2/EphrinA1 Modulator can be used before any adverse effects or intolerance of the non-EphA2/EphrinA1 Modulator therapies occurs. The invention encompasses methods for administering one or more EphA2/EphrinA1 Modulators of the invention to prevent the onset or recurrence of a non-neoplastic hyperproliferative epithelial and/or endothelial cell disorder, including but not limited to cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis.

In one embodiment, the invention also provides methods of treatment or management of non-neoplastic hyperproliferative epithelial and/or endothelial cell or disorders as alternatives to current therapies. In a specific embodiment, the current therapy has proven or may prove too toxic (i.e., results in unacceptable or unbearable side effects) for the patient. In another embodiment, an EphA2/EphrinA1 Modulator decreases the side effects as compared to the current therapy. In another embodiment, the patient has proven refractory to a current therapy. In such embodiments, the invention provides for the administration of one or more EphA2/EphrinA1 Modulators of the invention without any other non-neoplastic hyperproliferative cell or excessive cell accumulation disorder therapies. In certain embodiments, one or more EphA2/EphrinA1 Modulators of the invention can be administered to a patient in need thereof instead of another therapy to treat non-neoplastic hyperproliferative epithelial and/or endothelial cell disorders.

The present invention also encompasses methods for administering one or more EphA2/EphrinA1 Modulators of the invention to treat or ameliorate symptoms of a non-neoplastic hyperproliferative epithelial and/or endothelial cell disorder in patients that are or have become refractory to non-EphA2/EphrinA1 Modulator therapies. The determination of whether the symptoms are refractory can be made either in vivo or in vitro by any method known in the art for assaying the effectiveness of a therapy on affected cells in the non-neoplastic hyperproliferative epithelial and/or endothelial cell disorder, particularly epithelial and/or endothelial cells, or in patients that are or have become refractory to non-EphA2/EphrinA1 Modulator therapies.

4.5.2 Other Prophylactic/Therapeutic Agents

The invention provides methods for treating, managing or preventing a non-neoplastic hyperproliferative epithelial and/or endothelial cell disorder by administering one or more EphA2/EphrinA1 Modulators of the invention in combination with one or more therapies. Preferably, those other therapies are currently being used or are useful in the treatment, management or prevention of a non-neoplastic hyperproliferative epithelial and/or endothelial cell disorder. In a specific embodiment, the invention provides a method of treating, managing or preventing a non-neoplastic hyperproliferative epithelial and/or endothelial cell disorder, the method comprising administering to a subject in need thereof an effective amount of an EphA2/EphrinA1 Modulator and an effective amount of a therapy other than an EphA2/EphrinA1 Modulator. Any therapy (e.g., prophylactic or therapeutic agents) which is known to be useful, or which has been used or is currently being used for the prevention, management, treatment or amelioration of a non-neoplastic hyperproliferative epithelial and/or endothelial cell disorder or a symptom thereof can be used in combination with an EphA2/EphrinA1 Modulator in accordance with the invention described herein. See, e.g., Gilman et al., Goodman and Gilman's: The Pharmacological Basis of Therapeutics, Tenth Ed., McGraw-Hill, New York, 2001; The Merck Manual of Diagnosis and Therapy, Berkow, M. D. et al. (eds.), 17^{th} Ed., Merck Sharp & Dohme Research Laboratories, Rahway, N.J., 1999; and Cecil Textbook of Medicine, 20^{th} Ed., Bennett and Plum (eds.), W.B. Saunders, Philadelphia, 1996, for information regarding therapies, in particular prophylactic or therapeutic agents, which have been or are currently being used for preventing, treating, managing, and/or ameliorating a non-neoplastic hyperproliferative epithelial and/or endothelial cell disorder or a symptom thereof. Therapeutic or prophylactic agents include, but are not limited to, small molecules, synthetic drugs, peptides, polypeptides, proteins, nucleic acids, (e.g., DNA and RNA nucleotides including, but not limited to, antisense nucleotide sequences, triple helices, RNAi, and nucleotide sequences encoding biologically active proteins, polypeptides or peptides) antibodies, synthetic or natural inorganic molecules, mimetic agents, and synthetic or natural organic molecules. Examples of prophylactic and therapeutic agents include, but are not limited to, immunomodulatory agents, anti-inflammatory agents (e.g., adrenocorticoids, corticosteroids, (e.g., beclomethasone, budesonide, flunisolide, fluticasone, triamcinolone, methylprednisolone, prednisolone, prednisone, hydrocortisone), glucocorticoids, steroids, and non-steroidal anti-inflammatory drugs (e.g., aspirin, ibuprofen, diclofenac, and COX-2 inhibitors), anticholinergic agents (e.g., ipratropium bromide and oxitropium bromide), sulphasalazine, penicillamine, dapsone, antihistamines, anti-malarial agents (e.g., hydroxychloroquine), anti-viral agents, and antibiotics (e.g., dactinomycin (formerly actinomycin), bleomycin, erythromycin, penicillin, mithramycin, and anthramycin (AMC)).

In one embodiment, an EphA2/EphrinA1 Modulator of the invention is administered to a subject in need thereof in combination with a therapy currently used or known to treat, manage, prevent and/or ameliorate cirrhosis and/or fibrosis (e.g., fibrosis of the liver, kidney, lungs, heart, retina and other viscera). In a specific embodiment, the non-neoplastic epithelial and/or endothelial cell disorder is lung fibrosis and the non-EphA2/EphrinA1 Modulator therapy is, e.g., recombinant human relaxin such as ConXn™, methylprednisolone, cyclophosphamid, corticosteroids, azathioprine, cyclophosphamide, penicillamine, colchicine, cyclosporine, prednisoline, pirfenidone, TGF-β inhibitors, INF-γ, TNF-α antagonists, antiangiogenic factors (e.g., IP-10), angiotensin-converting enzyme inhibitors, angiotensin II receptor antagonists, N-acetylcysteine, and/or endothelin receptor antagonists. In another embodiment, an EphA2/EphrinA1 Modulator of the invention is administered in combination with a therapy currently used or known to treat, manage, prevent and/or ameliorate asthma, ischemia, atherosclerosis, diabetic retinopathy, macular degeneration, rheumatoid arthritis, osteoarthritis and/or psoriasis. In another embodiment, an EphA2/EphrinA1 Modulator of the invention is administered to a subject in need thereof in combination with an immunomodulatory agent. In another embodiment, an EphA2/EphrinA1 Modulator of the invention is administered to a subject in need thereof in combination with an anti-inflammatory agent. In another embodiment, an EphA2/EphrinA1 Modulator of the invention is administered to a subject in need thereof in combination with an anti-angiogenic agent. In yet another embodiment, an EphA2/EphrinA1 Modulator of the invention is administered to a subject in need thereof in combination with a TNF-α antagonist.

The therapies can be administered to a subject in need thereof sequentially or concurrently. In particular, the therapies should be administered to a subject at exactly the same time or in a sequence within a time interval such that the therapies can act together to provide an increased benefit than if they were administered otherwise. In a specific embodiment, the combination therapies of the invention comprise an effective amount of one or more EphA2/EphrinA1 Modulators of the invention and an effective amount of at least one other therapy which has the same mechanism of action as said EphA2/EphrinA1 Modulators of the invention. In a specific embodiment, the combination therapies of the invention comprise an effective amount of one or more EphA2/EphrinA1 Modulators of the invention and an effective amount of at least one other therapy (e.g., prophylactic or therapeutic agent) which has a different mechanism of action than said EphA2/EphrinA1 Modulators of the invention. In certain embodiments, the combination therapies of the present invention improve the prophylactic or therapeutic effect of one or more antibodies of the invention by functioning together with the EphA2/EphrinA1 Modulators of the invention to have an additive or synergistic effect. In certain embodiments, the combination therapies of the present invention reduce the side effects associated with the prophylactic or therapeutic agents. In various embodiments, the therapies are administered to a patient less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, a about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more therapies are administered within the same patient visit.

The prophylactic or therapeutic agents of the combination therapies can be administered to a subject, preferably a human subject, in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

In a specific embodiment, a pharmaceutical composition comprising one or more EphA2/EphrinA1 Modulators of the invention described herein is administered to a subject, preferably a human, to prevent, treat, manage and/or ameliorate a non-neoplastic hyperproliferative epithelial and/or endothelial cell disorder or a symptom thereof. In accordance with the invention, pharmaceutical compositions of the invention may also comprise one or more therapies (e.g., prophylactic or therapeutic agents), other than the EphA2/EphrinA1 Modulators of the invention, which are currently being used, have been used, or are known to be useful in the prevention, treatment or amelioration of one or more symptoms associated with a non-neoplastic hyperproliferative epithelial and/or endothelial cell disorder.

4.5.2.1 Immunomodulatory Therapies

In certain embodiments, the present invention provides compositions comprising one or more EphA2/EphrinA1 Modulators of the invention and one or more immunomodulatory agents (i.e., agents which modulate the immune response in a subject), and methods for treating, managing or preventing a non-neoplastic hyperproliferative epithelial and/or endothelial cell disorder, (e.g., cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis) or a symptom thereof, in a subject comprising the administration of said compositions. The invention also provides methods for treating, managing or preventing a non-neoplastic hyperproliferative epithelial and/or endothelial cell disorder or a symptom thereof comprising the administration of an EphA2/EphrinA1 Modulator in combination with one or more immunomodulatory agents. In a specific embodiment of the invention, the immunomodulatory agent inhibits or suppresses the immune response in a human subject. Immunomodulatory agents are well-known to one skilled in the art and can be used in the methods and compositions of the invention.

Any immunomodulatory agent well-known to one of skill in the art may be used in the methods and compositions of the invention. Immunomodulatory agents can affect one or more or all aspects of the immune response in a subject. Aspects of the immune response include, but are not limited to, the inflammatory response, the complement cascade, leukocyte and lymphocyte differentiation, proliferation, and/or effector function, lymphocyte, monocyte and/or basophil counts, and the cellular communication among cells of the immune system. In certain embodiments of the invention, an immunomodulatory agent modulates one aspect of the immune response. In other embodiments, an immunomodulatory agent modulates more than one aspect of the immune response. In a preferred embodiment of the invention, the administration of an immunomodulatory agent to a subject inhibits or reduces one or more aspects of the subject's immune response capabilities. In a specific embodiment of the invention, the immunomodulatory agent inhibits or suppresses the immune response in a subject. In accordance with the invention, an immunomodulatory agent is not antibody that immunospecifically binds to an EphA2 or an EphrinA1 polypeptide. In certain embodiments, an immunomodulatory agent is not an anti-inflammatory agent. In certain embodiments, an immunomodulatory agent is not an anti-angiogenic agent. In other emobidments, an immunomodulatory agent is not a TNF α antagonist. In certain embodiments, an immunomodulatory agent is a chemotherapeutic agent. In certain embodiments, an immunomodulatory agent is not a chemotherapeutic agent.

Examples of immunomodulatory agents include, but are not limited to, proteinaceous agents such as cytokines, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules and triple helices), small molecules, organic compounds, and inorganic compounds. In particular, immunomodulatory agents include, but are not limited to, methotrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamides (e.g., leflunamide), T cell receptor modulators, cytokine receptor modulators, and modulators mast cell modulators.

In a specific embodiment, an immunomodulatory agent is a T cell receptor modulator. As used herein, the term “T cell receptor modulator” refers to an agent which modulates the phosphorylation of a T cell receptor, the activation of a signal transduction pathway associated with a T cell receptor and/or the expression of a particular protein associated with T cell receptor activity such as a cytokine. Such an agent may directly or indirectly modulate the phosphorylation of a T cell receptor, and/or the expression of a particular protein associated with T cell receptor activity such as a cytokine. Examples of T cell receptor modulators include, but are not limited to, anti-T cell receptor antibodies (e.g., anti-CD4 antibodies (e.g., cM-T412 (Boehringer), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or RITUXAN™ which is a chimeric anti-CD20 IgG1 antibody (IDEC Pharm/Genentech, Roche/Zettyaku), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies (e.g., siplizumab (Medimmune, Inc., International Publication Nos. WO 02/098370 and WO 02/069904)), anti-CD11a antibodies (e.g., Xanelim (Genentech)), and anti-B7 antibodies (e.g., IDEC-114) (IDEC))), CTLA4-immunoglobulin, and LFA-3TIP (Biogen, International Publication No. WO 93/08656 and U.S. Pat. No. 6,162,432). In a specific embodiment, a T cell receptor modulator is siplizumab (MedImmune, Inc., International Publication Nos. WO 02/098370 and WO 02/069904).

In a specific embodiment, an immunomodulatory agent is a cytokine receptor modulator. As used herein, the term “cytokine receptor modulator” refers to an agent which modulates the phosphorylation of a cytokine receptor, the activation of a signal transduction pathway associated with a cytokine receptor, and/or the expression of a particular protein such as a cytokine or cytokine receptor. Such an agent may directly or indirectly modulate the phosphorylation of a cytokine receptor, the activation of a signal transduction pathway associated with a cytokine receptor, and/or the expression of a particular protein such as a cytokine. Examples of cytokine receptor modulators include, but are not limited to, soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof), cytokines or fragments thereof (e.g., interleukin IL-2, IL-3, IL-4, IL-5, IL-6, IL-7, IL-8, IL-9, IL-10, IL-11, IL-12, IL-13, IL-15, IL-23, TNF-α, TNF-β, interferon (IFN)-α, IFN-β, IFN-γ, and GM-CSF), anti-cytokine receptor antibodies (e.g., anti-IFN receptor antibodies, anti-IL-2 receptor antibodies (e.g., Zenapax (Protein Design Labs)), anti-IL-3 receptor antibodies, anti-IL-4 receptor antibodies, anti-IL-6 receptor antibodies, anti-IL-9 receptor antibodies, anti-IL-10 receptor antibodies, anti-IL-12 receptor antibodies, anti-IL-13 receptor antibodies, anti-IL-15 receptor antibodies, and anti-IL-23 receptor antibodies), anti-cytokine antibodies (e.g., anti-IFN antibodies, anti-TNF-α antibodies, anti-IL-1β antibodies, anti-IL-3 antibodies, anti-IL-6 antibodies, anti-IL-8 antibodies (e.g., ABX-IL-8 (Abgenix)), anti-IL-9 antibodies, anti-IL-12 antibodies, anti-IL-13 antibodies, anti-IL-15 antibodies, and anti-IL-23 antibodies).

In a specific embodiment, a cytokine receptor modulator is IL-3, IL-4, IL-10, or a fragment thereof. In another embodiment, a cytokine receptor modulator is an anti-IL-1β antibody, anti-IL-6 antibody, anti-IL-12 receptor antibody, or anti-TNF-α antibody. In another embodiment, a cytokine receptor modulator is the extracellular domain of a TNF-α receptor or a fragment thereof. In certain embodiments, a cytokine receptor modulator is not a TNF-α antagonist.

In a preferred embodiment, the immunomodulatory agent decreases the amount of IL-9. In a more preferred embodiment, the immunomodulatory agent is an antibody (preferably a monoclonal antibody) or fragment thereof that immunospecifically binds to IL-9 (see, e.g., U.S. patent application Ser. No. 10/823,810, filed Apr. 12, 2004 entitled “Methods of Preventing or Treating Respiratory Conditions” by Reed (Attorney Docket No. 10271-113-999), U.S. patent application Ser. No. 10/823,523 filed Apr. 12, 2004 entitled “Recombinant IL-9 Antibodies and Uses Thereof” by Reed (Attorney Docket No. 10271-112-999), and U.S. Provisional Application No. 60/561,845 filed Apr. 12, 2004 entitled “Anti-IL-9 Antibody Formulations and Uses Thereof” by Reed (Attorney Docket No. 10271-126-888), all of which are incorporated by reference herein in their entireties. Although not intending to be bound by a particular mechanism of action, the use of anti-IL-9 antibodies neutralize the ability of IL-9 to have a biological effect and thereby blocks or decreases inflammatory cell recruitment.

In one embodiment, a cytokine receptor modulator is a mast cell modulator. In an alternative embodiment, a cytokine receptor modulator is not a mast cell modulator. Examples of mast cell modulators include, but are not limited to stem cell factor (c-kit receptor ligand) inhibitors (e.g., mAb 7H6, mAb 8H7a, pAb 1337, FK506, CsA, dexamthasone, and fluconcinonide), c-kit receptor inhibitors (e.g., STI 571 (formerly known as CGP 57148B)), mast cell protease inhibitors (e.g., GW-45, GW-58, wortmannin, LY 294002, calphostin C, cytochalasin D, genistein, KT5926, staurosproine, and lactoferrin), relaxin (“RLX”), IgE antagonists (e.g., antibodies rhuMAb-E25 omalizumab, HMK-12 and 6HD5, and mAB Hu-901), IL-3 antagonists, IL-4 antagonists, IL-10 antagonists, and TGF-beta.

An immunomodulatory agent may be selected to interfere with binding and/or activation of B cell markers and/or receptors. In a specific embodiment, the immunomodulatory agent is an antibody that binds to a B cell marker and/or a receptor.

An immunomodulatory agent may be selected to interfere with the interactions between the T helper subsets (TH1 or TH2) and B cells to inhibit neutralizing antibody formation. Antibodies that interfere with or block the interactions necessary for the activation of B cells by TH (T helper) cells, and thus block the production of neutralizing antibodies, are useful as immunomodulatory agents in the methods of the invention. For example, B cell activation by T cells requires certain interactions to occur (Durie et al., Immunol. Today, 15(9):406-410 (1994)), such as the binding of CD40 ligand on the T helper cell to the CD40 antigen on the B cell, and the binding of the CD28 and/or CTLA4 ligands on the T cell to the B7 antigen on the B cell. Without both interactions, the B cell cannot be activated to induce production of the neutralizing antibody.

The CD40 ligand (CD40L)-CD40 interaction is a desirable point to block the immune response because of its broad activity in both T helper cell activation and function as well as the absence of redundancy in its signaling pathway. Thus, in a specific embodiment of the invention, the interaction of CD40L with CD40 is transiently blocked at the time of administration of one or more of the immunomodulatory agents. This can be accomplished by treating with an agent which blocks the CD40 ligand on the TH cell and interferes with the normal binding of CD40 ligand on the T helper cell with the CD40 antigen on the B cell. An antibody to CD40 ligand (anti-CD40L) (available from Bristol-Myers Squibb Co; see, e.g., European patent application 555,880, published Aug. 18, 1993) or a soluble CD40 molecule can be selected and used as an immunomodulatory agent in accordance with the methods of the invention.

An immunomodulatory agent may be selected to inhibit the interaction between TH1 cells and cytotoxic T lymphocytes (“CTLs”) to reduce the occurrence of CTL-mediated killing. An immunomodulatory agent may be selected to alter (e.g., inhibit or suppress) the proliferation, differentiation, activity and/or function of the CD4⁺ and/or CD8⁺ T cells. For example, antibodies specific for T cells can be used as immunomodulatory agents to deplete, or alter the proliferation, differentiation, activity and/or function of CD4⁺ and/or CD8⁺ T cells.

In one embodiment of the invention, an immunomodulatory agent that reduces or depletes T cells, preferably memory T cells, is administered to a subject at risk of or with a disease or disorder associated with or characterized by aberrant expression and/or activity of an IL-9 polypeptide, a disease or disorder associated with or characterized by aberrant expression of an IL-9R or one or more subunits thereof, an autoimmune disease, an inflammatory disease, a proliferative disease, or an infection (preferably, a respiratory infection) in accordance with the methods of the invention. See, e.g., U.S. Pat. No. 4,658,019. In another embodiment of the invention, an immunomodulatory agent that inactivates CD8⁺ T cells is administered to a subject at risk of or with non-neoplastic hyperproliferative epithelial and/or endothelial cell disorder in accordance with the methods of the invention. In a specific embodiment, anti-CD8 antibodies are used to reduce or deplete CD8⁺ T cells.

In another embodiment, an immunomodulatory agent which reduces or inhibits one or more biological activities (e.g., the differentiation, proliferation, and/or effector functions) of TH0, TH1, and/or TH2 subsets of CD4⁺ T helper cells is administered to a subject at risk of or with a non-neoplastic hyperproliferative epithelial and/or endothelial cell disorder in accordance with the methods of the invention. One example of such an immunomodulatory agent is IL-4. IL-4 enhances antigen-specific activity of TH2 cells at the expense of the TH1 cell function (see, e.g., Yokota et al, 1986 Proc. Natl. Acad. Sci., USA, 83:5894-5898; and U.S. Pat. No. 5,017,691). Other examples of immunomodulatory agents that affect the biological activity (e.g., proliferation, differentiation, and/or effector functions) of T-helper cells (in particular, TH1 and/or TH2 cells) include, but are not limited to, IL-2, IL-4, IL-5, IL-6, IL-10, IL-12, IL-13, IL-15, IL-23, and interferon (IFN)-γ.

In another embodiment, an immunomodulatory agent administered to a subject at risk of or with a non-neoplastic hyperproliferative epithelial and/or endothelial cell disorder in accordance with the methods of the invention is a cytokine that prevents antigen presentation. In a specific embodiment, an immunomodulatory agent used in the methods of the invention is IL-10. IL-10 also reduces or inhibits macrophage action which involves bacterial elimination.

An immunomodulatory agent may be selected to reduce or inhibit the activation, degranulation, proliferation, and/or infiltration of mast cells. In certain embodiments, the immunomodulatory agent interferes with the interactions between mast cells and mast cell activating agents, including, but not limited to stem cell factors (c-kit ligands), IgE, IL-4, environmental irritants, and infectious agents. In a specific embodiment, the immunomodulatory agent reduces or inhibits the response of mast cells to environmental irritants such as, but not limited to pollen, dust mites, tobacco smoke, and/or pet dander. In another specific embodiment, the immunomodulatory agent reduces or inhibits the response of mast cells to infectious agents, such as viruses, bacteria, and fungi. Examples of mast cell modulators that reduce or inhibit the activation, degranulation, proliferation, and/or infiltration of mast cells include, but are not limited to, stem cell factor (c-kit receptor ligand) inhibitors (e.g., mAb 7H6, mAb 8H7a, and pAb 1337 (see Mendiaz et al., 1996, Eur J Biochem 293(3):842-849), FK506 and CsA (Ito et al., 1999 Arch Dermatol Res 291(5):275-283), dexamthasone and fluconcinonide (see Finooto et al., 1997, J. Clin. Invest. 99(7):1721-1728)), c-kit receptor inhibitors (e.g., STI 571 (formerly known as CGP 57148B) (see Heinrich et al., 2000 Blood 96(3):925-932)), mast cell protease inhibitors (e.g., GW-45 and GW-58 (see, Temkin et al., 2002, J Immunol 169(5):2662-2669), wortmannin, LY 294002, calphostin C, and cytochalasin D (see Vosseller et al., 1997, Mol Biol Cell 1997:909-922), genistein, KT5926, and staurosproine (see Nagai et al. 1995, Biochem Biophys Res Commun 208(2):576-581), and lactoferrin (see He et al., 2003 Biochem Pharmacol 65(6):1007-1015)), relaxin (“RLX”) (see Bani et al., 2002 Int Immunopharmacol 2(8):1195-1294), ), IgE antagonists (e.g., antibodies rhuMAb-E25 omalizumab (see Finn et al., 2003 J Allergy Clin Immuno 111(2):278-284; Corren et al., 2003 J Allergy Clin Immuno 111(1):87-90; Busse and Neaville, 2001 Curr Opin Allergy Clin Immunol. 1(1):105-108; and Tang and Powell, 2001, Eur J Pediatr 160(12): 696-704), HMK-12 and 6HD5 (see Miyajima et al., 2202 Int Arch Allergy Immuno 128(1):24-32), and mAB Hu-901 (see van Neerven et al., 2001 Int Arch Allergy Immuno 124(1-3):400), IL-3 antagonist, IL-4 antagonists, IL-10 antagonists, and TGF-beta (see Metcalfe et al., 1995, Exp Dermatol 4(4 Pt 2):227-230).

In a preferred embodiment, proteins, polypeptides or peptides (including antibodies) that are utilized as immunomodulatory agents are derived from the same species as the recipient of the proteins, polypeptides or peptides so as to reduce the likelihood of an immune response to those proteins, polypeptides or peptides. In another preferred embodiment, when the subject is a human, the proteins, polypeptides, or peptides that are utilized as immunomodulatory agents are human or humanized. The immunomodulator activity of an immunomodulatory agent can be determined by CTL assays, proliferation assays, immunoassays (e.g. ELISAs) for the expression of particular proteins such as co-stimulatory molecules and cytokines, and FACS.

In accordance with the invention, one or more immunomodulatory agents are administered to a subject at risk of or with a non-neoplastic hyperproliferative epithelial and/or endothelial cell disorder prior to, subsequent to, or concomitantly with an antibody that immunospecifically binds to an EphA2 or EphrinA1 polypeptide. Preferably, one or more immunomodulatory agents are administered in combination with an antibody that immunospecifically binds to an EphA2 or EphrinA1 polypeptide to a subject at risk of or with a non-neoplastic hyperproliferative epithelial and/or endothelial cell disorder to reduce or inhibit one or more aspects of the immune response as deemed necessary by one of skill in the art. Any technique well-known to one skilled in the art can be used to measure one or more aspects of the immune response in a particular subject, and thereby determine when it is necessary to administer an immunomodulatory agent to said subject. In a preferred embodiment, a mean absolute lymphocyte count of approximately 500 cells/mm³, preferably 600 cells/mm³, 650 cells/mm³, 700 cells/mm³, 750 cells/mm³, 800 cells/mm³, 900 cells/mm³, 1000 cells/mm³, 1100 cells/mm³, or 1200 cells/mm³ is maintained in a subject. In another preferred embodiment, a subject at risk of or with a non-neoplastic hyperproliferative epithelial and/or endothelial cell disorder is not administered an immunomodulatory agent if their absolute lymphocyte count is 500 cells/mm³ or less, 550 cells/mm³ or less, 600 cells/mm³ or less, 650 cells/mm³ or less, 700 cells/mm³ or less, 750 cells/mm³ or less, or 800 cells/mm³ or less.

In a preferred embodiment, one or more immunomodulatory agents are administered in combination with an antibody that immunospecifically binds to an EphA2 or EphrinA1 polypeptide to a subject at risk of or with a non-neoplastic hyperproliferative epithelial and/or endothelial cell disorder so as to transiently reduce or inhibit one or more aspects of the immune response. Such a transient inhibition or reduction of one or more aspects of the immune system can last for hours, days, weeks, or months. Preferably, the transient inhibition or reduction in one or more aspects of the immune response lasts for a few hours (e.g., 2 hours, 4 hours, 6 hours, 8 hours, 12 hours, 14 hours, 16 hours, 18 hours, 24 hours, 36 hours, or 48 hours), a few days (e.g., 3 days, 4 days, 5 days, 6 days, 7 days, or 14 days), or a few weeks (e.g., 3 weeks, 4 weeks, 5 weeks or 6 weeks). The transient reduction or inhibition of one or more aspects of the immune response enhances the prophylactic and/or therapeutic effect(s) of EphA2/EphrinA1 Modulator.

Nucleic acid molecules encoding proteins, polypeptides, or peptides with immunomodulatory activity or proteins, polypeptides, or peptides with immunomodulatory activity can be administered to a subject at risk of or with a non-neoplastic hyperproliferative epithelial and/or endothelial cell disorder in accordance with the methods of the invention. Further, nucleic acid molecules encoding derivatives, analogs, or fragments of proteins, polypeptides, or peptides with immunomodulatory activity, or derivatives, analogs, or fragments of proteins, polypeptides, or peptides with immunomodulatory activity can be administered to a subject at risk of or with a non-neoplastic hyperproliferative epithelial and/or endothelial cell disorder in accordance with the methods of the invention. Preferably, such derivatives, analogs, and fragments retain the immunomodulatory activity of the full-length, wild-type protein, polypeptide, or peptide.

4.5.2.2 Anti-Inflammatory Therapies

Any anti-inflammatory agent, including agents useful in therapies for inflammatory disorders, well-known to one of skill in the art can be used in the compositions and methods of the invention. Non-limiting examples of anti-inflammatory agents include non-steroidal anti-inflammatory drugs (NSAIDs), steroidal anti-inflammatory drugs, anticholinergics (e.g., atropine sulfate, atropine methylnitrate, and ipratropium bromide (ATROVENT™)), beta2-agonists (e.g., abuterol (VENTOLIN™ and PROVENTIL™), bitolterol (TORNALATE™), levalbuterol (XOPONEX™), metaproterenol (ALUPEN™), pirbuterol (MAXAIR™), terbutlaine (BRETHAIRE™ and BRETHINE™), albuterol (PROVENTIL™, REPETABS™, and VOLMAX™), formoterol (FORADIL AEROLIZER™), and salmeterol (SEREVENT™ and SEREVENT DISKUS™)), and methylxanthines (e.g., theophylline (UNIPHYL™, THEO-DUR™, SLO-BID™, AND TEHO-42™)). Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxgenase enzyme (e.g., COX-1 and/or COX-2). Examples of steroidal anti-inflammatory drugs include, but are not limited to, glucocorticoids, dexamethasone (DECADRON™), corticosteroids (e.g., methylprednisolone (MEDROL™)), cortisone, hydrocortisone, prednisone (PREDNISONE™ and DELTASONE™), prednisolone (PRELONE™ and PEDIAPRED™), triamcinolone, azulfidine, and inhibitors of eicosanoids (e.g., prostaglandins, thromboxanes, and leukotrienes (see Table 6, infra, for non-limiting examples of leukotriene and typical dosages of such agents)).

In certain embodiments, the anti-inflammatory agent is an agent useful in the prevention, management, treatment, and/or amelioration of asthma or one or more symptoms thereof. Non-limiting examples of such agents include adrenergic stimulants (e.g., catecholamines (e.g., epinephrine, isoproterenol, and isoetharine), resorcinols (e.g., metaproterenol, terbutaline, and fenoterol), and saligenins (e.g., salbutamol)), adrenocorticoids, blucocorticoids, corticosteroids (e.g., beclomethadonse, budesonide, flunisolide, fluticasone, triamcinolone, methylprednisolone, prednisolone, and prednisone), other steroids, beta2-agonists (e.g., albtuerol, bitolterol, fenoterol, isoetharine, metaproterenol, pirbuterol, salbutamol, terbutaline, formoterol, salmeterol, and albutamol terbutaline), anti-cholinergics (e.g., ipratropium bromide and oxitropium bromide), IL-4 antagonists (including antibodies), IL-5 antagonists (including antibodies), IL-13 antagonists (including antibodies), PDE4-inhibitor, NF-Kappa-β inhibitor, VLA-4 inhibitor, CpG, anti-CD23, selectin antagonists (TBC 1269), mast cell protease inhibitors (e.g., tryptase kinase inhibitors (e.g., GW-45, GW-58, and genisteine), phosphatidylinositide-3′ (PI3)-kinase inhibitors (e.g., calphostin C), and other kinase inhibitors (e.g., staurosporine) (see Temkin et al., 2002 J Immunol 169(5):2662-2669; Vosseller et al., 1997 Mol. Biol. Cell 8(5):909-922; and Nagai et al., 1995 Biochem Biophys Res Commun 208(2):576-581)), a C3 receptor antagonists (including antibodies), immunosuppressant agents (e.g., methotrexate and gold salts), mast cell modulators (e.g., cromolyn sodium (INTAL™) and nedocromil sodium (TILADE™)), and mucolytic agents (e.g., acetylcysteine)). In a specific embodiment, the anti-inflammatory agent is a leukotriene inhibitor (e.g., montelukast (SINGULAIR™), zafirlukast (ACCOLAT™), pranlukast (ONON™), or zileuton (ZYFLO™) (see Table 6)).

TABLE 6
Leukotriene Inhibitors for Asthma Therapy

| Leukotriene Modifier | Usual Daily Dosage |
|---|---|
| Montelukast | 4 mg for 2-5 years old |
| (SINGULAIR ™) | 5 mg for 6 to 15 years old |
| | 10 mg for 15 years and older |
| Zafirlukast | 10 mg b.i.d. for 5 to 12 years old twice daily |
| (ACCOLATE ™) | 20 mg b.i.d. for 12 years or older twice daily |
| Pranlukast (ONON ™) | Only avialable in Asia |
| Zyleuton (ZYFLO ™) | 600 mg four times a day for 12 years and |
| | older |

In certain embodiments, the anti-inflammatory agent is an agent useful in preventing, treating, managing, and/or ameliorating allergies or one or more symptoms thereof. Non-limiting examples of such agents include antimediator drugs (e.g., antihistamine, see Table 7, infra for non-limiting examples of antihistamine and typical dosages of such agents), corticosteroids, decongestants, sympathomimetic drugs (e.g., α-adrenergic and β-adrenergic drugs), TNX901 (Leung et al., 2003, N Engl J Med 348(11):986-993), IgE antagonists (e.g., antibodies rhuMAb-E25 omalizumab (see Finn et al., 2003 J Allergy Clin Immuno 111(2):278-284; Corren et al., 2003 J Allergy Clin Immuno 111(1):87-90; Busse and Neaville, 2001 Curr Opin Allergy Clin Immuno 1(1): 105-108; and Tang and Powell, 2001, Eur J Pediatr 160(12): 696-704), HMK-12 and 6HD5 (see Miyajima et al., 2202 Int Arch Allergy Immuno 128(1):24-32), and mAB Hu-901 (see van Neerven et al., 2001 Int Arch Allergy Immuno 124(1-3):400), theophylline and its derivatives, glucocorticoids, and immunotherapies (e.g., repeated long-term injection of allergen, short course desensitization, and venom immunotherapy).

TABLE 7
H₁ Antihistamines

| Chemical class and | |
|---|---|
| representative drugs | Usual daily dosage |
| Ethanolamine | |
| Diphehydramine | 25-50 mg every 4-6 hours |
| Clemastine | 0.34-2.68 mg every 12 hours |
| Ethylenediamine | |
| Tripelennamine | 25-50 mg every 4-6 hours |
| Alkylamine | |
| Brompheniramine | 4 mg every 4-6 hours; or 8-12 mg of SR |
| | form every 8-12 hour |
| Chlorpheniramine | 4 mg every 4-6 hours; or 8-12 mg of SR |
| | form every 8-12 hour |
| Triprolidine (1.25 mg/5 ml) | 2.5 mg every 4-6 hours |
| Phenothiazine | |
| Promethazine | 25 mg at bedtime |
| Piperazine | |
| Hydroxyzine | 25 mg every 6-8 hours |
| Piperidines | |
| Astemizole (nonsedating) | 10 mg/day |
| Azatadine | 1-2 mg every 12 hours |
| Cetirzine | 10 mg/day |
| Cyproheptadine | 4 mg every 6-8 hour |
| Fexofenadine (nonsedating) | 60 mg every 12 hours |
| Loratidine (nonsedating) | 10 mg every 24 hours |

Anti-inflammatory therapies and their dosages, routes of administration, and recommended usage are known in the art and have been described in such literature as the Physician's Desk Reference (58th ed., 2004).

4.5.2.3 Anti-Angiogenic Therapies

Any anti-angiogenic agent well-known to one of skill in the art can be used in the compositions and methods of the invention. Non-limiting examples of anti-angiogenic agents include proteins, polypeptides, peptides, fusion proteins, antibodies (e.g., human, humanized, chimeric, monoclonal, polyclonal, Fvs, ScFvs, Fab fragments, F(ab)₂ fragments, and antigen-binding fragments thereof) such as antibodies that immunospecifically bind to TNF-α, nucleic acid molecules (e.g., antisense molecules or triple helices), organic molecules, inorganic molecules, and small molecules that reduce or inhibit angiogenesis. In particular, examples of anti-angiogenic agents, include, but are not limited to, endostatin, angiostatin, apomigren, anti-angiogenic antithrombin III, the 29 kDa N-terminal and a 40 kDa C-terminal proteolytic fragments of fibronectin, a uPA receptor antagonist, the 16 kDa proteolytic fragment of prolactin, the 7.8 kDa proteolytic fragment of platelet factor-4, the anti-angiogenic 24 amino acid fragment of platelet factor-4, the anti-angiogenic factor designated 13.40, the anti-angiogenic 22 amino acid peptide fragment of thrombospondin I, the anti-angiogenic 20 amino acid peptide fragment of SPARC, RGD and NGR containing peptides, the small anti-angiogenic peptides of laminin, fibronectin, procollagen and EGF, integrin αᵥβ₃ antagonists, acid fibroblast growth factor (aFGF) antagonists, basic fibroblast growth factor (bFGF) antagonists, vascular endothelial growth factor (VEGF) antagonists (e.g., anti-VEGF antibodies (e.g., AVASTIN™ (Genentech)), VEGF receptor (VEGFR) antagonists (e.g., anti-VEGFR antibodies) and anti-integrin antagonists (e.g., REOPRO® (abciximab) (Centocor) which binds to the glycoprotein IIb/IIia receptor on the platelets for the prevention of clot formation).

Examples of integrin αᵥβ₃ antagonists include, but are not limited to, proteinaceous agents such as non-catalytic metalloproteinase fragments, RGD peptides, peptide mimetics, fusion proteins, disintegrins or derivatives or analogs thereof, and antibodies that immunospecifically bind to integrin αᵥβ₃, nucleic acid molecules, organic molecules, and inorganic molecules. Non-limiting examples of antibodies that immunospecifically bind to integrin αᵥβ₃ include 11D2 (Searle), LM609 (Scripps), and VITAXIN™ (MedImmune, Inc.). Non-limiting examples of small molecule peptidometric integrin αᵥβ₃ antagonists include S836 (Searle) and S448 (Searle). Examples of disintegrins include, but are not limited to, Accutin. The invention also encompasses the use of any of the integrin αᵥβ₃ antagonists disclosed in the following U.S. Patents and International publications in the compositions and methods of the invention: U.S. Pat. Nos. 5,149,780; 5,196,511; 5,204,445; 5,262,520; 5,306,620; 5,478,725; 5,498,694; 5,523,209; 5,578,704; 5,589,570; 5,652,109; 5,652,110; 5,693,612; 5,705,481; 5,753,230; 5,767,071; 5,770,565; 5,780,426; 5,817,457; 5,830,678; 5,849,692; 5,955,572; 5,985,278; 6,048,861; 6,090,944; 6,096,707; 6,130,231; 6,153,628; 6,160,099; and 6,171,588; and International Publication Nos. WO 95/22543; WO 98/33919; WO 00/78815; and WO 02/070007, each of which is incorporated herein by reference in its entirety. In a preferred embodiment, the anti-angiogenic agent is VITAXIN™ (MedImmune, Inc.) or an antigen-binding fragment thereof.

In a specific embodiment of the invention, an anti-angiogenic agent is endostatin. Naturally occurring endostatin consists of the C-terminal 180 amino acids of collagen XVIII (cDNAs encoding two splice forms of collagen XVIII have GenBank Accession Nos. AF18081 and AF18082). In another embodiment of the invention, an anti-angiogenic agent is a plasminogen fragment (the coding sequence for plasminogen can be found in GenBank Accession Nos. NM_{—}000301 and A33096). Angiostatin peptides naturally include the four kringle domains of plasminogen, kringle 1 through kringle 4. It has been demonstrated that recombinant kringle 1, 2 and 3 possess the anti-angiogenic properties of the native peptide, whereas kringle 4 has no such activity (Cao et al., 1996, J. Biol. Chem. 271:29461-29467). Accordingly, the angiostatin peptides comprises at least one and preferably more than one kringle domain selected from the group consisting of kringle 1, kringle 2 and kringle 3. In a specific embodiment, the anti-angiogenic peptide is the 40 kDa isoform of the human angiostatin molecule, the 42 kDa isoform of the human angiostatin molecule, the 45 kDa isoform of the human angiostatin molecule, or a combination thereof. In another embodiment, an anti-angiogenic agent is the kringle 5 domain of plasminogen, which is a more potent inhibitor of angiogenesis than angiostatin (angiostatin comprises kringle domains 1-4). In another embodiment of the invention, an anti-angiogenic agent is antithrombin III. Antithrombin III, which is referred to hereinafter as antithrombin, comprises a heparin binding domain that tethers the protein to the vasculature walls, and an active site loop which interacts with thrombin. When antithrombin is tethered to heparin, the protein elicits a conformational change that allows the active loop to interact with thrombin, resulting in the proteolytic cleavage of said loop by thrombin. The proteolytic cleavage event results in another change of conformation of antithrombin, which (i) alters the interaction interface between thrombin and antithrombin and (ii) releases the complex from heparin (Carrell, 1999, Science 285:1861-1862, and references therein). O'Reilly et al. (1999, Science 285:1926-1928) have discovered that the cleaved antithrombin has potent anti-angiogenic activity. Accordingly, in one embodiment, an anti-angiogenic agent is the anti-angiogenic form of antithrombin. In another embodiment of the invention, an anti-angiogenic agent is the 40 kDa and/or 29 kDa proteolytic fragment of fibronectin.

In another embodiment of the invention, an anti-angiogenic agent is a urokinase plasminogen activator (uPA) receptor antagonist. In one mode of the embodiment, the antagonist is a dominant negative mutant of uPA (see, e.g., Crowley et al., 1993, Proc. Natl. Acad. Sci. USA 90:5021-5025). In another mode of the embodiment, the antagonist is a peptide antagonist or a fusion protein thereof (Goodson et al., 1994, Proc. Natl. Acad. Sci. USA 91:7129-7133). In yet another mode of the embodiment, the antagonist is a dominant negative soluble uPA receptor (Min et al., 1996, Cancer Res. 56:2428-2433). In another embodiment of the invention, a therapeutic molecule of the invention is the 16 kDa N-terminal fragment of prolactin, comprising approximately 120 amino acids, or a biologically active fragment thereof (the coding sequence for prolactin can be found in GenBank Accession No. NM_{—}000948). In another embodiment of the invention, an anti-angiogenic agent is the 7.8 kDa platelet factor-4 fragment. In another embodiment of the invention, a therapeutic molecule of the invention is a small peptide corresponding to the anti-angiogenic 13 amino acid fragment of platelet factor-4, the anti-angiogenic factor designated 13.40, the anti-angiogenic 22 amino acid peptide fragment of thrombospondin I, the anti-angiogenic 20 amino acid peptide fragment of SPARC, the small anti-angiogenic peptides of laminin, fibronectin, procollagen, or EGF, or small peptide antagonists of integrin α_{c}β₃ or the VEGF receptor. In another embodiment, the small peptide comprises an RGD or NGR motif. In certain embodiments, an anti-angiogenic agent is a TNF-α antagonist. In other embodiments, an anti-angiogenic agent is not a TNF-α antagonist.

Nucleic acid molecules encoding proteins, polypeptides, or peptides with anti-angiogenic activity, or proteins, polypeptides or peptides with anti-angiogenic activity can be administered to a subject at risk of or with a non-neoplastic hyperproliferative epithelial and/or endothelial cell disorder in accordance with the methods of the invention. Further, nucleic acid molecules encoding derivatives, analogs, fragments, or variants of proteins, polypeptides, or peptides with anti-angiogenic activity, or derivatives, analogs, fragments, or variants of proteins, polypeptides, or peptides with anti-angiogenic activity can be administered to a subject at risk of or with a non-neoplastic hyperproliferative epithelial and/or endothelial cell disorder in accordance with the methods of the invention. Preferably, such derivatives, analogs, variants, and fragments retain the anti-angiogenic activity of the full-length, wild-type protein, polypeptide, or peptide.

Proteins, polypeptides, or peptides that can be used as anti-angiogenic agents can be produced by any technique well-known in the art or described herein. Proteins, polypeptides or peptides with anti-angiogenic activity can be engineered so as to increase the in vivo half-life of such proteins, polypeptides, or peptides utilizing techniques well-known in the art or described herein. Preferably, anti-angiogenic agents that are commercially available are used in the compositions and methods of the invention. The anti-angiogenic activity of an agent can be determined in vitro and/or in vivo by any technique well-known to one skilled in the art.

4.5.2.4 TNF-α Antagonists

Any TNF-α antagonist well-known to one of skill in the art can be used in the compositions and methods of the invention. Non-limiting examples of TNF-α antagonists include proteins, polypeptides, peptides, fusion proteins, antibodies (e.g., human, humanized, chimeric, monoclonal, polyclonal, Fvs, ScFvs, Fab fragments, F(ab)₂ fragments, and antigen-binding fragments thereof) such as antibodies that immunospecifically bind to TNF-α, nucleic acid molecules (e.g., antisense molecules or triple helices), organic molecules, inorganic molecules, and small molecules that blocks, reduces, inhibits or neutralizes a function, an activity and/or expression of TNF-α. In various embodiments, a TNF-α antagonist reduces the function, activity and/or expression of TNF-α by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS) in an assay known to one of skill in the art.

Examples of antibodies that immunospecifically bind to TNF-α include, but are not limited to, infliximab (REMICADE®; Centacor), D2E7 (Abbott Laboratories/Knoll Pharmaceuticals Co., Mt. Olive, N.J.), CDP571 which is also known as HUMICADE™ and CDP-870 (both of Celltech/Pharmacia, Slough, U.K.), and TN3-19.12 (Williams et al., 1994, Proc. Natl. Acad. Sci. USA 91: 2762-2766; Thorbecke et al., 1992, Proc. Natl. Acad. Sci. USA 89:7375-7379). The present invention also encompasses the use of antibodies that immunospecifically bind to TNF-α disclosed in the following U.S. Patents in the compositions and methods of the invention: U.S. Pat. Nos. 5,136,021; 5,147,638; 5,223,395; 5,231,024; 5,334,380; 5,360,716; 5,426,181; 5,436,154; 5,610,279; 5,644,034; 5,656,272; 5,658,746; 5,698,195; 5,736,138; 5,741,488; 5,808,029; 5,919,452; 5,958,412; 5,959,087; 5,968,741; 5,994,510; 6,036,978; 6,114,517; and 6,171,787; each of which are herein incorporated by reference in their entirety. Examples of soluble TNF-α receptors include, but are not limited to, sTNF-R1 (Amgen), etanercept (ENBREL™; Immunex) and its rat homolog RENBREL™, soluble inhibitors of TNF-α derived from TNFrI, TNFrII (Kohno et al., 1990, Proc. Natl. Acad. Sci. USA 87:8331-8335), and TNF-α Inh (Seckinger et al, 1990, Proc. Natl. Acad. Sci. USA 87:5188-5192).

In one embodiment, a TNF-α antagonist used in the compositions and methods of the invention is a soluble TNF-α receptor. In a specific embodiment, a TNF-α antagonist used in the compositions and methods of the invention is etanercept (ENBREL™; Immunex) or a fragment, derivative or analog thereof. In another embodiment, a TNF-α antagonist used in the compositions and methods of the invention is an antibody that immunospecifically binds to TNF-α. In a specific embodiment, a TNF-α antagonist used in the compositions and methods of the invention is infliximab (REMICADE®; Centacor) a derivative, analog or antigen-binding fragment thereof.

Other TNF-α antagonists encompassed by the invention include, but are not limited to, IL-10, which is known to block TNF-α production via interferon γ-activated macrophages (Oswald et al. 1992, Proc. Natl. Acad. Sci. USA 89:8676-8680), TNFR-IgG (Ashkenazi et al., 1991, Proc. Natl. Acad. Sci. USA 88:10535-10539), the murine product TBP-1 (Serono/Yeda), the vaccine CytoTAb (Protherics), antisense molecule104838 (ISIS), the peptide RDP-58 (SangStat), thalidomide (Celgene), CDC-801 (Celgene), DPC-333 (Dupont), VX-745 (Vertex), AGIX-4207 (AtheroGenics), ITF-2357 (Italfarmaco), NPI-13021-31 (Nereus), SCIO-469 (Scios), TACE targeter (Immunix/AHP), CLX-120500 (Calyx), Thiazolopyrim (Dynavax), auranofin (Ridaura) (SmithKline Beecham Pharmaceuticals), quinacrine (mepacrine dichlorohydrate), tenidap (Enablex), Melanin (Large Scale Biological), and anti-p38 MAPK agents by Uriach.

Nucleic acid molecules encoding proteins, polypeptides, or peptides with TNF-α antagonist activity, or proteins, polypeptides, or peptides with TNF-α antagonist activity can be administered to a subject at risk of or with an inflammatory or autoimmune disease in accordance with the methods of the invention. Further, nucleic acid molecules encoding derivatives, analogs, fragments or variants of proteins, polypeptides, or peptides with TNF-α antagonist activity, or derivatives, analogs, fragments or variants of proteins, polypeptides, or peptides with TNF-α antagonist activity can be administered to a subject at risk of or with an inflammatory or autoimmune disease in accordance with the methods of the invention. Preferably, such derivatives, analogs, variants and fragments retain the TNF-α antagonist activity of the full-length, wild-type protein, polypeptide, or peptide.

Proteins, polypeptides, or peptides that can be used as TNF-α antagonists can be produced by any technique well-known in the art or described herein. Proteins, polypeptides or peptides with TNF-α antagonist activity can be engineered so as to increase the in vivo half-life of such proteins, polypeptides, or peptides utilizing techniques well-known in the art or described herein. Preferably, agents that are commercially available and known to function as TNF-α antagonists are used in the compositions and methods of the invention. The TNF-α antagonist activity of an agent can be determined in vitro and/or in vivo by any technique well-known to one skilled in the art.

4.6 Identification of EphA2/EphrinA1 Modulators of the Invention

The invention provides methods of assaying and screening for EphA2/EphrinA1 Modulators of the invention by incubating agents with cells that express EphA2 or EphrinA1, particularly epithelial and/or endothelial cells, and then assaying for an ability to modulate EphA2 and/or EphrinA1 gene expression and/or activities of EphA2 and/or EphrinA1 relative to a control (e.g., PBS or IgG), thereby identifying an EphA2/EphrinA1 Modulator of the invention. The invention also encompasses the use of in vivo assays to identify EphA2/EphrinA1 Modulator s, e.g., by reduction in symptoms (including pathological symptoms) in animal models of non-neoplastic hyperproliferative epithelial and/or endothelial cell disorders, such as cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis.

4.6.1 EphA2/EphrinA1 Modulators that Decrease EphA2 Cytoplasmic Tail Phosphorylation

The invention provides methods of assaying and screening for EphA2/EphrinA1 Modulators that decrease EphA2 cytoplasmic tail phosphorylation. Such EphA2/EphrinA1 Modulators of the invention decrease EphA2 internalization and degradation due to EphA2 cytoplasmic tail phosphorylation. Thus, EphA2 protein levels remain higher than they would otherwise in the absence of an EphA2/EphrinA1 Modulator that decreases EphA2 cytoplasmic tail phosphorylation. In one embodiment, EphA2/EphrinA1 Modulators decrease EphA2 cytoplasmic tail phosphorylation. In another embodiment, EphA2/EphrinA1 Modulators decrease Eph A2 internalization and degradation. Any method known in the art to assay either the level of EphA2 phosphorylation or expression can be used to screen EphA2/EphrinA1 Modulators to determine their ability to decrease EphA2 cytoplasmic tail phosphorylation or EphA2 degradation, e.g., immunoprecipitation, western blot, ELISAs, and phosphorylation assays (e.g., OMNI-PHOS™ kit available from Chemicon International, Temecula, Calif.). Ligand-mediated EphA2 cytoplasmic tail phosphorylation has been shown to cause the EphA2 cytoplasmic tail to interact with the PTB and SH2 domains of SHC, promote nuclear translocation and phosphorylation of ERK kinases, and increase nuclear induction of the Elk-1 transcription factor (Pratt and Kinch, 2002, Oncogene 21:7690-9). In another embodiment, EphA2/EphrinA1 Modulators decrease ligand-mediated EphA2 signaling. In a specific embodiment, EphA2/EphrinA1 Modulators decrease ligand-mediated EphA2 interaction with SHC. In another specific embodiment, EphA2/EphrinA1 Modulators decrease ligand-mediated nuclear translocation and/or phosphorylation of ERK kinases. In another specific embodiment, EphA2/EphrinA1 Modulators decrease ligand-mediated nuclear induction of the Elk-1 transcription factor. Any method in the art to assay ligand-mediated EphA2 signaling can be used to screen EphA2/EphrinA1 Modulators to determine their ability to decrease ligand-mediated EphA2 signaling, e.g., reporter gene assay, immunoprecipitation, immunoblotting, GST fusion protein pull down assay (see, e.g., Pratt and Kinch, 2002, Oncogene 21:7690-9).

4.6.2 EphA2/EphrinA1 Modulators that Increase EphrinA1 Enzymatic Activity

The invention provides methods of assaying and screening for EphA2/EphrinA1 Modulators that increase the enzymatic activity of EphrinA1. Such EphA2/EphrinA1 Modulators are identified by assaying for the ability of a candidate EphA2/EphrinA1 Modulator to increase the level of EphrinA1 enzymatic activity that is present in an EphrinA1-expressing cell, particularly an epithelial and/or endothelial cell. In some embodiments, the candidate agents are screened for ability to increase EphrinA1 enzymatic activity that is present when EphrinA1 is not bound to its receptor (e.g., EphA2). In other embodiments, candidate agents are screened for the ability to increase signaling through the EphrinA1 signaling cascade (e.g., in a reporter gene assay such as a CATalyse Reporter Gene Assay available from Serologicals Corporation, Norcross, Ga.) that is active when EphrinA1 is not bound to its receptor (e.g., EphA2).

4.6.3 EphA2/EphrinA1 Modulators that Decrease EphA2-Endogenous Ligand Interaction

The invention provides methods of assaying and screening for EphA2/EphrinA1 Modulators that decrease or disrupt EphA2-endogenous ligand interaction. In one embodiment, the EphA2/EphrinA1 Modulators (preferably one that possesses a structurally or functionally similar epitope as EphA2 or EphrinA1) are screened for ability to competitively bind cellular EphA2 or EphrinA1 so as to disrupt interaction/binding between cellular EphA2 and cellular EphrinA1 on cells that express EphA2 or EphrinA1. EphA2 or EphrinA1 binding to such a non-endogenous ligand preferably does not result in the type or degree of signaling that EphA2 binding its endogenous ligand elicits. In another embodiment, the EphA2/EphrinA1 Modulators (preferably a soluble endogenous ligand binding extracellular domain of EphA2 or EphrinA1) are screened for ability to competitively bind EphA2 or EphrinA1 so inhibit EphrinA1 interaction with cellular EphA2. The number of EphA2/EphrinA1 Modulators that competitively bind EphrinA1 or cellular EphA2 can be analyzed by various known techniques including, but not limited to, ELISAs, immunoblots, radio-immunoprecipitations, etc. The invention provides compositions wherein the percentage binding between cellular EphA2 and its endogenous ligand EphrinA1 is less than 99%, 95%, 90%, 80%, 70%, 60%, 50%, 40%, 30%, 20%, 10%, 5%, 1% relative to control in a protein-protein interaction assay as described in Section 4.2.2. In a preferred embodiment, the EphA2/EphrinA1 Modulators are screened for their ability to prevent or slow angiogenesis related to non-neoplastic hyperproliferative epithelial and/or endothelial cell disorders, including but not limited to disorders associated with increased deposition of extracellular matrix components (e.g., collagen, proteoglycans and fibronectin). Non-limiting examples of such disorders include cirrhosis, fibrosis (e.g., fibrosis of the liver, kidney, lungs, heart, retina and other viscera) and fibrosis-related diseases.

4.6.4 Cell Proliferation Stimulative Agents

The invention provides methods of assaying and screening for EphA2/EphrinA1 Modulators of the invention that promote proliferation/growth/survival of EphA2-expressing cells, particularly epithelial and/or endothelial cells. Many assays well-known in the art can be used to assess survival, growth, and/or proliferation; for example, cell proliferation can be assayed by measuring (3H)-thymidine incorporation, by direct cell count, by detecting changes in transcription, translation or activity of known genes such as cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as western blotting or immunoprecipitation using commercially available antibodies (for example, many cell cycle marker antibodies are from Santa Cruz Inc.). mRNA can be quantitated by methods that are well known and routine in the art, for example by northern analysis, RNase protection, the polymerase chain reaction in connection with the reverse transcription, etc. Cell viability can be assessed by using trypan-blue staining or other cell death or viability markers known in the art.

The present invention provides for cell cycle and cell proliferation analysis by a variety of techniques known in the art, including but not limited to the following:

As one example, bromodeoxyuridine (BRDU) incorporation may be used as an assay to identify proliferating cells. The BRDU assay identifies a cell population undergoing DNA synthesis by incorporation of BRDU into newly synthesized DNA. Newly synthesized DNA may then be detected using an anti-BRDU antibody (see Hoshino et al., 1986, Int. J Cancer 38:369; Campana et al., 1988, J. Immunol. Meth. 107:79).

Cell proliferation may also be examined using (3H)-thymidine incorporation (see e.g., Chen, 1996, Oncogene 13:1395-403; Jeoung, 1995, J. Biol. Chem. 270:18367-73). This assay allows for quantitative characterization of S-phase DNA synthesis. In this assay, cells synthesizing DNA will incorporate (³H)-thymidine into newly synthesized DNA. Incorporation may then be measured by standard techniques in the art such as by counting of radioisotope in a Scintillation counter (e.g. Beckman LS 3800 Liquid Scintillation Counter).

Detection of proliferating cell nuclear antigen (PCNA) may also be used to measure cell proliferation. PCNA is a 36 kDa protein whose expression is elevated in proliferating cells, particularly in early G1 and S phases of the cell cycle and therefore may serve as a marker for proliferating cells. Positive cells are identified by immunostaining using an anti-PCNA antibody (see Li et al., 1996, Curr. Biol. 6:189-99; Vassilev et al., 1995, J. Cell Sci. 108:1205-15).

Cell proliferation may be measured by counting samples of a cell population over time (e.g. daily cell counts). Cells may be counted using a hemacytometer and light microscopy (e.g. HyLite hemacytometer, Hausser Scientific). Cell number may be plotted against time in order to obtain a growth curve for the population of interest. In a preferred embodiment, cells counted by this method are first mixed with the dye Trypan-blue (Sigma), such that living cells exclude the dye, and are counted as viable members of the population.

DNA content and/or mitotic index of the cells may be measured, for example, based on the DNA ploidy value of the cell. For example, cells in the G1 phase of the cell cycle generally contain a 2N DNA ploidy value. Cells in which DNA has been replicated but have not progressed through mitosis (e.g. cells in S-phase) will exhibit a ploidy value higher than 2N and up to 4N DNA content. Ploidy value and cell-cycle kinetics may be further measured using propidum iodide assay (see e.g. Turner, et al., 1998, Prostate 34:175-81). Alternatively, the DNA ploidy may be determined by quantitation of DNA Feulgen staining (which binds to DNA in a stoichiometric manner) on a computerized microdensitometrystaining system (see e.g., Bacus, 1989, Am. J. Pathol. 135:783-92). In an another embodiment, DNA content may be analyzed by preparation of a chromosomal spread (Zabalou, 1994, Hereditas. 120:127-40; Pardue, 1994, Meth. Cell Biol. 44:333-351).

The expression of cell-cycle proteins (e.g., CycA. CycB, CycE, CycD, cdc2, Cdk4/6, Rb, p21, p27, etc.) provide crucial information relating to the proliferative state of a cell or population of cells. For example, identification in an anti-proliferation signaling pathway may be indicated by the induction of p21^{cip1}. Increased levels of p21 expression in cells results in delayed entry into G1 of the cell cycle (Harper et al., 1993, Cell 75:805-816; Li et al., 1996, Curr. Biol. 6:189-199). p21 induction may be identified by immunostaining using a specific anti-p21 antibody available commercially (e.g. Santa Cruz). Similarly, cell-cycle proteins may be examined by western blot analysis using commercially available antibodies. In another embodiment, cell populations are synchronized prior to detection of a cell cycle protein. Cell cycle proteins may also be detected by FACS (fluorescence-activated cell sorter) analysis using antibodies against the protein of interest.

EphA2/EphrinA1 Modulators of the invention can also be identified by their ability to change the length of the cell cycle or speed of cell cycle so that cell proliferation is decreased or inhibited. In one embodiment the length of the cell cycle is determined by the doubling time of a population of cells (e.g., using cells contacted or not contacted with one or more candidate EphA2 agents). In another embodiment, FACS analysis is used to analyze the phase of cell cycle progression, or purify G1, S, and G2/M fractions (see e.g., Delia et al., 1997, Oncogene 14:2137-47).

4.6.5 EphA2/EphrinA1 Modulators that Increase Integrity of Cell Layer

The invention provides methods of assaying and screening for EphA2/EphrinA1 Modulators of the invention that increase the maintenance or reconstitution of the integrity of a cell layer, especially an epithelial and/or endothelial cell layer. Candidate agents are screened for their ability to maintain and/or reconstitute epithelial and/or endothelial cell layer integrity in a bicameral chamber (e.g., Boyden chamber, Ussing chamber, Tranwell chamber, etc.). For example, a bicameral chamber can be set up such that a monolayer of epithelial cells is present between an upper and lower well of medium. Cell layer integrity in the presence and absence of candidate EphA2 agents can be ascertained by a number of methods. For example, the degree of passive solute flow between chamber wells can be indicative of cell layer integrity. A marker molecule (e.g., stain, radioactive label) can be added to one of the wells and the time period it takes for the marker molecule to have access to the medium in the other well can be measured. Alternatively, the transepithelial electrical resistance may be measured to indicate the cell layer integrity. Increasing cell layer integrity is indicated by increasing transepithelial electrical resistance. See generally, Kim & Suh, 1993, Am. J. Physiol. 264:L308-15 and Nilsson et al., 1996, Eur. J. Endocrinol. 135:469-80.

4.6.6 Agents that Inhibit Pathology-Causing Epithelial or Endothelial Cell Phenotypes

Phenotypes

EphA2/EphrinA1 Modulators of the invention may reduce (and preferably inhibit) one or pathology-causing epithelial or endothelial cell phenotypes (e.g., mucin secretion, differentiation into mucin-secreting cells, secretion of inflammatory factors, secretion of ECM factors, particularly fibronectin, hyperproliferation, and/or aberrant angiogenesis). One of skill in the art can assay candidate EphA2/EphrinA1 Modulators for their ability to reduce (and preferably inhibit) such behavior. In specific embodiments, an EphA2/EphrinA1 Modulator reduces (and preferably inhibits) a pathology-causing epithelial or endothelial cell phenotype by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control (e.g., PBS or IgG).

In some embodiments, in vitro models of lung epithelia can be used to screen candidate agents. Cells can be cultured to form a pseudo-stratified, highly differentiated model tissue from human-derived tracheal/bronchial epithelial cells (e.g., NHBE or TBE cells) which closely resembles the epithelial tissue of the respiratory tract. The cultures can be grown on cell culture inserts at the air-liquid interface, allowing for gas phase exposure of volatile materials in airway inflammation and irritancy studies, as well as in inhalation toxicity studies. Transepithelial permeability can be measured for inhaled drug delivery studies. Such model systems are available commercially such as EpiAirway™ Tissue Model System (MatTek Corp., Ashland, Mass.).

Mucin Secretion

In one embodiment, the pathology-causing epithelial cell phenotype is mucin secretion. Candidate EphA2/EphrinA1 Modulators can be assayed for their ability to decrease or inhibit mucin secretion by a number of in vitro and in vivo assays. One example of an in vitro assay that can be used to measure mucin release from cultured airway goblet cells is a hamster tracheal surface epithelial (HTSE) cell culture system (see U.S. Pat. No. 6,245,320). Briefly, tracheas obtained from 7-8 week old male Golden Syrian hamsters (Harlan Sprague Dawley, Indianapolis, Ind.) are used to harvest HTSE cells. HTSE cells are then cultured on a collagen gel as described in Kim et al., 1989, Exp. Lung Res. 15:299-314. Mucins are metabolically radiolabeled by incubating confluent cultures with labeling medium for 24 hours as described in Kim et al., 1989, Am. J Resp. Cell Mol. Biol. 1:137-143. At the end of the 24 hour incubation period, the spent media (the pretreatment sample) is collected, and the labeled cultures are washed twice with PBS without Ca⁺⁺ and Mg⁺⁺ and then chased for 30 min in the presence of candidate EphA2/EphrinA1 Modulators. The chased media are referred to as the treatment samples. At the end of the chase period, floating cells and cell debris are removed from the treatment samples by centrifugation and assayed for their labeled mucin content. High molecular weight glycoconjugates that are excluded after Sepharose CL-4B (Pharmacia, Upsala, Sweden) gel-filtration column chromatography and that are resistant to hyaluronidase are defined as mucins (see Kim et al., 1985, J. Biol. Chem. 260:4021:4027). Mucins are then measured by column chromatography as described in Kim et al., 1987, PNAS 84:9304-9308. The amount of secreted mucin in HTSE cultures before and after incubation with a candidate EphA2/EphrinA1 Modulator can be determined.

Other in vitro assays can be used, such as primary tracheal epithelial cell cultures maintained in an air/liquid interface system that maintains differentiated characteristics (Adler et al., 1992, Am. J. Respir. Cell Mol. Biol. 6:550-556) and lung epithelial cell lines (e.g., NIH-292 cells). Standard molecular biological techniques can be use to determine mucin amount, including but not limited to, western blot and ELISA for protein expression levels and PCR and northern blots for RNA expression levels.

In vivo assays can also be used to identify EphA2/EphrinA1 Modulators of the invention. Animal models for asthma or COPD can also be used to identify EphA2/EphrinA1 Modulators of the invention. For example, a murine model of endotoxin/LPS-induced lung inflammation can be used to assay the affect of candidate EphA2 agonistic agents on differentiation of mucin-secreting cells (Steiger et al., 1995, J. Am. Respir. Cell Mol. Biol., 12:307-14 and U.S. Pat. No. 6,083,973). Briefly, lung inflammation can be induced in mice or rats by repeated instillation of LPS (LPS derived from Pseudomonas aeriginos; Sigma Chemical) 400 μg/kg/dose/day for three days. Animals can be treated with a candidate EphA2/EphrinA1 Modulator once daily, starting 24 hours prior to the first LPS challenge. Animals are sacrificed 24 hours after the last LPS challenge by exsanguination under deep anesthesia. The lungs are lavaged with phosphate buffered saline (2×5 ml) to wash out mucous layer. The bronchial lavage fluid is centrifuged for 10 min and the cell-free supernate is frozen and stored −20° C. until analysis to determine the amount of mucin present. Amount of mucin secretion can be determined by any method known in the art, e.g., by dot blot assay using Alcian-blue and/or periodic acid-Schiff stains or by western blot/ELISA analysis using anti-mucin antibodies.

Other animal models of asthma/COPD can also be used to identify EphA2/EphrinA1 Modulators such as mice that overexpress IL-4 (Temann et al., 1997, Am. J. Respir. Cell Mol. Biol. 16:471-8), IL-13 (Kuperman, et al., 2002, Nat. Med. July 1, epub ahead of print) or IL-9 either systemically or only in lung tissue. Reduction in pathological symptoms can be used to identify EphA2/EphrinA1 Modulators as well as a decreased amount of mucin present in bronchial lavage fluid or induced sputum samples (Fahy et al., 1993, Am. Rev. Respir. Dis. 147:1132-1137). Another example of an animal model is the murine adoptive transfer model in which aeroallergen provocation of TH1 or TH2 recipient mice results in TH effector cell migration to the airways and is associated with an intense neutrophilic (TH1) and eosinophilic (TH2) lung mucosal inflammatory response (Cohn et al., 1997, J. Exp. Med. 1861737-1747). For a review of animal models of COPD see Szelenyi and Marx, 2001, Arzneimittelforschung 51:1004-14.

Differentiation into Mucin-Secreting Cells

In one embodiment, the pathology-causing epithelial cell phenotype is differentiation into mucin-secreting cells (e.g., goblet cells). Candidate EphA2/EphrinA1 Modulators can be assayed (both in vitro and in vivo) for their ability to decrease or inhibit epithelial cell differentiation to mucin-secreting cells. Animal models for asthma or COPD can be used to identify EphA2/EphrinA1 Modulators of the invention. For example, animals with LPS-induced lung inflammation can be used to assay the affect of candidate EphA2/EphrinA1 Modulators on differentiation of mucin-secreting cells (see U.S. Pat. No. 6,083,973). Animals with LPS-induced lung inflammation that were either treated with a candidate EphA2/EphrinA1 Modulator or were an untreated control are sacrificed before lung perfusion with 10% neutral buffered formalin by intratracheal instillation at a constant rate (5 ml at 1 ml/min). The lung lobes are then excised and immersed in fixative for 24 hours prior to processing. Standard methods can be used to prepare 5 μm paraffin sections. Sections are stained with Alcian blue (pH 2.5) and/or periodic acid/Schiffs reagent and/or anti-mucin antibodies to detect mucosubstances within the lung tissue. Morphometric analysis for goblet hyperplasia can performed by counting all airways ≧2 mm in diameter and determining the percentage of airways that contain positively stained cells.

Secretion of Inflammatory Factors

In one embodiment, the pathology-causing epithelial or endothelial cell phenotype is secretion of inflammatory factors. Although mast cells and eosinophils may initially release mediators of the inflammatory response, epithelial cells in hyperproliferative disorders do alter their phenotype to one that secretes cytokines and chemokines (Holgate et al., 1999, Clin. Exp. Allergy 29:90-5). Any method known in the art to assay for cytokine/chemokine production or secretion can be used to quantitate differences in in vitro or in vivo epithelial or endothelial cells that have been either treated or untreated with candidate EphA2/EphrinA1 Modulators. In certain embodiments, IL-4, IL-9, and/or IL-13 production or secretion are assessed.

Non-Neoplastic Hyperproliferation

In one embodiment, the pathology-causing epithelial or endothelial cell phenotype is non-neoplastic hyperproliferation. Many assays well-known in the art can be used to assess survival, growth and/or proliferation; for example, cell proliferation can be assayed by measuring (3H)-thymidine incorporation, by direct cell count, by detecting changes in transcription, translation or activity of known genes such as cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as western blotting or immunoprecipitation using commercially available antibodies (for example, many cell cycle marker antibodies are from Santa Cruz Inc.). mRNA can be quantitated by methods that are well known and routine in the art, for example by northern analysis, RNase protection, the polymerase chain reaction in connection with the reverse transcription, etc. Cell viability can be assessed by using trypan-blue staining or other cell death or viability markers known in the art.

The present invention provides for cell cycle and cell proliferation analysis by a variety of techniques known in the art, including but not limited to the following:

As one example, bromodeoxyuridine (BRDU) incorporation may be used as an assay to identify proliferating cells. The BRDU assay identifies a cell population undergoing DNA synthesis by incorporation of BRDU into newly synthesized DNA. Newly synthesized DNA may then be detected using an anti-BRDU antibody (see Hoshino et al., 1986, Int. J. Cancer 38:369; Campana et al., 1988, J. Immunol. Meth. 107:79).

Cell proliferation may also be examined using (3H)-thymidine incorporation (see e.g., Chen, 1996, Oncogene 13:1395-403; Jeoung, 1995, J. Biol. Chem. 270:18367-73). This assay allows for quantitative characterization of S-phase DNA synthesis. In this assay, cells synthesizing DNA will incorporate (3H)-thymidine into newly synthesized DNA. Incorporation may then be measured by standard techniques in the art such as by counting of radioisotope in a Scintillation counter (e.g. Beckman LS 3800 Liquid Scintillation Counter).

Detection of proliferating cell nuclear antigen (PCNA) may also be used to measure cell proliferation. PCNA is a 36 kilodalton protein whose expression is elevated in proliferating cells, particularly in early G1 and S phases of the cell cycle and therefore may serve as a marker for proliferating cells. Positive cells are identified by immunostaining using an anti-PCNA antibody (see Li et al., 1996, Curr. Biol. 6:189-99; Vassilev et al., 1995, J. Cell Sci. 108:1205-15).

Cell proliferation may be measured by counting samples of a cell population over time (e.g. daily cell counts). Cells may be counted using a hemacytometer and light microscopy (e.g. HyLite hemacytometer, Hausser Scientific). Cell number may be plotted against time in order to obtain a growth curve for the population of interest. In a preferred embodiment, cells counted by this method are first mixed with the dye Trypan-blue (Sigma), such that living cells exclude the dye, and are counted as viable members of the population.

DNA content and/or mitotic index of the cells may be measured, for example, based on the DNA ploidy value of the cell. For example, cells in the G1 phase of the cell cycle generally contain a 2N DNA ploidy value. Cells in which DNA has been replicated but have not progressed through mitosis (e.g. cells in S-phase) will exhibit a ploidy value higher than 2N and up to 4N DNA content. Ploidy value and cell-cycle kinetics may be further measured using propidum iodide assay (see e.g. Turner, et al., 1998, Prostate 34:175-81). Alternatively, the DNA ploidy may be determined by quantitation of DNA Feulgen staining (which binds to DNA in a stoichiometric manner) on a computerized microdensitometrystaining system (see e.g., Bacus, 1989, Am. J. Pathol. 135:783-92). In an another embodiment, DNA content may be analyzed by preparation of a chromosomal spread (Zabalou, 1994, Hereditas. 120:127-40; Pardue, 1994, Meth. Cell Biol. 44:333-351).

The expression of cell-cycle proteins (e.g., CycA. CycB, CycE, CycD, cdc2, Cdk4/6, Rb, p21, p27, etc.) provide crucial information relating to the proliferative state of a cell or population of cells. For example, identification in an anti-proliferation signaling pathway may be indicated by the induction of p21^{cip1}. Increased levels of p21 expression in cells results in delayed entry into G1 of the cell cycle (Harper et al., 1993, Cell 75:805-816; Li et al., 1996, Curr. Biol. 6:189-199). p21 induction may be identified by immunostaining using a specific anti-p21 antibody available commercially (e.g. Santa Cruz). Similarly, cell-cycle proteins may be examined by western blot analysis using commercially available antibodies. In another embodiment, cell populations are synchronized prior to detection of a cell cycle protein. Cell cycle proteins may also be detected by FACS (fluorescence-activated cell sorter) analysis using antibodies against the protein of interest.

EphA2/EphrinA1 Modulators of the invention can also be identified by their ability to change the length of the cell cycle or speed of cell cycle so that cell proliferation is decreased or inhibited. In one embodiment the length of the cell cycle is determined by the doubling time of a population of cells (e.g., using cells contacted or not contacted with one or more candidate EphA2/EphrinA1 Modulators). In another embodiment, FACS analysis is used to analyze the phase of cell cycle progression, or purify G1, S, and G2/M fractions (see e.g., Delia et al., 1997, Oncogene 14:2137-47).

4.6.7 EphA2/EphrinA1 Modulators that Inhibit Pathology-Causing Endothelial Cell Phenotypes

EphA2/EphrinA1 Modulators of the invention may preferably reduce (and preferably inhibit) pathology-causing endothelial cell phenotypes (e.g., increased cell migration (not including metastasis), increased cell volume, secretion of extracellular matrix molecules (e.g., collagen, fibronectin, tenascin, proteoglycans, etc.) or matrix metalloproteinases (e.g., gelatinases, collagenases, and stromelysins), hyperproliferation, and increased angiogenesis). One of skill in the art can assay candidate EphA2/EphrinA1 Modulators for their ability to reduce (and preferably inhibit) such behavior. In specific embodiments, an EphA2/EphrinA1 Modulator reduces (and preferably inhibits) a pathology-causing endothelial cell phenotype by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control (e.g., PBS or IgG).

Cell Migration

In one embodiment, the pathology-causing endothelial cell phenotype is increased cell migration (not including metastasis). Candidate EphA2/EphrinA1 Modulators can be assayed (both in vitro and in vivo) for their ability to decrease or inhibit endothelial cell migration. Any assay known in the art can be used to measure endothelial cell migration. For example, migration can be evaluated in a Boyden chamber migration assay. Briefly, endothelial cells (e.g., smooth muscle cell) can be added to the upper well of the chamber. Following cell attachment, one or more candidate EphA2/EphrinA1 Modulators can be added to the upper chamber. Cells can be allowed to migrate to the lower chamber either with or without an attracted (e.g., PDGF) added to the medium of the lower chamber. Cells which migrated through to the lower chamber can be stained and counted.

Secretion of Extracellular Matrix Molecules such as Fibronectin and Matrix Metalloproteinases

In one embodiment, the pathology-causing endothelial cell phenotype is secretion of extracellular matrix molecules, such as fibronectin, or matrix metalloproteinases. Any method known in the art to assay for extracellular matrix molecule and matrix metalloproteinase production or secretion can be used to quantitate differences in in vitro or in vivo endothelial cells that have been either treated or untreated with candidate EphA2/EphrinA1 Modulators. For example, western or northern blot analysis, reverse transcription-polymerase chain reaction, or ELISA assays can be used to quantitate expression levels. The activity of matrix metalloproteinases can be assayed by any method known in the art including zymography (see, e.g., Badier-Commander, 2000, J. Pathol. 192:105-112).

In one specific embodiment, the ability to decrease expression level and/or activity level of gelatinase-A (also known as MMP-2) is used to screen for EphA2/EphrinA1 Modulators of the invention. In another embodiment, the ability to modulate fibronectin expression is used to screen for EphA2/EphrinA1 Modulators of the invention.

Non-Neoplastic Hyperproliferation

In one embodiment, the pathology-causing endothelial cell phenotype is non-neoplastic hyperproliferation and/or aberrant angiogenesis. Many assays well-known in the art can be used to assess survival, growth and/or proliferation. Any in vitro assay listed in Section 4.6 can be used to assess growth, proliferation and/or cell survival of endothelial cells in the presence and absence of candidate EphA2/EphrinA1 Modulators. Animal models of endothelial cell hyperproliferation can also be used. For example, New Zealand White rabbits can be used for an in vivo model of restenosis (see e.g., Feldman et al, 2000, Circulation; 101:908-16; Feldman et al., 2001, Circulation 103:3117-22; Frederick et al., 2001, Circulation 104:3121-4). Briefly, bilateral iliac artery balloon angioplasty is performed with a 3-mm-diameter balloon (3×1-minute inflation, 10 atm); then a 15-mm-long Crown stent (Cordis) mounted over the balloon was implanted in the right iliac artery only (30-second inflation, 10 atm). Animals are euthanized at 1, 3, 7, 30, or 60 days after injury. At each time point, right (stent) and left (balloon angioplasty) iliac arteries were harvested, flushed with ice-cold saline, cleaned of any adipose tissue, and divided into 2 or 3 segments. Morphometric analyses and immunohistochemistry are performed on the excised arteries. Stented and nonstented arterial segments are fixed in 4% paraformaldehyde. Morphometric analyses are performed on hematoxylin-phloxin-safran-stained cross sections of the arteries. For immunohistochemistry, arterial segments are embedded in OCT compound, frozen in liquid nitrogen and chilled isopentane after stent struts are removed with microforceps. Four-micrometer cross sections are obtained from each block and immunostained, e.g., with anti extracellular matrix molecule or anti-matrix metalloproteinase antibodies.

Angiogenesis

Candidate EphA2/EphrinA1 Modulators can be assayed (both in vitro and in vivo) for their ability to modulate angiogenesis. Many assays are well known in the art to assess angiogenesis or angiogenic activity. For a general review of angiogenesis assays, see, e.g., Auerbach et al., 2003, Clinical Chemistry 49:32-40, which is incorporated by reference herein in its entirety. For example, mouse corneal angiogensis assays may be performed (see, e.g., Cheng et al., Mol. Cancer Res., 2002, 1:2-11 and Kenyon et al., 1996, Invest. Ophthalmol. Vis. Sci. 37:1625-1632). Briefly, hydron pellents containing sucralfate with either vehicle alone (PBS or IgG), an angiogenic factor (e.g., bFGF, VEGF) or an EphA2/EphrinA1 Modulator of the invention is prepared. Pellets are surgically implanted into corneal micropockets created 1 mm to the lateral corneal limbus of a mouse (e.g., C57/BL6; The Jackson Laboratory, Bar Harbor, Me.). At day 5 post-implantation, comeas are photographed at an incipient angle of 35-50° from the polar axis in the meridian containing the pellet, using a Zeiss split lamp. The fraction of the total corneal image that is vascularized (VA), and the ratio of pixels marking neovascular capillaries both within the vascularized region (RVD) and within the total corneal image (TVD) is calculated using Bioquant software (Vanderbilt University, Nashville, Tenn.). Statistical analysis may be performed by using the two-tailed, paired Student's t test. Other non-limiting examples of angiogenesis assays that can be used to identify candidate EphA2/EphrinA1 Modulator agents that modulate angiogenesis include CAM assays, matrigel plug assays, endothelial cell migration assays, tube formation assays, aortic ring assays, and chick aortic arch assays (Auerbach et al., 2003, Clinical Chemistry 49:32-40).

4.7 Biological Activity of Therapies

4.7.1 Toxicity

Toxicity and efficacy of the prophylactic and/or therapeutic protocols of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

4.7.2 Assays

The protocols and compositions of the invention are preferably tested in vitro, and then in vivo, for the desired therapeutic or prophylactic activity, prior to use in humans. For example, in vitro assays which can be used to determine whether administration of a specific prophylactic or therapeutic protocol is indicated, include in vitro cell culture assays in which a patient tissue sample is grown in culture, and exposed to or otherwise administered a protocol, and the effect of such protocol upon the tissue sample is observed, e.g., decreased EphA2-endogenous ligand binding, decreased EphrinA1 gene expression, upregulated EphA2 gene expression, increased EphA2 protein stability or protein accumulation, decreased EphA2 cytoplasmic tail phosphorylation, increased proliferation of EphA2 expressing cells, increased survival of EphA2 expressing cells, maintained/reconstituted integrity of an epithelial and/or endothelial cell layer, decreased deposition of ECM components (e.g., collagen), and/or decreased angiogenesis. A demonstration of any of the aforementioned properties of the contacted cells indicates that the therapeutic agent is effective to treat the condition in the patient. Alternatively, instead of culturing cells from a patient, therapeutic agents and methods may be screened using cells of a epithelial and/or endothelial cell line. Many assays standard in the art can be used to assess such survival, growth, and/or proliferation; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining.

In some embodiments, where the disorder is a non-neoplastic hyperproliferative lung epithelial cell disorder, in vitro models of lung epithelia can be used to demonstrate prophylactic/therapeutic utility of the protocols and compositions of the invention. Cells can be cultured to form a pseudo-stratified, highly differentiated model tissue from human-derived tracheal/bronchial epithelial cells (e.g., NHBE or TBE cells) which closely resembles the epithelial tissue of the respiratory tract. The cultures can be grown on cell culture inserts at the air-liquid interface, allowing for gas phase exposure of volatile materials in airway inflammation and irritancy studies, as well as in inhalation toxicity studies. Transepithelial permeability can be measured for inhaled drug delivery studies. Such model systems are available commercially such as EpiAirway™ Tissue Model System (MatTek Corp., Ashland, Mass.). In some embodiments, the cell cultures are exposed to or otherwise administered a therapeutic and/or prophylactic protocol of the invention and the effect of such protocol upon the cell culture is observed, e.g., decreased EphA2-endogenous ligand binding, decreased EphrinA1 gene expression and/or translation, upregulated EphA2 gene expression and/or translation, increases EphA2 protein stability or protein accumulation, decreased EphA2 cytoplasmic tail phosphorylation, increased proliferation of EphA2 expressing cells, increased survival of EphA2 expressing cells, maintained/reconstituted integrity of an epithelial and/or endothelial cell layer, decreased deposition of ECM components (e.g., collagen), and/or decreased angiogenesis. A demonstration of any of the aforementioned properties of the contacted cells indicates that the therapeutic agent is effective to treat the non-neoplastic hyperproliferative lung epithelial cell disorder. In addition, assays standard in the art can be used to assess cell survival, growth, and/or proliferation; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining.

In other embodiments, the disorder is lung fibrosis and the in vitro model is Beas-2B cells (bronchial epithelium cells transformed with SV40 virus) treated with bleomycin. In another embodiment, an in vivo model for lung fibrosis is bleomycin treatment of susceptible strains of mice. Bleomycin induces lung epithelial cell death, followed by acute neutrophilic influx, subsequent chronic inflammation, and parenchymal fibrosis in mice. Bleomycin-treated lung epithelial cells as a model for lung fibrosis replicates key pathologic features of human lung fibrotic diseases such as IPF. In some embodiments, the bleomycin-treated Beas-2B cells or bleomycin-treated mice are exposed to or otherwise administered a therapeutic or prophylactic protocol of the invention, and the effect of such protocol upon the cell culture or tissue sample from such bleomycin-treated mice is observed, e.g., decreased EphA2-endogenous ligand binding, decreased EphrinA1 gene expression and/or translation, upregulated EphA2 gene expression and/or translation, increases EphA2 protein stability or protein accumulation, decreased EphA2 cytoplasmic tail phosphorylation, increased proliferation of EphA2 expressing cells, increased survival of EphA2 expressing cells, maintained/reconstituted integrity of an epithelial and/or endothelial cell layer, decreased deposition of ECM components (e.g., collagen), and/or decreased angiogenesis. A demonstration of any of the aforementioned properties of the contacted cells indicates that the therapeutic agent is effective to treat the non-neoplastic hyperproliferative lung epithelial cell disorder. In addition, assays standard in the art can be used to assess cell survival, growth, and/or proliferation; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining.

Compounds for use in therapy can be tested in suitable animal model systems prior to testing in humans, including but not limited to in rats, mice, chicken, cows, monkeys, rabbits, hamsters, etc. The compounds can then be used in the appropriate clinical trials. In a preferred embodiment of the invention, an animal model for lung fibrosis is bleomycin treatment of susceptible strains of mice.

Further, any assays known to those skilled in the art can be used to evaluate the prophylactic and/or therapeutic utility of the combinatorial therapies disclosed herein for treatment or prevention of a non-neoplastic hyperproliferative epithelial and/or endothelial cell disorder, such as fibrosis (e.g., fibrosis of the liver, kidney, lungs, heart, retina and other viscera) or a fibrosis-related disease.

4.7.3 Dosages

The amount of the composition of the invention which will be effective in the treatment, management, or prevention of non-neoplastic hyperproliferative epithelial and/or endothelial cell disorders, including but not limited to cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis, can be determined by standard research techniques. For example, the dosage of the composition which will be effective in the treatment, management, or prevention of any of the above diseases, can be determined by administering the composition to an animal model such as, e.g., the animal models known to those skilled in the art (e.g., bleomycin-treated mouse models). In addition, in vitro assays may optionally be employed to help identify optimal dosage ranges.

Selection of the preferred effective dose can be determined (e.g., via clinical trials) by a skilled artisan based upon the consideration of several factors which will be known to one of ordinary skill in the art. Such factors include the disorder to be treated or prevented, the symptoms involved, the patient's body mass, the patient's immune status and other factors known by the skilled artisan to reflect the accuracy of administered pharmaceutical compositions.

The precise dose to be employed in the formulation will also depend on the route of administration, and the seriousness of the non-neoplastic hyperproliferative epithelial and/or endothelial cell disorder, including but not limited to cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis, and should be decided according to the judgment of the practitioner and each patient's circumstances. Effective doses may be extrapolated from dose-response curves derived from in vitro or animal model test systems.

For antibodies, proteins, polypeptides, peptides and fusion proteins encompassed by the invention, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg, 0.01 to 0.10 mg/kg, 0.1 mg/kg or 20 mg/kg of the patient's body weight, more preferably 1 mg/kg to 10 mg/kg of the patient's body weight. Generally, human and humanized antibodies have a longer half-life within the human body than antibodies from other species due to the immune response to the foreign polypeptides. Thus, lower dosages of human antibodies and less frequent administration is often possible. Further, the dosage and frequency of administration of antibodies, proteins, polypeptides, peptides and fusion proteins encompassed by the invention or fragments thereof may be reduced by enhancing uptake and tissue penetration of the antibodies by modifications such as, for example, lipidation.

For small molecules, exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

For other therapies administered to a patient, the typical doses of various immunomodulatory therapeutics are known in the art. Given the invention, certain preferred embodiments will encompass the administration of lower dosages in combination treatment regimens than dosages recommended for the administration of single agents.

In certain embodiments, the EphA2- or EphrinA1 antigenic peptides and anti-idiotypic antibodies of the invention are formulated at 1 mg/ml, 5 mg/ml, 10 mg/ml, and 25 mg/ml for intravenous injections and at 5 mg/ml, 10 mg/ml, and 80 mg/ml for repeated subcutaneous administration and intramuscular injection.

Where the EphA2/EphrinA1 Modulator is a bacterial vaccine, the vaccine can be formulated at amounts ranging between approximately 1×10² CFU/ml to approximately 1×10¹² CFU/ml, for example at 1×10² CFU/ml, 5×10² CFU/ml, 1×10³ CFU/ml, 5×10³ CFU/ml, 1×10⁴ CFU/ml, 5×10⁴ CFU/ml, 1×10⁵ CFU/ml, 5×10⁵ CFU/ml, 1×10⁶ CFU/ml, 5×10⁶ CFU/ml, 1×10⁷ CFU/ml, 5×10⁷ CFU/ml, 1×10⁸ CFU/ml, 5×10⁸ CFU/ml, 1×10⁹ CFU/ml, 5×10⁹ CFU/ml, 1×10¹⁰ CFU/ml, 5×10¹⁰ CFU/ml, 1×10¹¹ CFU/ml, 5×10¹¹ CFU/ml, or 1×10¹² CFU/ml.

For EphA2- and EphrinA1 antigenic peptides or anti-idiotypic antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight.

With respect to the dosage of bacterial EphA2- and EphrinA1 vaccines of the invention, the dosage is based on the amount colony forming units (c.f.u.). Generally, in various embodiments, the dosage ranges are from about 1.0 c.f.u./kg to about 1×10¹⁰ c.f.u./kg; from about 1.0 c.f.u./kg to about 1×10⁸ c.f.u./kg; from about 1×10² c.f.u./kg to about 1×10⁸ c.f.u./kg; and from about 1×10⁴ c.f.u./kg to about 1×10⁸ c.f.u./kg. Effective doses may be extrapolated from dose-response curves derived animal model test systems. In certain exemplary embodiments, the dosage ranges are 0.001-fold to 10,000-fold of the murine LD₅₀, 0.01-fold to 1,000-fold of the murine LD₅₀, 0.1-fold to 500-fold of the murine LD₅₀, 0.5-fold to 250-fold of the murine LD₅₀, 1-fold to 100-fold of the murine LD₅₀, and 5-fold to 50-fold of the murine LD₅₀. In certain specific embodiments, the dosage ranges are 0.00.1-fold, 0.01-fold, 0.1-fold, 0.5-fold, 1-fold, 5-fold, 10-fold, 50-fold, 100-fold, 200-fold, 500-fold, 1,000-fold, 5,000-fold or 10,000-fold of the murine LD₅₀.

The invention provides for any method of administrating lower doses of known therapies than previously thought to be effective for the prevention, treatment, management, or prevention of non-neoplastic hyperproliferative epithelial and/or endothelial cell disorders, including but not limited to cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis. Preferably, lower doses of known immunomodulatory are administered in combination with lower doses of EphA2/EphrinA1 Modulators of the invention.

4.8 Pharmaceutical Compositions

The compositions of the invention include bulk drug which is useful in the manufacture of oral pharmaceutical compositions (e.g., non-sterile compositions) and parenteral pharmaceutical compositions (i.e., compositions that are suitable for administration to a subject or patient which are sterile) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. Preferably, compositions of the invention comprise a prophylactically or therapeutically effective amount of one or more EphA2/EphrinA1 Modulators of the invention and a pharmaceutically acceptable carrier. In a further embodiment, the composition of the invention further comprises one or more prophylactic or therapeutic agents other than an EphA2/EphrinA1 Modulator of the invention, e.g., an immunomodulatory agent, an anti-inflammatory agent, an anti-angiogenic agent or a TNF-α antagonist.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient adjuvant (e.g., Freund's adjuvant or, more preferably, MF59C.1 adjuvant available from Chiron (Emeryville, Calif.), excipient, or vehicle with which the therapeutic is administered. Other such adjuvants may include, but are not limited to mineral gels such as aluminum hydroxide; surface active substances such as lysolecithin, pluronic polyols, polyanions; other peptides; oil emulsions; and potentially useful human adjuvants such as BCG and Corynebacterium parvum. The pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Various delivery systems are known and can be used to administer an EphA2/EphrinA1 Modulator of the invention or the combination of an EphA2/EphrinA1 Modulator of the invention and a non-EphA2/EphrinA1 Modulator therapy useful for preventing/treating non-neoplastic hyperproliferative epithelial and/or endothelial cell disorders, e.g., encapsulation in liposomes, microparticles, microcapsules, recombinant cells capable of expressing the antibody or antibody fragment, receptor-mediated endocytosis (see, e.g., Wu and Wu, 1987, J. Biol. Chem. 262:4429-4432), construction of a nucleic acid as part of a retroviral or other vector, etc.

Methods of administering a therapy (e.g., prophylactic or therapeutic agent) of the invention include, but are not limited to, parenteral administration (e.g., intradermal, intramuscular, intraperitoneal, intravenous and subcutaneous), epidural, and mucosal (e.g., intranasal, inhaled, and oral routes). In a specific embodiment, prophylactic or therapeutic agents of the invention are administered intramuscularly, intravenously, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic agents of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers.

In yet another embodiment, the prophylactic or therapeutic agent can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the antibodies of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; International Patent Publication Nos. WO 99/15154 and WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938; International Patent Publication Nos. WO 91/05548 and WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control. Rel. Bioact. Mater. 24:853-854; and Lam et al., 1997, Proc. Int'l. Symp. Control Rel. Bioact. Mater. 24:759-760, each of which is incorporated herein by reference in its entirety.

Pharmaceutical compositions for use in accordance with the present invention may be formulated in conventional manner using one or more physiologically acceptable carriers or excipients. Preferably, agents are formulated and administered systemically. Techniques for formulation and administration may be found in “Remington: The Science and Practice of Pharmacy”, 19th ed., 1995, Lippincott Williams & Wilkins, Baltimore, Md.

Thus, the EphA2/EphrinA1 Modulators of the invention and their physiologically acceptable salts and solvates may be formulated for administration by inhalation or insufflation (either through the mouth or the nose) or oral, parenteral or mucosal (such as buccal, vaginal, rectal, sublingual) administration. In a preferred embodiment, local or systemic parenteral administration is used.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the prophylactic or therapeutic agents for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The prophylactic or therapeutic agents may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The prophylactic or therapeutic agents may also be formulated in rectal compositions such as suppositories or retention enemas, e.g., containing conventional suppository bases such as cocoa butter or other glycerides.

In addition to the formulations described previously, the prophylactic or therapeutic agents may also be formulated as a depot preparation. Such long acting formulations may be administered by implantation (for example subcutaneously or intramuscularly) or by intramuscular injection. Thus, for example, the prophylactic or therapeutic agents may be formulated with suitable polymeric or hydrophobic materials (for example as an emulsion in an acceptable oil) or ion exchange resins, or as sparingly soluble derivatives, for example, as a sparingly soluble salt.

The invention also provides that a prophylactic or therapeutic agent is packaged in a hermetically sealed container such as an ampoule or sachette indicating the quantity. In one embodiment, the prophylactic or therapeutic agent is supplied as a dry sterilized lyophilized powder or water free concentrate in a hermetically sealed container and can be reconstituted, e.g., with water or saline to the appropriate concentration for administration to a subject.

In a preferred embodiment of the invention, the formulation and administration of various chemotherapeutic, biological/immunotherapeutic and hormonal therapeutic agents are known in the art and often described in the Physician's Desk Reference, 58^{th} ed. (2004).

In other embodiments of the invention, radiation therapy agents such as radioactive isotopes can be given orally as liquids in capsules or as a drink. Radioactive isotopes can also be formulated for intravenous injections. The skilled oncologist can determine the preferred formulation and route of administration.

In certain embodiments the EphA2/EphrinA1 modulators of the invention are formulated at 1 mg/ml, 5 mg/ml, 10 mg/ml, and 25 mg/ml for intravenous injections and at 5 mg/ml, 10 mg/ml, and 80 mg/ml for repeated subcutaneous administration and intramuscular injection.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

4.8.1. Gene Therapy

In specific embodiments, EphA2/EphrinA1 Modulators of the invention that are nucleotides are administered to treat, manage, or prevent a non-neoplastic hyperproliferative epithelial and/or endothelial cell disorder, including but not limited to cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis, by way of gene therapy. Gene therapy refers to therapy performed by the administration to a subject of an expressed or expressible nucleic acid. In a specific embodiment of the invention, the antisense nucleic acids are produced and mediate a prophylactic or therapeutic effect. In another specific embodiment of the invention, gene therapy is not an EphA2/EphrinA1 Modulator vaccine-based therapy (e.g., is not an EphA2- or EphrinA1 vaccine).

Any of the methods for gene therapy available in the art can be used according to the present invention. Exemplary methods are described below.

For general reviews of the methods of gene therapy, see Goldspiel et al., 1993, Clinical Pharmacy 12:488; Wu and Wu, 1991, Biotherapy 3:87; Tolstoshev, 1993, Ann. Rev. Pharmacol. Toxicol. 32:573; Mulligan, 1993, Science 260:926-932; and Morgan and Anderson, 1993, Ann. Rev. Biochem. 62:191; May, 1993, TIBTECH 11:155. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, a composition of the invention comprises EphA2 and/or EphrinA1 nucleic acids that decrease EphA2 and/or EphrinA1 expression, said nucleic acids being part of an expression vector that expresses the nucleic acid in a suitable host. In particular, such nucleic acids have promoters, preferably heterologous promoters, said promoter being inducible or constitutive, and, optionally, tissue-specific. In another particular embodiment, nucleic acid molecules are used in which the nucleic acid that decrease EphA2 and/or EphrinA1 expression and any other desired sequences are flanked by regions that promote homologous recombination at a desired site in the genome, thus providing for intrachromosomal expression of the nucleic acids that decrease EphrinA1 expression (Koller and Smithies, 1989, PNAS 86:8932; Zijlstra et al., 1989, Nature 342:435).

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy. In a specific embodiment, the nucleic acid sequences are directly administered in vivo. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., International Patent Publication Nos. WO 92/06180; WO 92/22635; WO92/203 16; WO93/14188, WO 93/20221). Alternatively, the nucleic acid can be introduced intracellularly and incorporated within host cell DNA for expression, by homologous recombination (Koller and Smithies, 1989, PNAS 86:8932; and Zijlstra et al., 1989, Nature 342:435).

In a specific embodiment, viral vectors that contain the nucleic acid sequences that decrease EphA2 and/or EphrinA1 expression are used. For example, a retroviral vector can be used (see Miller et al., 1993, Meth. Enzymol. 217:581). These retroviral vectors contain the components necessary for the correct packaging of the viral genome and integration into the host cell DNA. The nucleic acid sequences to be used in gene therapy are cloned into one or more vectors, which facilitates delivery of the nucleic acid into a subject. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Adenoviruses have the advantage of being capable of infecting non-dividing cells. Kozarsky and Wilson, 1993, Current Opinion in Genetics Development 3:499 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431; Rosenfeld et al., 1992, Cell 68:143; Mastrangeli et al., 1993, J. Clin. Invest. 91:225; International Patent Publication No. WO94/12649; and Wang et al., 1995, Gene Therapy 2:775. In a preferred embodiment, adenovirus vectors are used.

Adeno-associated virus (AAV) has also been proposed for use in gene therapy (Walsh et al., 1993, Proc. Soc. Exp. Biol. Med. 204:289-300; and U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell mediated gene transfer, spheroplast fusion, etc. Numerous techniques are known in the art for the introduction of foreign genes into cells (see, e.g., Loeffler and Behr, 1993, Meth. Enzymol. 217:599; Cohen et al., 1993, Meth. Enzymol. 217:618) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. The amount of cells envisioned for use depends on the desired effect, patient state, etc., and can be determined by one skilled in the art.

4.9 Kits

The invention provides a pharmaceutical pack or kit comprising one or more containers filled with an EphA2/EphrinA1 Modulator of the invention. Additionally, one or more other prophylactic or therapeutic agents useful for the treatment, management or prevention of a non-neoplastic hyperproliferative epithelial and/or endothelial cell disorder, including but not limited to cirrhosis, fibrosis (e.g., fibrosis of the liver, kidney, lungs, heart, retina and other viscera), asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis, or other relevant agents can also be included in the pharmaceutical pack or kit. In certain embodiments, the other prophylactic or therapeutic agent is an immunomodulatory agent (e.g., anti-IL-9 antibody). The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

5. Equivalents

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

What is claimed is:

1. A method of treating a non-neoplastic hyperproliferative epithelial or endothelial cell disorder or a symptom thereof in a patient in need thereof, said method comprising administering to said patient a therapeutically effective amount of an EphA2/EphrinA1 Modulator.

2. The method of claim 1, wherein said non-neoplastic hyperproliferative epithelial and/or endothelial cell disorder is fibrosis.

3. The method of claim 2, wherein said fibrosis is fibrosis of the liver, kidney, lungs, heart or retina.

4. The method of claim 1, wherein said non-neoplastic hyperproliferative epithelial or endothelial cell is order is cirrhosis, asthma, ischemia, atherosclerosis, diabetic retinopathy, retinopathy of prematurity, vascular restenosis, macular degeneration, rheumatoid arthritis, osteoarthritis, infantile hemangioma, verruca vulgaris, Kaposi's sarcoma, neurofibromatosis, recessive dystrophic epidermolysis bullosa, ankylosing spondylitis, systemic lupus, Reiter's syndrome, Sjogren's syndrome, endometriosis, preeclampsia, atherosclerosis, coronary artery disease, psoriatic arthropathy and psoriasis.

5. The method of claim 1, wherein said administration prevents or slows the deposition of ECM components in an epithelial cell or endothelial cell layer relative to the level of deposition of ECM components in an untreated epithelial cell or endothelial cell layer.

6. The method of claim 5, wherein said ECM component is collagen, proteoglycan, tenascin or fibronectin.

7. The method of claim 1, wherein said administration decreases EphA2-endogenous ligand binding relative to the amount of untreated EphA2-endogenous ligand binding.

8. The method of claim 7, wherein said endogenous ligand is EphrinA1.

9. The method of claim 1, wherein said administration decreases EphA2 cytoplasmic tail phosphorylation relative to the untreated level of EphA2 cytoplasmic tail phosphorylation.

10. The method of claim 1, wherein said administration increases EphA2 gene expression.

11. The method of claim 1, wherein said administration decreases EphrinA1 gene expression.

12. The method of claim 1, wherein said EphA2/EphrinA1 Modulator is an EphA2 polypeptide fragment comprising a ligand binding domain of EphA2.

13. The method of claim 1, wherein said EphA2/EphrinA1 Modulator is an EphA2 antibody or antigen binding fragment thereof.

14. The method of claim 1, wherein said EphA2/EphrinA1 Modulator is an EphrinA1 antibody or antigen binding fragment thereof.

15. The method of claim 13 or 14, wherein the said antibody is a monoclonal antibody.

16. The method of claim 15, wherein said monoclonal antibody is a human antibody.

17. The method of claim 15, wherein said monoclonal antibody is humanized.

18. The method of claim 1, wherein said EphA2/EphrinA1 Modulator is selected from the group consisting of a small molecule antagonist, enzymatic activity antagonist, EphrinA1 siRNA or eiRNA molecule, EphrinA1 antisense molecule, dominant negative EphA2 molecule, dominant negative EphrinA1 molecule, an EphA2-based vaccine and an EphrinA1-based vaccine.

19. The method of claim 1, wherein said EphA2/EphrinA1 Modulator increases EphA2 protein stability or protein accumulation.

20. The method of claim 1, further comprising the administration of one or more additional therapies for non-neoplastic hyperproliferative epithelial or endothelial cell disorders that do not alter EphA2 or EphrinA1 expression or activity.

21. The method of claim 20, wherein said additional therapies comprise an immunomodulatory agent.

22. The method of claim 21, wherein said immunomodulatory agent is an antibody that immunospecifically binds IL-9.

23. The method of claim 20, wherein said additional therapies comprise an anti-angiogenic agent.

24. The method of claim 20, wherein said additional therapies comprise an anti-inflammatory agent.

25. The method of claim 1, wherein said symptom is increased angiogenesis.


SEQUENCE LISTING (43 sequences)

SEQ ID NO: 1 - protein, 15 aa, Homo sapiens
GGGGSGGGGSGGGGS

SEQ ID NO: 2 - protein, 15 aa, Homo sapiens
ESGRSGGGGSGGGGS

SEQ ID NO: 3 - protein, 14 aa, Homo sapiens
EGKSSGSGSESKST

SEQ ID NO: 4 - protein, 15 aa, Homo sapiens
EGKSSGSGSESKSTQ

SEQ ID NO: 5 - protein, 14 aa, Homo sapiens
EGKSSGSGSESKVD

SEQ ID NO: 6 - protein, 14 aa, Homo sapiens
GSTSGSGKSSEGKG

SEQ ID NO: 7 - protein, 18 aa, Homo sapiens
KESGSVSSEQLAQFRSLD

SEQ ID NO: 8 - protein, 16 aa, Homo sapiens
ESGSVSSEELAFRSLD

SEQ ID NO: 9 - protein, 4 aa, Homo sapiens
KDEL

SEQ ID NO: 10 - protein, 4 aa, Homo sapiens
DDEL

SEQ ID NO: 11 - protein, 4 aa, Homo sapiens
DEEL

SEQ ID NO: 12 - protein, 4 aa, Homo sapiens
QEDL

SEQ ID NO: 13 - protein, 4 aa, Homo sapiens
RDEL

SEQ ID NO: 14 - protein, 7 aa, Homo sapiens
PKKKRKV

SEQ ID NO: 15 - protein, 7 aa, Homo sapiens
PQKKIKS

SEQ ID NO: 16 - protein, 5 aa, Homo sapiens
QPKKP

SEQ ID NO: 17 - protein, 4 aa, Homo sapiens
RKKR

SEQ ID NO: 18 - protein, 5 aa, Homo sapiens
KKKRK

SEQ ID NO: 19 - protein, 12 aa, Homo sapiens
RKKRRQRRRAHQ

SEQ ID NO: 20 - protein, 16 aa, Homo sapiens
RQARRNRRRRWRERQR

SEQ ID NO: 21 - protein, 19 aa, Homo sapiens
MPLTRRRPAASQALAPPTP

SEQ ID NO: 22 - protein, 15 aa, Homo sapiens
MDDQRDLISNNEQLP

SEQ ID NO: 23 - protein, 32 aa, Homo sapiens
  misc_feature (7)..(8): Xaa can be any naturally occurring amino acid
MLFNLRXXLNNAAFRHGHNFMVRNFRCGQPLX

SEQ ID NO: 24 - protein, 3 aa, Homo sapiens
AKL

SEQ ID NO: 25 - protein, 6 aa, Homo sapiens
SDYQRL

SEQ ID NO: 26 - protein, 8 aa, Homo sapiens
GCVCSSNP

SEQ ID NO: 27 - protein, 8 aa, Homo sapiens
GQTVTTPL

SEQ ID NO: 28 - protein, 8 aa, Homo sapiens
GQELSQHE

SEQ ID NO: 29 - protein, 8 aa, Homo sapiens
GNSPSYNP

SEQ ID NO: 30 - protein, 8 aa, Homo sapiens
GVSGSKGQ

SEQ ID NO: 31 - protein, 8 aa, Homo sapiens
GQTITTPL

SEQ ID NO: 32 - protein, 8 aa, Homo sapiens
GQTLTTPL

SEQ ID NO: 33 - protein, 8 aa, Homo sapiens
GQIFSRSA

SEQ ID NO: 34 - protein, 8 aa, Homo sapiens
GQIHGLSP

SEQ ID NO: 35 - protein, 8 aa, Homo sapiens
GARASVLS

SEQ ID NO: 36 - protein, 8 aa, Homo sapiens
GCTLSAEE

SEQ ID NO: 37 - protein, 16 aa, Homo sapiens
AAVALLPAVLLALLAP

SEQ ID NO: 38 - protein, 12 aa, Homo sapiens
AAVLLPVLLAAP

SEQ ID NO: 39 - protein, 15 aa, Homo sapiens
VTVLALGALAGVGVG

SEQ ID NO: 40 - DNA, 30 bp, Homo sapiens
ccagcagtaccacttccttgccctgcgccg

SEQ ID NO: 41 - DNA, 30 bp, Homo sapiens
gccgcgtcccgttccttcaccatgacgacc

SEQ ID NO: 42 - DNA, 31 bp, Homo sapiens
ccagcagtaccgcttccttgccctgcggccg

SEQ ID NO: 43 - DNA, 30 bp, Homo sapiens; identical to SEQ ID NO: 41